SEQUENCE LISTING (47 sequences)

SEQ ID NO: 1 - DNA, 441 bp, artificial sequence
  DNA sequence of VsLegH
atgtctgttgctttttctgataagcaagaagcattggttaatggttcatgggaagcctttaaaactaacatcccaaagtactctgttgttttctatactacaattttagaaaaagctccagctgctaaaaatttgttttcatttttggcaaatggtgttgatccatcaaatccaaaattgttagctcatgcagaaaaattgttcggtttagttagagattctgctgcacaattgagagcttcaggtacagttgttgcagatgctgcattaggttctgttcattcacaaaaagctgttactgatgcacaattcttggttgttaaagaagcattggttaagacattgaaagaagcagttggtgacaaatggtcagatgaattaactacagctgttgaattggcttatgatgaattagctgttgctattaaaaaggcttattaa

SEQ ID NO: 2 - protein, 146 aa, artificial sequence
  protein sequence of VsLegH
MSVAFSDKQEALVNGSWEAFKTNIPKYSVVFYTTILEKAPAAKNLFSFLANGVDPSNPKLLAHAEKLFGLVRDSAAQLRASGTVVADAALGSVHSQKAVTDAQFLVVKEALVKTLKEAVGDKWSDELTTAVELAYDELAVAIKKAY

SEQ ID NO: 3 - DNA, 468 bp, artificial sequence
  DNA sequence of LlLegH
atgtctggtgctttgactgaatctcaagctgcattggttaagtcctcatgggaagagtttaatgctaacatcccaaagcatacacatagatttttcattttggttttagaaattgcaccagctgcaaaagatttgttttcatttttgaagggtacttcagaagttccacaaaataatccagaattgcaagctcatgctggtaaagtttttaaattggtttacgaagctgcaattcaattagaagttacaggtgttgttgttactgatgctacattgaaaaatttgggttctgttcatgtttcaaaaggtgttgctgatgcacattttccagttgttaaagaagctatcttgaagactattaaagaagttgttggtgcaaaatggtctgaagaattaaattctgcttggacaatcgcatacgatgaattagcaatcgttattaagaaagaaatggatgatgctgcataa

SEQ ID NO: 4 - protein, 155 aa, artificial sequence
  protein sequence of LlLegH
MSGALTESQAALVKSSWEEFNANIPKHTHRFFILVLEIAPAAKDLFSFLKGTSEVPQNNPELQAHAGKVFKLVYEAAIQLEVTGVVVTDATLKNLGSVHVSKGVADAHFPVVKEAILKTIKEVVGAKWSEELNSAWTIAYDELAIVIKKEMDDAA

SEQ ID NO: 5 - DNA, 468 bp, Bos taurus
atgtctggtttgtctgatggtgaatggcaattggttttaaatgcttggggtaaagttgaagctgatgttgcaggtcatggtcaagaagttttgatcagattgtttactggtcatccagaaacattggaaaagttcgataagtttaaacatttgaagactgaagctgaaatgaaagcatcagaagatttgaagaaacatggtaacactgttttgacagctttgggtggtattttgaaaaagaaaggtcatcatgaagcagaagttaaacatttggctgaatctcatgcaaataagcataagatccctgttaagtatttggagtttatttcagatgctatcatccatgtcttacatgcaaaacatccatctgattttggtgctgatgcacaagctgcaatgtctaaagcattggaattgtttagaaacgatatggctgcacaatacaaagttttgggttttcatggttaa

SEQ ID NO: 6 - DNA, 765 bp, artificial sequence
  DNA sequence of HLTev-VsLegH
atgcaccaccaccaccaccacagcggcgcttttgaatttaagctgccggacattggcgaaggcatccacgaaggtgaaattgtcaaatggtttgtgaaaccgggcgatgaagtgaacgaagacgatgtattgtgcgaagtgcaaaatgacaaggcggttgtcgaaattccctccccggtcaaagggaaagtgcttgaaatcctcgtcccggagggaacagtggcaacggtcgggcaaacgctcatcacgctcgatgcgccgggttatgaaaacatgacgaccggcagcgacaccggcgaaaacctgtacttccagggtggatccatgtctgttgctttttctgataagcaagaagcattggttaatggttcatgggaagcctttaaaactaacatcccaaagtactctgttgttttctatactacaattttagaaaaagctccagctgctaaaaatttgttttcatttttggcaaatggtgttgatccatcaaatccaaaattgttagctcatgcagaaaaattgttcggtttagttagagattctgctgcacaattgagagcttcaggtacagttgttgcagatgctgcattaggttctgttcattcacaaaaagctgttactgatgcacaattcttggttgttaaagaagcattggttaagacattgaaagaagcagttggtgacaaatggtcagatgaattaactacagctgttgaattggcttatgatgaattagctgttgctattaaaaaggcttattaa

SEQ ID NO: 7 - protein, 254 aa, artificial sequence
  protein sequence of HLTev-VsLegH
MHHHHHHSGAFEFKLPDIGEGIHEGEIVKWFVKPGDEVNEDDVLCEVQNDKAVVEIPSPVKGKVLEILVPEGTVATVGQTLITLDAPGYENMTTGSDTGENLYFQGGSMSVAFSDKQEALVNGSWEAFKTNIPKYSVVFYTTILEKAPAAKNLFSFLANGVDPSNPKLLAHAEKLFGLVRDSAAQLRASGTVVADAALGSVHSQKAVTDAQFLVVKEALVKTLKEAVGDKWSDELTTAVELAYDELAVAIKKAY

SEQ ID NO: 8 - DNA, 792 bp, artificial sequence
  DNA sequence of HLTev-LlLegH
atgcaccaccaccaccaccacagcggcgcttttgaatttaagctgccggacattggcgaaggcatccacgaaggtgaaattgtcaaatggtttgtgaaaccgggcgatgaagtgaacgaagacgatgtattgtgcgaagtgcaaaatgacaaggcggttgtcgaaattccctccccggtcaaagggaaagtgcttgaaatcctcgtcccggagggaacagtggcaacggtcgggcaaacgctcatcacgctcgatgcgccgggttatgaaaacatgacgaccggcagcgacaccggcgaaaacctgtacttccagggtggatccatgtctggtgctttgactgaatctcaagctgcattggttaagtcctcatgggaagagtttaatgctaacatcccaaagcatacacatagatttttcattttggttttagaaattgcaccagctgcaaaagatttgttttcatttttgaagggtacttcagaagttccacaaaataatccagaattgcaagctcatgctggtaaagtttttaaattggtttacgaagctgcaattcaattagaagttacaggtgttgttgttactgatgctacattgaaaaatttgggttctgttcatgtttcaaaaggtgttgctgatgcacattttccagttgttaaagaagctatcttgaagactattaaagaagttgttggtgcaaaatggtctgaagaattaaattctgcttggacaatcgcatacgatgaattagcaatcgttattaagaaagaaatggatgatgctgcataa

SEQ ID NO: 9 - protein, 263 aa, artificial sequence
  protein sequence of HLTev-LlLegH
MHHHHHHSGAFEFKLPDIGEGIHEGEIVKWFVKPGDEVNEDDVLCEVQNDKAVVEIPSPVKGKVLEILVPEGTVATVGQTLITLDAPGYENMTTGSDTGENLYFQGGSMSGALTESQAALVKSSWEEFNANIPKHTHRFFILVLEIAPAAKDLFSFLKGTSEVPQNNPELQAHAGKVFKLVYEAAIQLEVTGVVVTDATLKNLGSVHVSKGVADAHFPVVKEAILKTIKEVVGAKWSEELNSAWTIAYDELAIVIKKEMDDAA

SEQ ID NO: 10 - DNA, 324 bp, artificial sequence
  DNA sequence of HLTev
atgcaccaccaccaccaccacagcggcgcttttgaatttaagctgccggacattggcgaaggcatccacgaaggtgaaattgtcaaatggtttgtgaaaccgggcgatgaagtgaacgaagacgatgtattgtgcgaagtgcaaaatgacaaggcggttgtcgaaattccctccccggtcaaagggaaagtgcttgaaatcctcgtcccggagggaacagtggcaacggtcgggcaaacgctcatcacgctcgatgcgccgggttatgaaaacatgacgaccggcagcgacaccggcgaaaacctgtacttccagggtggatcc

SEQ ID NO: 11 - protein, 15 aa, artificial sequence
  protein sequence of FBA-tag
GVEQILKRKTGVIVG

SEQ ID NO: 12 - protein, 4 aa, artificial sequence
  protein sequence of SKIK-tag
SKIK

SEQ ID NO: 13 - DNA, 43 bp, artificial sequence
  primer SKIK-VsLegH-F
atgtctaaaataaaatctgttgctttttctgataagcaagaag

SEQ ID NO: 14 - DNA, 76 bp, artificial sequence
  primer FBA-VsLegH-F
atgggtgttgaacaaatcttaaagagaaagaccggtgtcatcgttggttctgttgctttttctgataagcaagaag

SEQ ID NO: 15 - DNA, 37 bp, artificial sequence
  primer SKIK-LlLegH-F
atgtctaaaataaaatctggtgctttgactgaatctc

SEQ ID NO: 16 - DNA, 70 bp, artificial sequence
  primer FBA-LlLegH F
atgggtgttgaacaaatcttaaagagaaagaccggtgtcatcgttggttctggtgctttgactgaatctc

SEQ ID NO: 17 - DNA, 38 bp, artificial sequence
  primer SKIK-BtMyg-F
atgtctaaaataaaatctggtttgtctgatggtgaatg

SEQ ID NO: 18 - DNA, 71 bp, artificial sequence
  primer FBA-BtMyg-F
atgggtgttgaacaaatcttaaagagaaagaccggtgtcatcgttggttctggtttgtctgatggtgaatg

SEQ ID NO: 19 - DNA, 33 bp, artificial sequence
  primer pYES2-R
ttttttaagcttaatattccctatagtgagtcg

SEQ ID NO: 20 - protein, 155 aa, artificial sequence
  protein sequence of BtMyg
MSGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 21 - protein, 161 aa, artificial sequence
  protein sequence of FBA-VsLegH
MGVEQILKRKTGVIVGSVAFSDKQEALVNGSWEAFKTNIPKYSVVFYTTILEKAPAAKNLFSFLANGVDPSNPKLLAHAEKLFGLVRDSAAQLRASGTVVADAALGSVHSQKAVTDAQFLVVKEALVKTLKEAVGDKWSDELTTAVELAYDELAVAIKKAY

SEQ ID NO: 22 - protein, 150 aa, artificial sequence
  protein sequence of SKIK-VsLegH
MSKIKSVAFSDKQEALVNGSWEAFKTNIPKYSVVFYTTILEKAPAAKNLFSFLANGVDPSNPKLLAHAEKLFGLVRDSAAQLRASGTVVADAALGSVHSQKAVTDAQFLVVKEALVKTLKEAVGDKWSDELTTAVELAYDELAVAIKKAY

SEQ ID NO: 23 - protein, 154 aa, artificial sequence
  protein sequence of LaLegH1
MAILTDVQVALVKSSFEEFNANIPKHTHRFFTLVLEIAPTAKDLFSFLKGTNEVPQNNPDLQAHAGKVFKLTYEAAIQLQVNGAVATDATLKNLGSVHVSKGVIDAHFPVVKEAILKTIKEVVGDKWSEELNNAWTIAYDELAIIIKKEMKDAA

SEQ ID NO: 24 - protein, 149 aa, artificial sequence
  protein sequence of LaLegH2
MGALTESQAALVKSSFEEFNANIPKHTQRFFILVLEIAPAAKDLFSFLKGTSEVPQNNPELQAHAGKVFKLVYEAAIQLQVTGAVVADATLKNLGSIHVSKGVVDAHFPVVKEAILKTIKEVVGDKWSEELGNAWTIAYDELAIVIKRG

SEQ ID NO: 25 - protein, 154 aa, artificial sequence
  protein sequence of LlLegH1
MGVLTDVQVALVKSSFEEFNANIPKNTHRFFTLVLEIAPGAKDLFSFLKGSSEVPQNNPDLQAHAGKVFKLTYEAAIQLQVNGAVASDATLKSLGSVHVSKGVVDAHFPVVKEAILKTIKEVVGDKWSEELNTAWTIAYDELAIIIKKEMKDAA

SEQ ID NO: 26 - protein, 148 aa, artificial sequence
  protein sequence of LalLegH
MGALTETQAALVKSSWEEFNANIPKHTHLFFTLVLEIAPAAKDLFSFLKGTSEVPLNSPELQAHAGKVFKLVYEAAIQLQVNGVIVTDATSKNLGSLHVSKGVVDAHFPVVKEAILKTIKEVVGAKWSEELSTAWTIAYDELAIVIKN

SEQ ID NO: 27 - DNA, 462 bp, artificial sequence
  nucleic acid sequence of LaLegH1
atggcgattttgacggatgttcaggttgccctcgttaagtcctcgtttgaggagtttaacgccaacattccgaaacacacacatcgcttctttacgctggtcttggaaattgctcctactgccaaagacctgtttagcttcctgaaaggcacgaatgaagtgccgcaaaacaacccggatttacaggcacatgctgggaaagtgttcaaactgacctatgaagcagcgattcagcttcaagtgaatggagctgttgcgactgatgcaaccctgaagaatctgggttcagtccatgtctctaaaggtgtgatcgatgcacactttccagtggtgaaagaagcgattctgaaaaccatcaaagaagtagtaggcgacaaatggagtgaagaactgaacaatgcgtggaccattgcctacgacgaattagcgatcatcatcaagaaggagatgaaagatgcggcc

SEQ ID NO: 28 - DNA, 462 bp, artificial sequence
  nucleic acid sequence of LlLegH1
atgggcgttctgaccgatgtccaagttgctctggtgaaatcgagctttgaggagtttaacgcgaacattccgaagaatacccatcgcttcttcacgttagtgctggaaattgcaccaggtgctaaagatctgttttccttcctcaaagggagttctgaagtaccgcagaataacccggacttacaagcgcatgcaggcaaagtctttaagttgacgtatgaagccgctattcagcttcaggtgaatggtgcagtagcctcagatgcgacactgaaaagcctgggtagtgtgcacgtgtcgaaaggcgtcgttgatgcgcactttcctgtggttaaagaagcgattctgaaaaccatcaaggaagtcgtaggagacaaatggagcgaagaactgaacactgcatggaccattgcctacgacgaattggccatcatcatcaagaaagagatgaaagatgcggcc

SEQ ID NO: 29 - DNA, 765 bp, artificial sequence; identical to SEQ ID NO: 6
  nucleic acid sequence of HLTev-VsLegH

SEQ ID NO: 30 - DNA, 792 bp, artificial sequence; identical to SEQ ID NO: 8
  nucleic acid sequence of HLTev-LlLegH

SEQ ID NO: 31 - protein, 144 aa, artificial sequence
  protein sequence of LegH C1
MGAFTEKQEALVSSSFEAFKANIPQYSVVFYNSILEKAPAAKDLFSFLANGVDPTNPKLTGHAEKLFALVRDSAGQLKTNGTVVADAALVSIHAQKAVTDPQFVVVKEALLKTIKEAVGGNWSDELSSAWEVAYDELAAAIKKA

SEQ ID NO: 32 - protein, 145 aa, artificial sequence
  protein sequence of LegH C2
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 33 - protein, 145 aa, artificial sequence
  protein sequence of LegH C3
MGAFTDKQEALVSSSFEAFKTNIPQYSVVFYTSILEKAPVAKDLFSFLANGVDPTNPKLTGHAEKLFGLVRDSAGQLKASGTVVIDAALGSIHGQKAITDPQFVVVKEALLKTIKEAVGDKWSDELSSAWEVAYDELAAAIKKAF

SEQ ID NO: 34 - protein, 144 aa, artificial sequence
  protein sequence of LegH A
MVAFTEKQDALVSSSFEAFKANIPQYSVVFYTSILEKAPAAKDLFSFLANGVDPTNPKLTGHAEKLFALVRDSAGQLKASGTVVADAALGSVHAQKAVTDPQFVVVKEALLKTIKAAVGDKWSDELSRAWEVAYDELAAAIKKA

SEQ ID NO: 35 - protein, 115 aa, artificial sequence
  protein sequence of VsLegH9
MNRILEKAPAAKNLFSFLANGVDPSNPKLLAHAEKLFGLVRDSAAQLRASGTVVADAALGSVHSQKAVTDAQFLVVKEALVKTLKEAVGDKWSDELATAVELAYDELAVAIKKAY

SEQ ID NO: 36 - protein, 267 aa, artificial sequence
  protein sequence of VsLegH10
MVHGKHSRQTFLNTVLSSTPREILEKAPAAKNLFSFLANGVDPSNPKLLAHAEKLFGLVRDSAAQLRASGTVVADAALGSVHSQKAVTDAQFLVVKEALVKTLKEAVGDKWSDELATAVELAYDELAKQEQLVNSSWEAFKGNLSHNSATFFNLVLEKEPGAKNLFSFLGDGVDPNNPKIAAHAEKLFGTVRDAAVQLKTKGEVVAEAKLGSVHAEKKVTDPQFAVIKEAVLKTIKEAVGEKWNEEVNDAWAQAYDAMAAAIKKTIA

SEQ ID NO: 37 - protein, 83 aa, artificial sequence
  protein sequence of VsLegH61
MNRILEKAPIAKNLFSFLANGVDPSNPKLVAHAEKLFGLVRDSAAQLRASGTVVADAALGSVHSQKAVTDAQYLIPLVTIVLW

SEQ ID NO: 38 - protein, 146 aa, artificial sequence
  protein sequence of bact. hemoglobin from Vitreoscilla stercoraria
MLDQQTINIIKATVPVLKEHGVTITTTFYKNLFAKHPEVRPLFDMGRQESLEQPKALAMTVLAAAQNIENLPAILPAVKKIAVKHCQAGVAAAHYPIVGQELLGAIKEVLGDAATDDILDAWGKAYGVIADVFIQVEADLYAQAVE

SEQ ID NO: 39 - DNA, 432 bp, artificial sequence
  nucleic acid sequence of LegH C1
atgggcgcctttaccgagaaacaagaagcgctggtgtcaagcagcttcgaagcgtttaaagccaacattccgcagtattccgtggtgttctacaactcgattctggaaaaggcccctgctgccaaagacctgttcagctttctggccaatggcgtagatccaaccaacccgaaactcaccggtcatgcggaaaagctgtttgcgttggttcgcgatagtgctggtcagctgaaaacgaatggcacagttgtggctgatgcagccttagtctccattcacgcacagaaagcggtcactgatccgcaatttgtcgttgtgaaagaagcgttgctgaaaacgatcaaagaggcagtaggagggaattggagtgacgaactttcgtctgcatgggaagttgcgtatgacgagttagctgcagcgatcaagaaagcg

SEQ ID NO: 40 - DNA, 435 bp, artificial sequence
  nucleic acid sequence of LegH C2
atgggagcgtttacggagaaacaggaagcactggttagttccagctttgaggccttcaaagccaacattccgcaatattccgtggtgttctacaccagcattctggagaaagcgcctgcagccaaagacctgttttcgttcctctctaatggcgttgatccgtcaaacccgaaattaaccggtcatgcggaaaagctgtttgggttggtacgcgatagtgcgggtcaactgaaggcgaatggcacagtcgtcgctgatgcagccttaggtagcattcacgcgcagaaagccatcactgatccacagtttgtggtggtcaaagaagcgctgttgaaaaccatcaaagaagctgttggcgacaaatggagcgatgaactgtcgtctgcatgggaagtagcctatgacgaacttgcagctgctatcaagaaagcgttc

SEQ ID NO: 41 - DNA, 435 bp, artificial sequence
  nucleic acid sequence of LegH C3
atgggtgccttcacagacaaacaggaagccttggtttcctctagctttgaggctttcaagaccaacattccgcagtattcggtcgtgttctacacctccatcctggagaaagctccagtcgccaaagacctgttttcgttcttagcaaacggcgttgatccgacgaatccgaaactgactggacatgcggagaaactgtttggcttggttcgcgattctgccggtcaactcaaagcgagtggtaccgtggtcattgatgcggcgttaggcagcattcacgggcaaaaggccattaccgatcctcagtttgtggtggtgaaagaagcactgcttaaaacgatcaaagaagctgtaggcgacaaatggagtgatgaactgagctcagcgtgggaagtagcgtatgatgaactggcagcagccatcaagaaagcgttt

SEQ ID NO: 42 - DNA, 432 bp, artificial sequence
  nucleic acid sequence of LegH A
atggttgcgtttacggagaaacaggatgctctcgttagctcgagcttcgaagcctttaaagcgaacattccgcagtattcggtggtgttctacacctccattctggagaaagctccagctgcgaaagacttgttctcctttctggccaatggggtcgatcctaccaacccgaaattgacaggacacgcagaaaagctgtttgcgttagtacgcgatagtgccggtcaactgaaagcctcaggtaccgttgtcgccgatgctgcattaggcagtgttcatgcgcagaaagcggtgactgatccgcaatttgtggtggtgaaagaagccctgcttaagacgatcaaagcagcggtaggcgacaaatggagcgatgaactgtctcgtgcatgggaagtcgcgtatgacgaactggcagcagcgatcaagaaagcc

SEQ ID NO: 43 - DNA, 348 bp, artificial sequence
  nucleic acid sequence of VsLegH9
atgaaccgcattctggagaaagccccagcagcgaagaaccttttcagctttctggccaatggtgtcgatccgtcgaatccgaaactcctggcacatgcagagaagctgttcgggttagtgcgcgatagtgctgcccaactgcgtgcttcaggcaccgttgttgccgatgcagcgttaggctctgtacactcccagaaagcggttaccgatgcgcagtttctcgtcgtcaaagaagcgctggtgaaaacgctgaaagaagctgtgggtgacaaatggagcgacgaattggcaactgcggtggaactggcgtatgacgaattggccgtagcgatcaagaaagcctactaa

SEQ ID NO: 44 - DNA, 801 bp, artificial sequence
  nucleic acid sequence of VsLegH10
atggtacacggcaaacacagccgtcaaacgttcctgaataccgtgcttagctcaactcctcgcgagattctcgaaaaagccccagcagctaaaaacctgtttagctttctggcgaatggcgttgatccgagtaatccgaaactgttagcacatgctgaaaagctgtttgggctggttcgcgactcagcggcgcaattacgcgcatctggcacggtagtggctgatgccgcattggggagtgtccattcccagaaagccgtgaccgatgcgcagtttctggttgtgaaggaagcactggtgaaaacgctgaaagaagccgttggtgacaaatggtcggatgaactggcaacagcggttgaactcgcctatgacgagttagcgaaacaggaacagctggtgaatagctcttgggaagcgttcaaaggcaacttgtcgcacaatagcgcgaccttcttcaaccttgtcctggagaaagaaccaggtgcgaaaaacctgttctcctttctgggagatggagtcgatcccaacaacccgaaaattgcggcacatgccgagaaactctttggcactgtacgtgatgccgctgttcagttaaagaccaaaggtgaggtagtggcggaagccaaacttggttcggtgcatgcggagaagaaagtcacagatccgcagtttgcggtgatcaaagaagcggtgttgaaaaccatcaaggaagccgtcggtgaaaaatggaacgaagaggttaatgacgcgtgggctcaagcctacgacgcaatggcagctgctatcaaaaagaccattgcc

SEQ ID NO: 45 - DNA, 249 bp, artificial sequence
  nucleic acid sequence of VsLegH61
atgaatcgcatccttgagaaagctccgattgcgaagaacctgttctcctttctggccaatggtgtggacccttcgaacccgaaactggtcgcacatgccgaaaagctgtttggccttgttcgcgactcagctgctcaactccgtgcttctggtactgtagtggctgatgcggcattaggctcagtacacagccagaaagccgttactgatgcgcagtacctgatcccactggtgaccatcgtcttgtgg

SEQ ID NO: 46 - DNA, 438 bp, artificial sequence
  nucleic acid of bact. hemoglobin from Vitreoscilla stercoraria
atgctggatcagcagaccattaacatcatcaaagccactgttccggtactgaaagaacacggcgttaccattacgaccaccttctacaagaatctgtttgcgaaacatccggaagttcgtcccctctttgacatgggtcgccaagaaagcttggaacagccgaaagctctggcgatgacagtgttagctgcagcccagaacatcgagaatctgccagcgattcttcctgcggtgaagaaaattgccgtgaaacactgccaagcgggtgtagctgcggcacattacccgattgttggccaggaactgttaggtgccatcaaagaggtcttgggcgatgcagctacggacgatatcctggatgcctggggaaaagcgtatggggtcattgccgatgtgttcattcaggtggaagcagacctgtatgcacaagcggtcgag

SEQ ID NO: 47 - DNA, 7010 bp, artificial sequence
  nucleic acid sequence of pTTB2-HLTev-VsLegH
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccttaaggaacgtacagacggcttaaaagcctttaaaaacgtttttaaggggtttgtagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctccaaatattcttatttagaaaagcaaatctaaaattatctgaaaagggaatgagaatattgtctgaatccagcgcaagaaccgaaatgaaaatcagcttgcccgaaaacctagtagctgaattggatggtgtagcgatgcgggagaaacgaagcagaaacgaactgatatcacaagcagtgagagcgtatgtcagcgaacgaacaactcgtcacaaccgtgatttgatgagacgcggctatatggaaatggcgaaaatcaacctgaatatttcttctgaggctcactttgcggagtgcgaggctgaaacgacagttgagcgcttagtcagcggaggataaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcattgccagtcggggatattaaaaagagtataggtttttattgggataaagtaggtttcactttggttcaccatgaagatggattcgcagttctaatgtgtaatgaggttcggattcatctatgggaggcaagtgatgaaggctggcgcctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattagcttttttcaacaaataaaaagctaaaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgcgcctcgcagagcacacactttatgaatataaagtatagtgtgttatactttacttggaagtggttgccggaaagagcgaaaatgcctcacatttgtgccacctaaaaaggagcgatttacatatgagttatgcagtttgtagaatgcaaaaagtgaaatcagctggactaaaaggcatgcaatttcataatcaaagagagcgaaaaagtagaacgaatgatgatattgaccatgagcgaacacgtgaaaattatgatttgaaaaatgataaaaatattgattacaacgaacgtgtcaaagaaattattgaatcacaaaaaacaggtacaagaaaaacgaggaaagatgctgttcttgtaaatgagttgctagtaacatctgaccgagatttttttgagcaactggatcctgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatataaagtgatagcggtacccatatgcaccaccaccaccaccacagcggcgcttttgaatttaagctgccggacattggcgaaggcatccacgaaggtgaaattgtcaaatggtttgtgaaaccgggcgatgaagtgaacgaagacgatgtattgtgcgaagtgcaaaatgacaaggcggttgtcgaaattccctccccggtcaaagggaaagtgcttgaaatcctcgtcccggagggaacagtggcaacggtcgggcaaacgctcatcacgctcgatgcgccgggttatgaaaacatgacgaccggcagcgacaccggcgaaaacctgtacttccagggtggatccatgtctgttgctttttctgataagcaagaagcattggttaatggttcatgggaagcctttaaaactaacatcccaaagtactctgttgttttctatactacaattttagaaaaagctccagctgctaaaaatttgttttcatttttggcaaatggtgttgatccatcaaatccaaaattgttagctcatgcagaaaaattgttcggtttagttagagattctgctgcacaattgagagcttcaggtacagttgttgcagatgctgcattaggttctgttcattcacaaaaagctgttactgatgcacaattcttggttgttaaagaagcattggttaagacattgaaagaagcagttggtgacaaatggtcagatgaattaactacagctgttgaattggcttatgatgaattagctgttgctattaaaaaggcttattaagaattcgagctccgtcgacctcgaggggctagccgctgcagttgaattcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2023/0416764 A1
(43) Pub. Date: Dec. 28, 2023

(54) METHOD OF PRODUCING GLOBIN POLYPEPTIDE RECOMBINANTLY AND MEAT SUBSTITUTE FOOD PRODUCT

(71) Applicant: PLANEAT FOODS PTE. LTD., Singapore (SG)

(72) Inventors: Tuck Seng WONG, South Yorkshire (GB); Kang Lan TEE, South Yorkshire (GB); Margit LANGWALLNER, Singapore (SG); Madhavan NALLANI, Singapore (SG); Christoph LANGWALLNER, Singapore (SG)

(73) Assignee: PLANEAT FOODS PTE. LTD., Singapore (SG)

(21) Appl. No.: 18/255,791

(22) PCT Filed: Dec. 2, 2021
(86) PCT No.: PCT/EP2021/083972

§ 371 Date: Jun. 2, 2023

(30) Foreign Application Priority Data
Dec. 2, 2020 (EP) 20211249.6

(51) Int. Cl. C12N 15/70; C12N 15/81; A23J 3/20; C12N 15/75; A23L 33/145; A23L 33/135; C12N 1/18; C12N 1/20

(52) U.S. Cl. CPC C12N 15/70; C12N 15/81; A23J 3/20; C12N 15/75; A23L 33/145; A23L 33/135; C12N 1/185; C12N 1/205

(57) ABSTRACT

The present invention refers to a method of producing globin polypeptide recombinantly and a method of producing globin polypeptide with a cell-free translation system. Further, the present invention provides a food product, preferably a meat substitute food product. The present invention is also directed to a vector and a system for recombinant globin polypeptide production as well as to a cell comprising a recombinant expression vector or one or more recombinant nucleic acid molecule(s).

### FIELD OF THE INVENTION

The present invention refers to a method of producing globin polypeptide recombinantly and a method of producing globin polypeptide with a cell-free translation system, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof. Further, the present invention provides a food product, preferably a meat substitute food product. The present invention is also directed to a vector and a system for recombinant globin polypeptide production as well as to a cell comprising a recombinant expression vector or one or more recombinant nucleic acid molecule(s).

### BACKGROUND OF THE INVENTION

Methods using E. coli to produce human hemoglobin have been developed, however, production of hemoglobin in this way has proved to be inefficient over a long period of time as the removal of endotoxin from E. coli products is very costly. Yeast strains have also been used to produce hemoproteins, but production on such hemoproteins have proven to be difficult due to the lack of simultaneously abundant amounts of both apoproteins and heme in such production systems.

However, Martinez et al. describes human hemoglobin production by Saccharomyces cerevisiae.

Further, US 2009/0098607A1 describes a production method of a functional recombinant human hemoglobin.

Thus, as can be seen from these references, a method or system of producing polypeptides recombinantly with a host cell or with a cell-free translation system has so far been established for human hemoglobin, but good alternatives for producing respective globins from plants are lacking, especially for leghemoglobins. The same applies for providing the use of the respective proteins in food products, especially meat-substitute food products.

However, there is an increased need for such products, as many people are choosing nowadays to limit the amount of meat in their diet. Specifically, people are looking to reduce the amount of animal fat in their diets. Animal fat is a primary source of saturated fat, which raises blood cholesterol.

Despite the desire to limit meat in the diet, people nonetheless want to eat products that were traditionally meat-based products, such as burgers. Non-meat burgers are known for example to be made from vegetables, nut, dairy products, mushrooms, grain or textured vegetable protein.

Fat in a non-meat burger, and other meat substitutes, plays a vital role in a variety of sensory attributes, including juiciness, mouth feel and flavor. When a meat substitute product has lower amounts of fat, there is a tendency for the cooked product to be less desirable in regards to juiciness, mouth feel and flavor. On the contrary, when a meat substitute product has an optimal amount of fat, it is more desirable in terms of juiciness, mouth feel and flavor.

Consequently, there is a need for methods of producing substances that can serve as ingredients in such meat-substitute products and the inventors of the present invention comply with this need by providing methods and products leading to specifically improved meat-like aroma, appearance, flavor, texture and taste.

### SUMMARY OF THE INVENTION

In a first aspect, the present invention provides a method of producing globin polypeptide recombinantly, comprising:
transforming a host cell with an expression vector comprising
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention provides a method of producing globin polypeptide recombinantly, comprising:
transforming a host cell with an expression vector comprising one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof, wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment of the method of producing globin polypeptide recombinantly, the one or more nucleic acid sequence(s) encode(s) a leghemoglobin from a Vigna plant, preferably a Vigna plant selected from the group consisting of Vigna ambacensis, Vigna angivensis, Vigna filicaulis, Vigna friesiorum, Vigna gazensis, Vigna hosei, Vigna luteola, Vigna membranacea, Vigna monantha, Vigna racemosa, Vigna subterranea, and Vigna unguiculata, more preferably from Vigna subterranea.

In one further embodiment of the method of producing globin polypeptide recombinantly, the one or more nucleic acid sequence(s) encode(s) a leghemoglobin from a lupin, preferably from a lupin selected from the group consisting of Lupinus albus, Lupinus angustifolius, Lupinus micranthus, Lupinus luteus, Lupinus hispanicus, Lupinus cosentinii, Lupinus digitatus, Lupinus princei, Lupinus pilosus, Lupinus palaestinus, Lupinus atlanticus, Lupinus mutabilis, Lupinus texensis, and Lupinus nootkatensis, more preferably Lupinus luteus.

In one embodiment of the method of producing globin polypeptide recombinantly, the one or more nucleic acid sequence(s) encode(s) a myoglobin from bovine, preferably a myoglobin from Bos taurus, Bos primigenius, Bos javanicus, Bos gaurus, Bos frontalis, Bos grunniens, Bos mutus, and Bos sauveli, more preferably from Bos taurus.

In one further embodiment of the method of producing globin polypeptide recombinantly, the one or more nucleic acid sequence(s) encode(s) a bacterial hemoglobin having at least 70% sequence identity with the bacterial hemoglobin of Vitreoscilla stercoraria, preferably the one or more nucleic acid sequence(s) encode(s) the bacterial hemoglobin of Vitreoscilla stercoraria, of Vitreoscilla sp. HG1, or of the Vitreoscilla sp strain C1.

Also, in one embodiment of the method of producing globin polypeptide recombinantly, the host cell is a bacterial host cell, preferably a bacterial host cell selected from the group consisting of Escherichia coli, Bacillus subtilis and Lactococcus lactis.

In another embodiment of the method of producing globin polypeptide recombinantly, the host cell is a yeast host cell, preferably a yeast host cell of the genus Saccharomyces, Pichia, Candida, Torulopsis or Hansenula, more preferably of Saccharomyces cerevisiae.

It is also preferred for the method of producing globin polypeptide recombinantly of the present invention, that said globin polypeptide is/are heterologous with respect to said host cell.

Additionally, in one embodiment of the method of producing globin polypeptide recombinantly, it is preferred that said one or more nucleic acid sequence(s) is/are under regulation of a promoter functional in bacteria or yeast, preferably wherein said promoter is a bacterial promoter, more preferably wherein the bacterial promoter is selected from the group consisting of the araBAD promoter, lac promoter, lacUV5 promoter, phoA promoter, pL promoter, pR promoter, rhaBAD promoter, Sp6 promoter, T3 promoter, T5 promoter, T7 promoter, T7lac promoter, tac promoter, tet promoter, trc promoter and the trp promoter, even more preferably the T7lac promoter; or, preferably wherein said promoter is a yeast promoter, more preferably wherein the yeast promoter is selected from the group consisting of the GAL1 promoter, GAL10 promoter, GALL promoter, GALS promoter, CTR1 promoter, CTR3 promoter, CUP1 promoter, CYC1 promoter, MET25 promoter, the promoter of glyceraldehyde 3-phosphate dehydrogenase (GPD), the promoter of alcohol dehydrogenase 1 (ADH1), the promoter of transcriptional elongation factor EF-1α (TEF1), the promoter of transcriptional elongation factor EF-1α (TEF2), the promoter of phosphoglycerate kinase (PGK1), the promoter of triose phosphate isomerase (TPI1), the promoter of hexose transporter (HXT7), the promoter of pyruvate kinase 1 (PYK1), and the promoter of triose phosphate dehydrogenase (TDH3), even more preferably the GAL1 promoter.

In one embodiment of the method of producing globin polypeptide recombinantly, the one or more nucleic acid sequence(s) encoding the globin polypeptide comprise(s) or consist(s) of a sequence selected from the group consisting of SEQ ID NO: 1, SEQ ID NO: 3, SEQ ID NO: 5, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46. The present invention also comprises fragments of these mentioned sequences.

In a further aspect, the present invention provides a method of producing globin polypeptide with a cell-free translation system, comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
translating the one or more nucleic acid sequence(s) with the cell-free translation system, and
recovering the globin polypeptide from the cell-free translation system.

In one embodiment, the present invention provides a method of producing globin polypeptide with a cell-free translation system, comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine and any combination thereof,
translating the one or more nucleic acid sequence(s) with the cell-free translation system, and
recovering the globin polypeptide from the cell-free translation system.

In one embodiment of this method of producing globin polypeptide with a cell-free translation system, the cell-free translation system is a bacterial cell-free system or a yeast cell-free system.

In a further aspect, the present invention provides a food product, preferably a meat substitute food product, comprising:
one or more globin protein(s), wherein said one or more globin protein(s) is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein(s) from a plant, more preferably one or more protein(s) from a legume or a grain,
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optionally one or more cell based protein.

In one embodiment, the present invention provides a food product, preferably a meat substitute food product, comprising:
one or more globin protein(s), wherein said one or more globin protein(s) is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optionally one or more cell based protein.

In one embodiment of the food product of the present invention, the one or more globin protein(s) comprise(s) or consist(s) of a sequence set forth in SEQ ID NO: 2, SEQ ID NO: 4, SEQ ID NO: 23, SEQ ID NO: 25, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 38. In one embodiment of the food product of the present invention, the one or more globin protein(s) comprise(s) or consist(s) of a sequence set forth in SEQ ID NO: 2 or SEQ ID NO: 4. The present invention also comprises fragments of these mentioned sequences.

In a further aspect, the present invention provides a vector comprising:
a nucleic acid sequence encoding a transcriptional activator,
one or more nucleic acid sequence(s) encoding globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof;
a nucleic acid sequence encoding one or more tag protein(s), preferably one or more tag protein(s) selected from the group consisting of Biotin-carboxy carrier protein (BCCP), Calmodulin, Chitin-binding domain (CBD), Glutathione-S-transferase (GST), HaloTag, Maltose-binding protein (MBP), Polyhistidine tag, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, Nutilization substance A (NusA), Peptidyl-prolyl cis-trans isomerase B (PpiB), Thioredoxin (Trx), and SUMO, more preferably the tag protein polyhistidine-tag and/or the lipoyl domain from Bacillus stearothermophilus E2p.

In one embodiment, the present invention provides a vector comprising:
a nucleic acid sequence encoding a transcriptional activator,
one or more nucleic acid sequence(s) encoding globin polypeptide, wherein said globin polypeptide is/are selected from the group consisting of a leghemoglobin from a Vigna plant, a leghemoglobin from a lupin, a myoglobin from bovine and any combination thereof;
a nucleic acid sequence encoding one or more tag protein(s), preferably one or more tag protein(s) selected from the group consisting of Biotin-carboxy carrier protein (BCCP), Calmodulin, Chitin-binding domain (CBD), Glutathione-S-transferase (GST), HaloTag, Maltose-binding protein (MBP), Polyhistidine tag, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, N-utilization substance A (NusA), Peptidyl-prolyl cis-trans isomerase B (PpiB), Thioredoxin (Trx), and SUMO, more preferably the tag protein Polyhistidine-tag and/or the lipoyl domain from Bacillus stearothermophilus E2p.

In a further aspect, the present invention provides a system for recombinant globin polypeptide production, comprising:
a bacterial or yeast cell or a cell-free translation system; and
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and, a bacterial hemoglobin having at least 70% sequence identity with the bacterial hemoglobin of Vitreoscilla stercoraria, and any combination thereof.

In one embodiment, the present invention provides a system for recombinant globin polypeptide production, comprising:
a bacterial or yeast cell or a cell-free translation system; and
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof.

In another aspect, the present invention provides a cell comprising a recombinant expression vector or one or more recombinant nucleic acid molecule(s), comprising:
a nucleic acid sequence encoding a transcriptional activator,
one or more nucleic acid sequence(s) encoding a globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
a nucleic acid sequence encoding at least one polypeptide involved in the biosynthesis of said globin polypeptide.

In one embodiment, the present invention provides a cell comprising a recombinant expression vector or one or more recombinant nucleic acid molecule(s), comprising:
a nucleic acid sequence encoding a transcriptional activator,
one or more nucleic acid sequence(s) encoding a globin polypeptide, wherein said globin polypeptide is/are selected from the group consisting of a leghemoglobin from a Vigna plant, a leghemoglobin from a lupin, a myoglobin from bovine and any combination thereof,
a nucleic acid sequence encoding at least one polypeptide involved in the biosynthesis of said globin polypeptide.

These aspects of the invention will be more fully understood in view of the following drawings, detailed description and non-limiting examples.

### BRIEF DESCRIPTION OF THE DRAWINGS

The accompanying drawings are included to further an understanding of the embodiments that are incorporated in and constitute a part of this specification. The drawings illustrate embodiments and together with the description serve to explain principles of embodiments. Other embodiments and many of the intended advantages of embodiments will be readily appreciated, as they become better understood by reference to the detailed description. The elements of the drawings are not necessarily to scale relative to each other.

FIG. 1 shows the sequence map of the pET24a-HLTev-VsLegH construct.

FIG. 2 shows the sequence map of the pET24a-HLTev-LlLegH construct.

FIG. 3 shows the protein expression of VsLegH (SEQ ID NO: 2) and HLTev-VsLegH (SEQ ID NO: 7), using either E. coli C41(DE3) or BL21(DE3). E. coli C41(DE3) gave better results as strain for the expression of both VsLegH (SEQ ID NO: 2) and HLTev-VsLegH (SEQ ID NO: 7).

FIG. 4 shows protein expression of HLTev-LlLegH (SEQ ID NO: 9), using either E. coli C41(DE3) or BL21(DE3). E. coli C41(DE3) gave better results as strain for the expression of HLTev-LlLegH (SEQ ID NO: 9). Addition of ALA and Fe(II) improved the heme incorporation into LlLegH.

FIG. 5 shows the comparison of HLTev-VsLegH (SEQ ID NO: 7) expression in SB and in 2×TY medium.

FIG. 6 shows the comparison of HLTev-LlLegH (SEQ ID NO: 9) expression in SB and in 2×TY medium.

FIG. 7 shows the elution profile of HLTev-VsLegH (SEQ ID NO: 7) expressed in E. coli C41(DE3).

FIG. 8 shows the elution profile of HLTev-VsLegH (SEQ ID NO: 7) expressed in E. coli BL21(DE3).

FIG. 9 shows the elution profile of HLTev-LlLegH (SEQ ID NO: 9) expressed in E. coli C41(DE3).

FIG. 10 shows the elution profile of HLTev-LlLegH (SEQ ID NO: 9) expressed in E. coli BL21(DE3).

FIG. 11 shows purified HLTev-VsLegH (SEQ ID NO: 7), expressed in E. coli C41(DE3).

FIG. 12 shows purified HLTev-LlLegH (SEQ ID NO: 9), expressed in E. coli C41(DE3) and BL21(DE3).

FIG. 13 shows purified HLTev-VsLegH (SEQ ID NO: 7), expressed in E. coli C41(DE3) and BL21(DE3). HLTev-VsLegH (SEQ ID NO: 7) has 254 amino acids, with a calculated M_{w} of 27408.02 and a theoretical pI of 5.02.

FIG. 14 shows purified HLTev-LlLegH (SEQ ID NO: 9), expressed in E. coli C41(DE3) and BL21(DE3). HLTev-LlLegH has 263 amino acids, with a calculated M_{w} of 28589.44 and a theoretical pI of 4.99.

FIG. 15 shows the UV-Vis spectra of purified HLTev-VsLegH (SEQ ID NO: 7), expressed in E. coli C41(DE3) and BL21(DE3).

FIG. 16 shows the UV-Vis spectra of purified HLTev-LlLegH (SEQ ID NO: 9), expressed in E. coli C41(DE3) and BL21(DE3). The p and a peaks seemed to merge together, indicating a potential substrate binding in the protein cavity.

FIG. 17 show the plasmid map of pET24a-HLTev-VsLegH.

FIG. 18 show the plasmid map of pET24a-HLTev-LlLegH.

FIG. 19 shows the sequence map of the pYES2-ACMVsLegH construct.

FIG. 20 shows the sequence map of the pYES2-ACMLlLegH construct.

FIG. 21 shows the sequence map of the pYES2-ACMBtMyg construct.

FIG. 22 shows the plasmid map of pYES2-ACMVsLegH.

FIG. 23 shows the plasmid map of pYES2-ACMLlLegH.

FIG. 24 shows the plasmid map of pYES2-ACMBtMyg.

FIG. 25 shows that the primer sets (SKIK-VsLegH-F|pYES2-R; SEQ ID NO: 13 and SEQ ID NO: 19) and (FBA-VsLegH-F|pYES2-R; SEQ ID NO: 14 and SEQ ID NO: 19) were used to introduce SKIK tag (SEQ ID NO: 12) and FBA tag (SEQ ID NO: 11) to the N-terminus of VsLegH (SEQ ID NO: 2), respectively.

FIG. 26 shows that the primer sets (SKIK-LlLegH-F|pYES2-R; SEQ ID NO: 15 and SEQ ID NO: 19) and (FBA-LlLegH-F|pYES2-R; SEQ ID NO: 16 and SEQ ID NO: 19) were used to introduce SKIK tag (SEQ ID NO: 12) and FBA tag (SEQ ID NO: 11) to the N-terminus of LlLegH (SEQ ID NO: 4), respectively.

FIG. 27 shows that the primer sets (SKIK-BtMyg-F|pYES2-R; SEQ ID NO: 17 and SEQ ID NO: 19) and (FBA-BtMyg-F|pYES2-R; SEQ ID NO: 18 and SEQ ID NO: 19) were used to introduce SKIK tag (SEQ ID NO: 12) and FBA tag (SEQ ID NO: 11) to the N-terminus of BtMyg (SEQ ID NO: 20), respectively.

FIG. 28 shows the plasmid map of pYES2-FBA-VsLegH.

FIG. 29 shows the plasmid map of pYES2-SKIK-VsLegH.

FIG. 30 shows the globin-expressing plasmids were transformed into S. cerevisiae INVSc1 cells.

FIG. 31 shows globin-expressing plasmids and heme-overexpressing plasmids (H3 or H3H2H12) were co-transformed into S. cerevisiae INVSc1 cells.

FIG. 32 shows protein expression of BtMyg (SEQ ID NO: 20) and VsLegH (SEQ ID NO: 2), using S. cerevisiae INVSc1 cells.

FIG. 33 shows protein expression of FBA-VsLegH (SEQ ID NO: 21) and SKIK-VsLegH (SEQ ID NO: 22), using S. cerevisiae INVSc1 cells.

FIG. 34 shows protein expression of FBA-VsLegH (SEQ ID NO: 21) and SKIK-VsLegH (SEQ ID NO: 22), using S. cerevisiae INVSc1 cells harbouring heme-overexpressing plasmid (H3 or H3H2H12).

FIG. 35 shows cell extract of S. cerevisiae INVSc1 cells, expressing heme biosynthesis gene(s) and FBA-VsLegH (SEQ ID NO: 21) or SKIK-VsLegH (SEQ ID NO: 22).

FIG. 36 shows SDS-PAGE analysis of the cell extract of S. cerevisiae INVSc1 cells, expressing heme biosynthesis gene(s) and FBA-VsLegH (SEQ ID NO: 21) or SKIK-VsLegH (SEQ ID NO: 22).

FIG. 37 shows UV-Vis spectra of the cell extract of S. cerevisiae INVSc1 cells, expressing heme biosynthesis gene(s) and FBA-VsLegH (SEQ ID NO: 21) or SKIK-VsLegH (SEQ ID NO: 22).

FIG. 38 shows a comparison of aggregation propensity of VsLegH (circles) and LlLegH (rectangles). The analysis was performed using Aggrescan.

FIG. 39 shows the Aggrescan analysis of LaLegH2, LaLegH1, LlLegH1 and LalLegH, compared to VsLegH and LlLegH.

FIG. 40 shows the protein model of LaLegH1, created using SWISS-MODEL.

FIG. 41 shows the protein model of LlLegH1, created using SWISS-MODEL.

FIG. 42 shows the recombinant protein expression of LaLegH1 in E. coli, using super broth-based autoinduction medium (SB-AIM) and 2×TY medium induced with IPTG.

FIG. 43 shows the recombinant protein expression of LlLegH1 in E. coli, using super broth-based autoinduction medium (SB-AIM) and 2×TY medium induced with IPTG.

FIG. 44 shows a comparison of protein expression level/heme incorporation of LaLegH1 and LlLegH1.

FIG. 45 shows the vector map of pTTB2-HLTev-VsLegH (SEQ ID NO: 47).

FIG. 46 shows the recombinant protein expression of HLTev-VsLegH in Bacillus subtilis TEA strain in LB or 2×TY, with or without ALA supplementation.

FIG. 47 shows the recombinant protein expression of HLTev-LlLegH in Bacillus subtilis TEA strain in LB or 2×TY, with or without ALA supplementation.

FIG. 48 shows the result returned when the protein sequence of VsLegH was used to BLAST against the Vigna subterranea genome.

FIG. 49 shows the sequence alignment of Vs001352g0011.1 (VsLegH, SEQ ID NO: 2), Vs001352g0009.1 (VsLegH9; SEQ ID NO: 35), Vs001352g0010.1 (VsLegH10, SEQ ID NO: 36) and Vs108178g0061.1 (VsLegH61; SEQ ID NO: 37). H62 and H93, the two residues responsible for heme binding, are conserved among all mentioned 4 sequences.

FIG. 50 shows the protein expression of VsLegH9 and VsLegH61 in Escherichia coli.

FIG. 51 shows the protein expression of VsLegH10 in Escherichia coli.

FIG. 52 shows the sequence alignment between bacterial hemoglobin from Vitreoscilla stercoraria (SEQ ID NO: 38; P04252_vhb) and leghemolobin from Vigna subterranea (SEQ ID NO: 2; VsLegH).

FIG. 53 shows the sequence alignment between bacterial hemoglobin from Vitreoscilla stercoraria (SEQ ID NO: 38; P04252_vhb) and leghemolobin from Glycine max (GmLegH) (SEQ ID NO: 32).

### DETAILED DESCRIPTION OF THE INVENTION

The following language and descriptions of certain preferred embodiments of the present invention are provided in order to further an understanding of the principles of the present invention. However, it will be understood that no limitations of the present invention are intended, and that further alterations, modifications, and applications of the principles of the present invention are also included.

In a first aspect, the present invention provides a method of producing globin polypeptide recombinantly, comprising:
transforming a host cell with an expression vector comprising one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof, wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

The term “globin polypeptide” as used herein and in the context of the present invention means any protein or polypeptide of a globin. Globins are a superfamily of heme-containing globular proteins, involved in binding and/or transporting oxygen. These proteins all incorporate the globin fold, a series of eight alpha helical segments. Two prominent members include myoglobin and hemoglobin. Both of these proteins reversibly bind oxygen via a heme prosthetic group.

In the present invention, the term “polypeptides” refers to peptides and proteins, whose length is about ten amino acids or longer. Polypeptides are ordinarily derived from organisms, but are not particularly limited thereto, and, for example, they may be composed of an artificially designed sequence. They may also be any of naturally derived polypeptides, synthetic polypeptides, recombinant polypeptides, or such. Additionally, fragments of the above-mentioned polypeptides are also included in the polypeptides of the present invention.

However, the globin polypeptide or protein produced by any of the methods of the present invention is directed to specific leghemoglobins or myoglobins, namely leghemoglobin from a Vigna plant, a leghemoglobin from a lupin, a myoglobin from bovine and any combination thereof. However, the globin polypeptide or protein produced by any of the methods of the present invention may also be a bacterial hemoglobin, preferably a bacterial hemoglobin having at least 70% sequence identity with the bacterial hemoglobin of Vitreoscilla stercoraria.

Leghemoglobin, also called leghemoglobin or legoglobin, is an oxygen carrier and hemoprotein found in the nitrogen-fixing root nodules of leguminous plants. It is produced by these plants in response to the roots being colonized by nitrogen-fixing bacteria, termed rhizobia, as part of the symbiotic interaction between plant and bacterium: roots not colonized by Rhizobium do not synthesize leghemoglobin. Leghemoglobin has close chemical and structural similarities to hemoglobin, and, like hemoglobin, is red in colour. Leghemoglobin is shown to buffer the concentration of free oxygen in the cytoplasm of infected plant cells to ensure the proper function of root nodules. That being said, nitrogen fixation is an extremely energetically costly process, so aerobic respiration, which necessitates high oxygen concentration, is necessary in the cells of the root nodule. Leghemoglobin maintains a free oxygen concentration that is low enough to allow nitrogenase to function, but a high enough total oxygen concentration (free and bound to leghemoglobin) for aerobic respiration. Leghemoglobins are monomeric proteins with a mass around 16 kDa, and are structurally similar to myoglobin. One leghemoglobin protein consists of a heme bound to an iron, and one polypeptide chain (the globin). Similar to myoglobin and hemoglobin, the iron of heme is found in its ferrous state in vivo, and is the moiety that binds oxygen. Leghemoglobin has a slow oxygen dissociation rate, similar to myoglobin. Like myoglobin and hemoglobin, leghemoglobin has a high affinity for carbon monoxide. Heme groups are the same in all known leghemoglobins, but the amino acid sequence of the globin differs slightly depending on bacterial strain and legume species. Even within one leguminous plant, multiple isoforms of leghemoglobins can exist. These often differ in oxygen affinity, and help meet the needs of a cell in a particular environment within the nodule.

Myoglobin is a well known iron- and oxygen-binding protein found in the skeletal muscle tissue of vertebrates in general and in almost all mammals. Myoglobin belongs to the globin superfamily of proteins, and as with other globins, consists of eight alpha helices connected by loops. Myoglobin contains 154 amino acids and a porphyrin ring with an iron at its center. A proximal histidine group (His-93) is attached directly to iron, and a distal histidine group (His-64) hovers near the opposite face. The distal imidazole is not bonded to the iron, but is available to interact with the substrate O₂. This interaction encourages the binding of O₂, but not carbon monoxide (CO), which still binds about 240× more strongly than O₂. The binding of O₂ causes substantial structural change at the Fe center, which shrinks in radius and moves into the center of the N4 pocket.

Vitreoscilla hemoglobin (VHb) is a type of hemoglobin found in the gram-negative aerobic bacterium Vitreoscilla. VHb is the best understood of all bacterial hemoglobins, and is attributed to play a number of functions. Its main role is likely the binding of oxygen at low concentrations and its direct delivery to the terminal respiratory oxidase(s), such as cytochrome o. It is also involved in the delivery of oxygen to oxygenases, detoxification of NO by converting it to nitrate, and sensing oxygen concentrations and passing this signal to transcription factors. It has a peroxidase-like activity and effectively eliminates autoxidation-derived H₂O₂, which is a cause of heme degradation and iron release.

Vigna is a genus of flowering plants in the legume family, Fabaceae, with a pantropical distribution. Thus, the term “Vigna plant” as used in the context of the present invention is a plant belonging to this genus. It includes some well-known cultivated species, including many types of beans. Some are former members of the genus Phaseolus. Vigna differs from Phaseolus in biochemistry and pollen structure, and in details of the style and stipules. Vigna is also commonly confused with the genus Dolichos, but the two differ in stigma structure.

“Lupin” or “lupin plant”, also commonly known as lupinus or lupine, as used within the context of the present invention, means a plant belonging to the genus of flowering plants in the legume family Fabaceae.

The term “bovine” as used herein and in the context of the present invention means of, relating to, or resembling a ruminant mammal of the bovid subfamily Bovinae, such as a cow, ox, or buffalo, especially one in the genus Bos. The biological subfamily Bovinae includes a diverse group of 10 genera of medium to large-sized ungulates, including domestic cattle, bison, African buffalo, the water buffalo, and the four-horned and spiral-horned antelopes.

The term “recombinant” as used herein refers to non-naturally modified or engineered nucleic acids, host cells transfected with foreign nucleic acids, or by manipulation of isolated DNA and transformation of host cells. It is used to describe non-naturally expressed polypeptides. “Recombinant” is a term that specifically encompasses DNA molecules constructed in vitro using genetic engineering techniques, and an adjective for describing a molecule, construct, vector, cell, polypeptide, or polynucleotide. The use of the term “recombinant” as specifically excludes naturally occurring molecules.

“Host cell” means generally any cell (prokaryotic or eukaryotic) transformed to contain the vector. According to the present invention, the host cell is a bacterial cell or a yeast cell. Preferred bacterial host cells include Escherichia coli, Bacillus subtilis or Lactococcus lactis. Preferred host cells include yeast cells, in particular, Saccharomyces, Pichia, Hansenula, Schizosaccharomyces, yeasts of the genus Kleiberomices, Yarrowia, and Candida. Preferred exemplary yeast species include S. cerevisiae, Hansenula polymorpha, Kleiberomyces lactis, Pichia pastoris, Schizosaccharomyces pombe, and Yarrowia ripolitica. A particularly preferred yeast host cell is S. cerevisiae.

The term “transformation” or “transforming” generally refers to an artificial (i.e., practitioner-controlled) method of introducing genetic material into a cell or phage without being limited to the method of insertion. Numerous methods are well-known to a person skilled in the art in this regard. In particular, the term “transformant” means a transformed host cell, e.g., adapted.

The term “cell culture” or “culturing (host) cell”, as used in the present invention, is generally regarded as a technique, by which cells are cultivated outside a living organism under controlled conditions (e.g., temperature, pH, nutrient, and waste levels). The most widely used cell-culture practice nowadays is to culture cells using multiwell microplates or Petri dishes as culture vessels.

The term “recovering”, as used in the present invention, means any process well known to the person skilled in the art, which allows the regaining of the produced polypeptide.

In one embodiment of the method of producing globin polypeptide recombinantly, the method comprises:
transforming a host cell with an expression vector comprising
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof,
wherein the host cell is selected from yeast or bacteria and wherein the yeast host cell is not a methylotropic yeast host cell,
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising one or more nucleic acid sequence(s) encoding said globin polypeptide, wherein said globin polypeptide is/are leghemoglobin from a Vigna plant,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is/are leghemoglobin from a Vigna plant,
wherein the host cell is a bacterial host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is/are leghemoglobin from a Vigna plant,
wherein the host cell is a yeast host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising one or more nucleic acid sequence(s) encoding said globin polypeptide, wherein said globin polypeptide has/have a sequence identity of at least 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Vigna subterranea,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Vigna subterranea,
wherein the host cell is a bacterial host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising one or more nucleic acid sequence(s) encoding said globin polypeptide, wherein said globin polypeptide has/have a sequence identity of at least 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Vigna subterranea,
wherein the host cell is a yeast host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is/are leghemoglobin from a lupin,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is/are leghemoglobin from a lupin,
wherein the host cell is a bacterial host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is/are leghemoglobin from a lupin,
wherein the host cell is a yeast host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Lupinus luteus,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Lupinus luteus,
wherein the host cell is a bacterial host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Lupinus luteus,
wherein the host cell is a yeast host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of myoglobin from Bos taurus,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of myoglobin from Bos taurus,
wherein the host cell is a bacterial host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of myoglobin from Bos taurus,
wherein the host cell is a yeast host cell;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is a bacterial hemoglobin having at least 70% sequence identity with the bacterial hemoglobin of Vitreoscilla stercoraria,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide has/have a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of bacterial hemoglobin of Vitreoscilla stercoraria,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

The percentage of sequence homology or sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10-3) including the propeptide sequences, preferably using the wild type protein scaffold as reference in a pairwise comparison. It is calculated as the percentage of numbers of “positives” (homologous amino acids) indicated as result in the BLASTP program output divided by the total number of amino acids selected by the program for the alignment. Further, for example, BLAST (https://blast.ncbi.nlm.nih.gov/Blast.cgi) can be applied to search for biological sequences sharing similarity (determination of sequence homology or sequence identity), e.g. with VsLegH, LlLegH and BtMyg, preforming the search against the ‘non-redundant protein sequences (nr)’ database.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is leghemoglobin from Vigna subterranea,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide comprises or consists of the protein sequence set forth in SEQ ID NO: 2,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is leghemoglobin from Lupinus luteus,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide comprises or consists of the protein sequence set forth in SEQ ID NO: 4,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is myoglobin from Bos taurus,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide comprises or consists of the protein sequence set forth in SEQ ID NO: 20,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide is bacterial hemoglobin of Vitreoscilla stercoraria,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment, the present invention is directed to a method of producing globin polypeptide recombinantly, comprising:
transforming a host cell with an expression vector comprising
one or more nucleic acid sequence(s) encoding said globin polypeptide, wherein said globin polypeptide comprises or consists of the protein sequence set forth in SEQ ID NO: 38,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

In one embodiment of the method of producing globin polypeptide recombinantly, the one or more nucleic acid sequence(s) encode(s) a leghemoglobin from a Vigna plant, preferably a Vigna plant selected from the group consisting of Vigna ambacensis, Vigna angivensis, Vigna filicaulis, Vigna friesiorum, Vigna gazensis, Vigna hosei, Vigna luteola, Vigna membranacea, Vigna monantha, Vigna racemosa, Vigna subterranea, and Vigna unguiculata, more preferably from Vigna subterranea.

Thus, in one more preferred embodiment of the present invention, the Vigna plant is Vigna subterranea.

Vigna subterranea (also known by its common names: Bambara nut, Bambara groundnut, Bambara-bean, Congo goober, earth pea, ground-bean, or hog-peanut) is a member of the family Fabaceae. The plant is originated in West Africa (the Bambara people are found in southern Mali, Guinea, Burkina Faso and Senegal). Vigna subterranea ripens its pods underground, much like the peanut (also called a groundnut).

In one embodiment of the method of the present invention, the Vigna plant is not Vigna radiata.

In one further embodiment of the method of the present invention, the Vigna plant is not Vigna unguiculata.

In one further embodiment of the method of producing globin polypeptide recombinantly, the one or more nucleic acid sequence(s) encode(s) a leghemoglobin from a lupin, preferably from a lupin selected from the group consisting of Lupinus albus, Lupinus angustifolius, Lupinus micranthus, Lupinus luteus, Lupinus hispanicus, Lupinus cosentinii, Lupinus digitatus, Lupinus princei, Lupinus pilosus, Lupinus palaestinus, Lupinus atlanticus, Lupinus mutabilis, Lupinus texensis, and Lupinus nootkatensis, more preferably Lupinus luteus.

Lupinus luteus is known as annual yellow-lupin, European yellow lupin or yellow lupin. It is native to the Mediterranean region of Southern Europe. It occurs on mild sandy and volcanic soils in mining belts. As a wild plant, it is widespread over the coastal area in the western part of the Iberian Peninsula, Morocco, Tunisia, and Algeria, on the islands of Corsica, Sardinia and Sicily and in Southern Italy.

In one embodiment of the method of producing globin polypeptide recombinantly, the one or more nucleic acid sequence(s) encode(s) a myoglobin from bovine, preferably a myoglobin from Bos taurus, Bos primigenius, Bos javanicus, Bos gaurus, Bos frontalis, Bos grunniens, Bos mutus, and Bos sauveli, more preferably from Bos taurus. In one embodiment of the method of producing globin polypeptide recombinantly, the one or more nucleic acid sequence(s) encode(s) myoglobin from Bos taurus.

In one embodiment of the method of producing globin polypeptide recombinantly, the one or more nucleic acid sequence(s) encode(s) a bacterial hemoglobin, preferably a bacterial hemoglobin having at least 70% sequence identity with the bacterial hemoglobin of Vitreoscilla stercoraria, more preferably wherein the one or more nucleic acid sequence(s) encode(s) the bacterial hemoglobin of Vitreoscilla stercoraria, of Vitreoscilla sp. HG1, or of the Vitreoscilla sp strain C1.

Also, in one embodiment of the method of producing globin polypeptide recombinantly, the host cell is a bacterial host cell, preferably a bacterial host cell selected from the group consisting of Escherichia coli, Bacillus subtilis and Lactococcus lactis. In one preferred embodiment of the method of producing globin polypeptide recombinantly, the host cell is Escherichia coli. In one preferred embodiment of the method of producing globin polypeptide recombinantly, the host cell is Bacillus subtilis. In one preferred embodiment of the method of producing globin polypeptide recombinantly, the host cell is Lactococcus lactis.

In another embodiment of the method of producing globin polypeptide recombinantly, the host cell is a yeast host cell, preferably a yeast host cell of the genus Saccharomyces, Pichia, Candida, Torulopsis or Hansenula, more preferably of Saccharomyces cerevisiae. In one preferred embodiment of the method of producing globin polypeptide recombinantly, the host cell is Saccharomyces cerevisiae.

It is also preferred for the method of producing globin polypeptide recombinantly of the present invention, that said globin polypeptide is/are heterologous with respect to said host cell.

Additionally, in one embodiment of the method of producing globin polypeptide recombinantly, it is preferred that said one or more nucleic acid sequence(s) is/are under regulation of a promoter or tandem promoters functional in bacteria or yeast. In one embodiment, it is more preferred that said promoter is a bacterial promoter. In one embodiment, it is even more preferred that the bacterial promoter is selected from the group consisting of the araBAD promoter, lac promoter, lacUV5 promoter, phoA promoter, pL promoter, pR promoter, rhaBAD promoter, Sp6 promoter, T3 promoter, T5 promoter, T7 promoter, T7lac promoter, tac promoter, tet promoter, trc promoter and the trp promoter, most preferably the T7lac promoter. In one embodiment, it is preferred that said promoter is a yeast promoter. In one embodiment, it is even more preferred that the yeast promoter is selected from the group consisting of the GAL1 promoter, GAL10 promoter, GALL promoter, GALS promoter, CTR1 promoter, CTR3 promoter, CUP1 promoter, CYC1 promoter, MET25 promoter, the promoter of glyceraldehyde 3-phosphate dehydrogenase (GPD), the promoter of alcohol dehydrogenase 1 (ADH1), the promoter of transcriptional elongation factor EF-1α (TEF1), the promoter of transcriptional elongation factor EF-1α (TEF2), the promoter of phosphoglycerate kinase (PGK1), the promoter of triose phosphate isomerase (TP11), the promoter of hexose transporter (HXT7), the promoter of pyruvate kinase 1 (PYK1), and the promoter of triose phosphate dehydrogenase (TDH3), most preferably the GAL1 promoter. The term “promoter” or “promoter sequence” means a DNA sequence, which initiates and directs the transcription of a gene into an RNA transcript in cells.

In one embodiment of the method of producing globin polypeptide recombinantly, the one or more nucleic acid sequence(s) encoding the globin polypeptide comprise(s) or consist(s) of a sequence selected from the group consisting of SEQ ID NO: 1, SEQ ID NO: 3, SEQ ID NO: 5, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46. The present invention also comprises fragments of these mentioned sequences. In one embodiment of the method of producing globin polypeptide recombinantly, the one or more nucleic acid sequence(s) encoding the globin polypeptide comprise(s) or consist(s) of a sequence selected from the group consisting of SEQ ID NO: 1, SEQ ID NO: 3, and SEQ ID NO: 5. In one preferred embodiment of the method of producing globin polypeptide recombinantly, the one or more nucleic acid sequence comprises or consists of a sequence set forth in SEQ ID NO: 1. In one preferred embodiment of the method of producing globin polypeptide recombinantly, the one or more nucleic acid sequence comprises or consists of a sequence set forth in SEQ ID NO: 3. In one preferred embodiment of the method of producing globin polypeptide recombinantly, the one or more nucleic acid sequence comprises or consists of a sequence set forth in SEQ ID NO: 5. In one preferred embodiment of the method of producing globin polypeptide recombinantly, the one or more nucleic acid sequence comprises or consists of a sequence set forth in SEQ ID NO: 39. In one preferred embodiment of the method of producing globin polypeptide recombinantly, the one or more nucleic acid sequence comprises or consists of a sequence set forth in SEQ ID NO: 40. In one preferred embodiment of the method of producing globin polypeptide recombinantly, the one or more nucleic acid sequence comprises or consists of a sequence set forth in SEQ ID NO: 41. In one preferred embodiment of the method of producing globin polypeptide recombinantly, the one or more nucleic acid sequence comprises or consists of a sequence set forth in SEQ ID NO: 42. In one preferred embodiment of the method of producing globin polypeptide recombinantly, the one or more nucleic acid sequence comprises or consists of a sequence set forth in SEQ ID NO: 43. In one preferred embodiment of the method of producing globin polypeptide recombinantly, the one or more nucleic acid sequence comprises or consists of a sequence set forth in SEQ ID NO: 44. In one preferred embodiment of the method of producing globin polypeptide recombinantly, the one or more nucleic acid sequence comprises or consists of a sequence set forth in SEQ ID NO: 45. In one preferred embodiment of the method of producing globin polypeptide recombinantly, the one or more nucleic acid sequence comprises or consists of a sequence set forth in SEQ ID NO: 46. In one preferred embodiment of the method of producing globin polypeptide recombinantly, the one or more nucleic acid sequence encodes a globin polypeptide, which comprises or consists of a sequence set forth in SEQ ID NO: 2. In one preferred embodiment of the method of producing globin polypeptide recombinantly, the one or more nucleic acid sequence encodes a globin polypeptide, which comprises or consists of a sequence set forth in SEQ ID NO: 4. In one preferred embodiment of the method of producing globin polypeptide recombinantly, the one or more nucleic acid sequence encodes a globin polypeptide, which comprises or consists of a sequence set forth in SEQ ID NO: 20. In one preferred embodiment of the method of producing globin polypeptide recombinantly, the one or more nucleic acid sequence encodes a globin polypeptide, which comprises or consists of a sequence set forth in SEQ ID NO: 23. In one preferred embodiment of the method of producing globin polypeptide recombinantly, the one or more nucleic acid sequence encodes a globin polypeptide, which comprises or consists of a sequence set forth in SEQ ID NO: 25. In one preferred embodiment of the method of producing globin polypeptide recombinantly, the one or more nucleic acid sequence encodes a globin polypeptide, which comprises or consists of a sequence set forth in SEQ ID NO: 31. In one preferred embodiment of the method of producing globin polypeptide recombinantly, the one or more nucleic acid sequence encodes a globin polypeptide, which comprises or consists of a sequence set forth in SEQ ID NO: 32. In one preferred embodiment of the method of producing globin polypeptide recombinantly, the one or more nucleic acid sequence encodes a globin polypeptide, which comprises or consists of a sequence set forth in SEQ ID NO: 33. In one preferred embodiment of the method of producing globin polypeptide recombinantly, the one or more nucleic acid sequence encodes a globin polypeptide, which comprises or consists of a sequence set forth in SEQ ID NO: 34. In one preferred embodiment of the method of producing globin polypeptide recombinantly, the one or more nucleic acid sequence encodes a globin polypeptide, which comprises or consists of a sequence set forth in SEQ ID NO: 35. In one preferred embodiment of the method of producing globin polypeptide recombinantly, the one or more nucleic acid sequence encodes a globin polypeptide, which comprises or consists of a sequence set forth in SEQ ID NO: 36. In one preferred embodiment of the method of producing globin polypeptide recombinantly, the one or more nucleic acid sequence encodes a globin polypeptide, which comprises or consists of a sequence set forth in SEQ ID NO: 37. In one preferred embodiment of the method of producing globin polypeptide recombinantly, the one or more nucleic acid sequence encodes a globin polypeptide, which comprises or consists of a sequence set forth in SEQ ID NO: 38.

In a further aspect, the present invention provides a method of producing globin polypeptide with a cell-free translation system, comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
translating the one or more nucleic acid sequence(s) with the cell-free translation system, and
recovering the globin polypeptide from the cell-free translation system.

In one embodiment, the present invention provides a method of producing globin polypeptide with a cell-free translation system, comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine and any combination thereof,
translating the one or more nucleic acid sequence(s) with the cell-free translation system, and
recovering the globin polypeptide from the cell-free translation system.

In one embodiment of this method of producing globin polypeptide with a cell-free translation system, the cell-free translation system is a bacterial cell-free system or a yeast cell-free system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a Vigna plant,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a lupin,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are myoglobin from bovine,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are bacterial hemoglobin having at least 70% sequence identity with the bacterial hemoglobin of Vitreoscilla stercoraria,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a Vigna plant,
translating the one or more nucleic acid sequence(s) with the cell-free translation system, wherein the cell-free translation system is a bacterial cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a lupin,
translating the one or more nucleic acid sequence(s) with the cell-free translation system, wherein the cell-free translation system is a bacterial cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are myoglobin from bovine,
translating the one or more nucleic acid sequence(s) with the cell-free translation system, wherein the cell-free translation system is a bacterial cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are bacterial hemoglobin of Vitreoscilla stercoraria,
translating the one or more nucleic acid sequence(s) with the cell-free translation system, wherein the cell-free translation system is a bacterial cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a Vigna plant,
translating the one or more nucleic acid sequence(s) with the cell-free translation system, wherein the cell-free translation system is a yeast cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are leghemoglobin from a lupin,
translating the one or more nucleic acid sequence(s) with the cell-free translation system, wherein the cell-free translation system is a yeast cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are myoglobin from bovine,
translating the one or more nucleic acid sequence(s) with the cell-free translation system, wherein the cell-free translation system is a yeast cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein the globin polypeptide is/are bacterial hemoglobin of Vitreoscilla stercoraria,
translating the one or more nucleic acid sequence(s) with the cell-free translation system, wherein the cell-free translation system is a yeast cell-free system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein said globin polypeptide has/have a sequence identity of at least 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Vigna subterranea,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein said globin polypeptide has/have a sequence identity of at least 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of leghemoglobin from Lupinus luteus,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein said globin polypeptide has/have a sequence identity of at least 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of myoglobin from Bos taurus,
translating the one or more nucleic acid sequence(s) with the cell-free translation system, and
recovering the globin polypeptide from the cell-free translation system.

In one embodiment of the method of producing globin polypeptide with a cell-free translation system, the method comprises:
Providing one or more nucleic acid sequence(s) encoding the globin polypeptide, wherein said globin polypeptide has/have a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the protein sequence of bacterial hemoglobin of Vitreoscilla stercoraria,
translating the one or more nucleic acid sequence(s) with the cell-free translation system, and
recovering the globin polypeptide from the cell-free translation system.

In one embodiment of this method of producing globin polypeptide with a cell-free translation system of the present invention, the Vigna plant is not Vigna radiata.

In one further embodiment of this method of producing globin polypeptide with a cell-free translation system of the present invention, the Vigna plant is not Vigna unguiculata.

In a further aspect, the present invention provides a food product, preferably a meat substitute food product, comprising: one or more globin protein(s), wherein said globin protein(s) is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria and any combination thereof. In one embodiment, the present invention provides a food product, preferably a meat substitute food product, comprising: one or more globin protein(s), wherein said globin protein(s) is/are selected from the group consisting of a leghemoglobin from a Vigna plant, a leghemoglobin from a lupin, a myoglobin from bovine and any combination thereof.

Preferably, the globin protein(s) is/are leghemoglobin from a Vigna plant.

Preferably, the globin protein(s) is/are leghemoglobin from a lupin.

Preferably, in one further embodiment, the globin protein(s) is/are myoglobin from bovine.

Preferably, the globin protein(s) is/are bacterial hemoglobin of Vitreoscilla stercoraria.

In a further embodiment of the food product, preferably the meat substitute food product, it further comprises:
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning.

Thus, in one embodiment, the food product, preferably the meat substitute food product, of the present invention comprises:
one or more globin protein(s), wherein said globin protein is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning.

Thus, in one embodiment, the food product, preferably the meat substitute food product, of the present invention comprises:
one or more globin protein(s), wherein said globin protein is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning.

Thus, in one embodiment, the food product, preferably the meat substitute food product, of the present invention comprises:
one or more globin protein(s), wherein said globin protein is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optionally one or more cell based protein.

In a preferred embodiment, the one or more fibre(s) may be, for example, fibres from cane, Vigna species, or lupin species as defined above, e.g. Vigna ambacensis, Vigna angivensis, Vigna filicaulis, Vigna friesiorum, Vigna gazensis, Vigna hosei, Vigna luteola, Vigna membranacea, Vigna monantha, Vigna racemosa, Vigna subterranea, or Vigna unguiculata or e.g. Lupinus albus, Lupinus angustifolius, Lupinus micranthus, Lupinus luteus, Lupinus hispanicus, Lupinus cosentinii, Lupinus digitatus, Lupinus princei, Lupinus pilosus, Lupinus palaestinus, Lupinus atlanticus, Lupinus mutabilis, Lupinus texensis, and Lupinus nootkatensis. In one more preferred embodiment, the one or more fibre(s) is/are from a plant, more preferably a legume or a grain. More specifically, the one or more fibre may be from alfalfa, clover, beans, peas, chickpeas, lentils, lupins, mesquite, carob, soybeans, peanuts, or tamarind.

In a preferred embodiment, the one or more carbohydrate(s) may be, for example, wheat flour, potato starch, or Bambara groundnut flour. In one more preferred embodiment, the one or more carbohydrate(s) is/are from a plant, more preferably from a legume or a grain. More specifically, the one or more carbohydrate(s) may be from alfalfa, clover, beans, peas, chickpeas, lentils, lupins, mesquite, carob, soybeans, peanuts, or tamarind.

In a preferred embodiment, the one or more fats may be, for example, shea butter or coconut oil. In one more preferred embodiment, the one or more fat(s) is/are from non-animals.

In a preferred embodiment, the one or more micronutrient(s), may be, for example, vitamins, or minerals. The micronutrient may be, for example, iron, zinc and/or vitamin A.

In one preferred embodiment, the one or more other proteins than the one or more globin protein(s) may be from a plant, even more preferably from a legume or a grain. More specifically, the one or more other proteins than the one or more globin protein(s) may be from alfalfa, clover, beans, peas, chickpeas, lentils, lupins, mesquite, carob, soybeans, peanuts, or tamarind.

In a preferred embodiment, the one or more flavors, yeast extracts, hydrolized vegetable proteins, herbs and/or seasoning may be, for example, salt or reaction flavors. In one preferred embodiment, the one or more flavors, yeast extracts, hydrolized vegetable proteins, herbs and/or seasoning and/or seasoning may be plant flavors and/or plant seasoning.

In one preferred embodiment, the present invention provides a food product, preferably a meat substitute food product, comprising:
one or more globin protein(s), wherein said globin protein is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals, and
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning,
one or more egg albumin,
one or more hydrocolloid,
optionally one or more mycoprotein, and
optionally one or more cell based protein.

In a preferred embodiment, the egg albumin may be, for example, ovalbumin or lactalbumin.

In a preferred embodiment, the hydrocolloid may be, for example, selected from the group consisting of pectin (E 440), gum arabic (E 414), guar gum (E 412), agar (E 406), carrageen (E 407), alginate (E 400-E 404) and xanthan (E 415).

In a preferred embodiment, the mycoprotein may be a high protein, high fibre, low fat food ingredient derived from fermentation of the filamentous fungus Fusarium venenatum.

In a preferred embodiment, the cell based protein may be, any protein that can be gained by cell-based protein expression. Such an expression allows for producing of high levels of recombinant protein production, using prokaryotic or eukaryotic cells. E. coli is a common host for such a method. The most popular method for E. coli cell-based recombinant protein expression uses a T7 expression host and an expression vector containing a T7 promoter.

In one preferred embodiment, the present invention provides a meat substitute food product, comprising:
one or more globin protein(s), wherein said globin protein is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals, and
one or more micronutrients,
one or more other proteins than the one or more globin protein(s), preferably one or more protein from a plant, more preferably one or more protein from a legume or a grain, and
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optionally one or more cell based protein.

In one preferred embodiment of the food product of the present invention, the one or more globin protein comprise(s) or consist(s) of a sequence set forth in SEQ ID NO: 2, SEQ ID NO: 4, SEQ ID NO: 23, SEQ ID NO: 25, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 38. The present invention also comprises fragments of these mentioned sequences.

In one preferred embodiment of the food product of the present invention, the globin polypeptide which is a leghemoglobin from a Vigna plant, more preferably a globin polypeptide, which has at least 82% sequence identity with leghemoglobin of Vigna subterranea. It is further preferred that the globin polypeptide is leghemoglobin of Vigna subterranea. It is even further preferred that the globin polypeptide comprises or consists of a sequence set forth in SEQ ID NO: 2.

In one embodiment of the food product of the present invention, the Vigna plant is not Vigna radiata.

In one further embodiment of the food product of the present invention, the Vigna plant is not Vigna unguiculata.

In one preferred embodiment of the food product of the present invention, the globin polypeptide is a leghemoglobin from a lupin, more preferably a globin polypeptide, which has at least 59% sequence identity with leghemoglobin of Lupinus luteus. It is further preferred that the globin polypeptide is the leghemoglobin of Lupinus luteus. It is even further preferred that the globin polypeptide comprises or consists of a sequence set forth in SEQ ID NO: 4.

In one preferred embodiment of the food product of the present invention, the globin polypeptide is a myoglobin from bovine, more preferably a globin polypeptide, which has at least 80% sequence identity with myoglobin of Bos taurus. It is further preferred that the globin polypeptide is the myoglobin of Bos taurus. It is even further preferred that the globin polypeptide comprises or consists of a sequence set forth in SEQ ID NO: 20.

In one preferred embodiment of the food product of the present invention, the globin polypeptide comprises or consists of a sequence set forth in SEQ ID NO: 23.

In one preferred embodiment of the food product of the present invention, the globin polypeptide comprises or consists of a sequence set forth in SEQ ID NO: 25.

In one preferred embodiment of the food product of the present invention, the globin polypeptide comprises or consists of a sequence set forth in SEQ ID NO: 31.

In one preferred embodiment of the food product of the present invention, the globin polypeptide comprises or consists of a sequence set forth in SEQ ID NO: 32.

In one preferred embodiment of the food product of the present invention, the globin polypeptide comprises or consists of a sequence set forth in SEQ ID NO: 33.

In one preferred embodiment of the food product of the present invention, the globin polypeptide comprises or consists of a sequence set forth in SEQ ID NO: 34.

In one preferred embodiment of the food product of the present invention, the globin polypeptide comprises or consists of a sequence set forth in SEQ ID NO: 35.

In one preferred embodiment of the food product of the present invention, the globin polypeptide comprises or consists of a sequence set forth in SEQ ID NO: 36.

In one preferred embodiment of the food product of the present invention, the globin polypeptide comprises or consists of a sequence set forth in SEQ ID NO: 37.

In one preferred embodiment of the food product of the present invention, the globin polypeptide is a bacterial hemoglobin having at least 70% sequence identity with the bacterial hemoglobin of Vitreoscilla stercoraria. It is further preferred that the globin polypeptide is the bacterial hemoglobin of Vitreoscilla stercoraria. It is even further preferred that the globin polypeptide comprises or consists of a sequence set forth in SEQ ID NO: 38.

The present invention provides with this food product a consumable comprising a specific meat-like aroma, appearance, flavour, texture and taste.

In a further aspect, the present invention provides a vector comprising:
a nucleic acid sequence encoding a transcriptional activator,
one or more nucleic acid sequence(s) encoding globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof; and
a nucleic acid sequence encoding one or more tag protein(s), preferably one or more tag protein(s) selected from the group consisting of Biotin-carboxy carrier protein (BCCP), Calmodulin, Chitin-binding domain (CBD), Glutathione-S-transferase (GST), HaloTag, Maltose-binding protein (MBP), Polyhistidine tag, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, N-utilization substance A (NusA), Peptidyl-prolyl cis-trans isomerase B (PpiB), Thioredoxin (Trx), and SUMO, more preferably the tag protein Polyhistidine-tag and/or the lipoyl domain from Bacillus stearothermophilus E2p.

In one embodiment, the present invention provides a vector comprising:
a nucleic acid sequence encoding a transcriptional activator,
one or more nucleic acid sequence(s) encoding globin polypeptide, wherein said globin polypeptide is/are selected from the group consisting of a leghemoglobin from a Vigna plant, a leghemoglobin from a lupin, a myoglobin from bovine and any combination thereof; and
a nucleic acid sequence encoding one or more tag protein(s), preferably one or more tag protein(s) selected from the group consisting of Biotin-carboxy carrier protein (BCCP), Calmodulin, Chitin-binding domain (CBD), Glutathione-S-transferase (GST), HaloTag, Maltose-binding protein (MBP), Polyhistidine tag, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, N-utilization substance A (NusA), Peptidyl-prolyl cis-trans isomerase B (PpiB), Thioredoxin (Trx), and SUMO, more preferably the tag protein Polyhistidine-tag and/or the lipoyl domain from Bacillus stearothermophilus E2p.

In one preferred embodiment of the vector of the present invention, the one or more nucleic acid sequence(s) encodes a globin polypeptide, which is a leghemoglobin from a Vigna plant, more preferably a globin polypeptide, which has at least 82% sequence identity with leghemoglobin of Vigna subterranea. It is further preferred that the one or more nucleic acid sequence(s) encodes a globin polypeptide, which is leghemoglobin of Vigna subterranea. It is even further preferred that the one or more nucleic acid sequence(s) encodes a globin polypeptide, which comprises or consists of a sequence set forth in SEQ ID NO: 2.

In one preferred embodiment of the vector of the present invention, the one or more nucleic acid sequence(s) encodes a globin polypeptide, which is a leghemoglobin from a lupin, more preferably a globin polypeptide, which has at least 59% sequence identity with leghemoglobin of Lupinus luteus. It is further preferred that the one or more nucleic acid sequence(s) encodes a globin polypeptide, which is the leghemoglobin of Lupinus luteus. It is even further preferred that the one or more nucleic acid sequence(s) encodes a globin polypeptide, which comprises or consists of a sequence set forth in SEQ ID NO: 4.

In one preferred embodiment of the vector of the present invention, the one or more nucleic acid sequence(s) encodes a globin polypeptide, which is a myoglobin from bovine, more preferably a globin polypeptide, which has at least 80% sequence identity with myoglobin of Bos taurus. It is further preferred that the one or more nucleic acid sequence(s) encodes a globin polypeptide, which is the myoglobin of Bos taurus. It is even further preferred that the one or more nucleic acid sequence(s) encodes a globin polypeptide, which comprises or consists of a sequence set forth in SEQ ID NO: 20.

In one preferred embodiment of the vector of the present invention, the globin polypeptide comprises or consists of a sequence set forth in SEQ ID NO: 23.

In one preferred embodiment of the vector of the present invention, the globin polypeptide comprises or consists of a sequence set forth in SEQ ID NO: 25.

In one preferred embodiment of the vector of the present invention, the globin polypeptide comprises or consists of a sequence set forth in SEQ ID NO: 31.

In one preferred embodiment of the vector of the present invention, the globin polypeptide comprises or consists of a sequence set forth in SEQ ID NO: 32.

In one preferred embodiment of the vector of the present invention, the globin polypeptide comprises or consists of a sequence set forth in SEQ ID NO: 33.

In one preferred embodiment of the vector of the present invention, the globin polypeptide comprises or consists of a sequence set forth in SEQ ID NO: 34.

In one preferred embodiment of the vector of the present invention, the globin polypeptide comprises or consists of a sequence set forth in SEQ ID NO: 35.

In one preferred embodiment of the vector of the present invention, the globin polypeptide comprises or consists of a sequence set forth in SEQ ID NO: 36.

In one preferred embodiment of the vector of the present invention, the globin polypeptide comprises or consists of a sequence set forth in SEQ ID NO: 37.

In one preferred embodiment of the vector of the present invention, the one or more nucleic acid sequence(s) encodes a globin polypeptide, which is a bacterial hemoglobin, preferably a bacterial hemoglobin having at least 70% sequence identity with the bacterial hemoglobin of Vitreoscilla stercoraria. It is further preferred that the one or more nucleic acid sequence(s) encodes a globin polypeptide, which is the bacterial hemoglobin of Vitreoscilla stercoraria. It is even further preferred that the one or more nucleic acid sequence(s) encodes a globin polypeptide, which comprises or consists of a sequence set forth in SEQ ID NO: 38.

The one or more tag(s)/tag protein(s) mentioned above may function as N-terminal tags, wherein they significantly help to increase protein expression level of the globin polypeptide or protein and assist protein purification respectively.

In the context of the present invention and as used herein, the terms “protein” and “polypeptide” can be used interchangeably.

In a further aspect, the present invention provides a system for recombinant globin polypeptide production, comprising:
a bacterial or yeast cell or a cell-free translation system; and
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof.

In a further aspect, the present invention provides a system for recombinant globin polypeptide production, comprising:
a bacterial or yeast cell or a cell-free translation system; and
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof.

In one preferred embodiment of the system of the present invention, the one or more nucleic acid sequence(s) encodes a globin polypeptide, which is a leghemoglobin from a Vigna plant, more preferably a globin polypeptide, which has at least 82% sequence identity with leghemoglobin of Vigna subterranea. It is further preferred that the one or more nucleic acid sequence(s) encode(s) a globin polypeptide, which is leghemoglobin of Vigna subterranea. It is even further preferred that the one or more nucleic acid sequence(s) encode(s) a globin polypeptide, which comprises or consists of a sequence set forth in SEQ ID NO: 2.

In one preferred embodiment of the system of the present invention, the one or more nucleic acid sequence(s) encodes a globin polypeptide, which is a leghemoglobin from a lupin, more preferably a globin polypeptide, which has at least 59% sequence identity with leghemoglobin of Lupinus luteus. It is further preferred that the one or more nucleic acid sequence(s) encode(s) a globin polypeptide, which is the leghemoglobin of Lupinus luteus. It is even further preferred that the one or more nucleic acid sequence(s) encode(s) a globin polypeptide, which comprises or consists of a sequence set forth in SEQ ID NO: 4.

In one preferred embodiment of the system of the present invention, the one or more nucleic acid sequence(s) encodes a globin polypeptide, which is a myoglobin from bovine, more preferably a globin polypeptide, which has at least 80% sequence identity with myoglobin of Bos taurus. It is further preferred that the one or more nucleic acid sequence(s) encode(s) a globin polypeptide, which is the myoglobin of Bos taurus. It is even further preferred that the one or more nucleic acid sequence(s) encode(s) a globin polypeptide, which comprises or consists of a sequence set forth in SEQ ID NO: 20.

In one preferred embodiment of the system of the present invention, the globin polypeptide comprises or consists of a sequence set forth in SEQ ID NO: 23.

In one preferred embodiment of the system of the present invention, the globin polypeptide comprises or consists of a sequence set forth in SEQ ID NO: 25.

In one preferred embodiment of the system of the present invention, the globin polypeptide comprises or consists of a sequence set forth in SEQ ID NO: 31.

In one preferred embodiment of the system of the present invention, the globin polypeptide comprises or consists of a sequence set forth in SEQ ID NO: 32.

In one preferred embodiment of the system of the present invention, the globin polypeptide comprises or consists of a sequence set forth in SEQ ID NO: 33.

In one preferred embodiment of the system of the present invention, the globin polypeptide comprises or consists of a sequence set forth in SEQ ID NO: 34.

In one preferred embodiment of the system of the present invention, the globin polypeptide comprises or consists of a sequence set forth in SEQ ID NO: 35.

In one preferred embodiment of the system of the present invention, the globin polypeptide comprises or consists of a sequence set forth in SEQ ID NO: 36.

In one preferred embodiment of the system of the present invention, the globin polypeptide comprises or consists of a sequence set forth in SEQ ID NO: 37.

In one preferred embodiment of the system of the present invention, the one or more nucleic acid sequence(s) encodes a globin polypeptide, which is a bacterial hemoglobin, more preferably a globin polypeptide, which having at least 70% sequence identity with the bacterial hemoglobin of Vitreoscilla stercoraria. It is further preferred that the one or more nucleic acid sequence(s) encode(s) a globin polypeptide, which is the bacterial hemoglobin of Vitreoscilla stercoraria. It is even further preferred that the one or more nucleic acid sequence(s) encode(s) a globin polypeptide, which comprises or consists of a sequence set forth in SEQ ID NO: 38.

In another aspect, the present invention provides a cell comprising a recombinant expression vector or one or more recombinant nucleic acid molecule(s), comprising:
a nucleic acid sequence encoding a transcriptional activator,
one or more nucleic acid sequence(s) encoding a globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof, and
a nucleic acid sequence encoding at least one polypeptide involved in the biosynthesis of said globin polypeptide.

In one embodiment, the present invention provides a cell comprising a recombinant expression vector or one or more recombinant nucleic acid molecule(s), comprising:
a nucleic acid sequence encoding a transcriptional activator,
one or more nucleic acid sequence(s) encoding a globin polypeptide, wherein said globin polypeptide is/are selected from the group consisting of a leghemoglobin from a Vigna plant, a leghemoglobin from a lupin, a myoglobin from bovine and any combination thereof, and
a nucleic acid sequence encoding at least one polypeptide involved in the biosynthesis of said globin polypeptide.

In one preferred embodiment of the cell of the present invention, the one or more nucleic acid sequence(s) encodes a globin polypeptide, which is a leghemoglobin from a Vigna plant, more preferably a globin polypeptide, which has at least 82% sequence identity with the leghemoglobin of Vigna subterranea. It is further preferred that the one or more nucleic acid sequence(s) encode(s) (a) globin polypeptide, which is leghemoglobin of Vigna subterranea. It is even further preferred that the one or more nucleic acid sequence(s) encode(s) (a) globin polypeptide, which comprises or consists of a sequence set forth in SEQ ID NO: 2.

In one preferred embodiment of the cell of the present invention, the one or more nucleic acid sequence(s) encodes a globin polypeptide, which is a leghemoglobin from a lupin, more preferably a globin polypeptide, which has at least 59% sequence identity with leghemoglobin of Lupinus luteus. It is further preferred that the one or more nucleic acid sequence(s) encode(s) (a) globin polypeptide, which is the leghemoglobin of Lupinus luteus. It is even further preferred that the one or more nucleic acid sequence(s) encode(s) (a) globin polypeptide, which comprises or consists of a sequence set forth in SEQ ID NO: 4.

In one preferred embodiment of the cell of the present invention, the one or more nucleic acid sequence(s) encodes a globin polypeptide, which is a myoglobin from bovine, more preferably a globin polypeptide, which has at least 80% sequence identity with myoglobin of Bos taurus. It is further preferred that the one or more nucleic acid sequence(s) encode(s) (a) globin polypeptide, which is the myoglobin of Bos taurus. It is even further preferred that the one or more nucleic acid sequence(s) encode(s) (a) globin polypeptide, which comprises or consists of a sequence set forth in SEQ ID NO: 20.

In one preferred embodiment of the cell of the present invention, the globin polypeptide comprises or consists of a sequence set forth in SEQ ID NO: 23.

In one preferred embodiment of the cell of the present invention, the globin polypeptide comprises or consists of a sequence set forth in SEQ ID NO: 25.

In one preferred embodiment of the cell of the present invention, the globin polypeptide comprises or consists of a sequence set forth in SEQ ID NO: 31.

In one preferred embodiment of the cell of the present invention, the globin polypeptide comprises or consists of a sequence set forth in SEQ ID NO: 32.

In one preferred embodiment of the cell of the present invention, the globin polypeptide comprises or consists of a sequence set forth in SEQ ID NO: 33.

In one preferred embodiment of the cell of the present invention, the globin polypeptide comprises or consists of a sequence set forth in SEQ ID NO: 34.

In one preferred embodiment of the cell of the present invention, the globin polypeptide comprises or consists of a sequence set forth in SEQ ID NO: 35.

In one preferred embodiment of the cell of the present invention, the globin polypeptide comprises or consists of a sequence set forth in SEQ ID NO: 36.

In one preferred embodiment of the cell of the present invention, the globin polypeptide comprises or consists of a sequence set forth in SEQ ID NO: 37.

In one preferred embodiment of the cell of the present invention, the one or more nucleic acid sequence(s) encodes a globin polypeptide, which is a bacterial hemoglobin, more preferably a globin polypeptide, which has at least 70% sequence identity with the bacterial hemoglobin of Vitreoscilla stercoraria. It is further preferred that the one or more nucleic acid sequence(s) encode(s) (a) globin polypeptide, which is the bacterial hemoglobin of Vitreoscilla stercoraria. It is even further preferred that the one or more nucleic acid sequence(s) encode(s) (a) globin polypeptide, which comprises or consists of a sequence set forth in SEQ ID NO: 38.

The invention is further characterized by the following items:

Items:

1. Method of producing globin polypeptide recombinantly, comprising:
transforming a host cell with an expression vector comprising one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

2. The method of item 1, wherein the one or more nucleic acid sequence(s) encode(s) a leghemoglobin from a Vigna plant, preferably a Vigna plant selected from the group consisting of Vigna ambacensis, Vigna angivensis, Vigna filicaulis, Vigna friesiorum, Vigna gazensis, Vigna hosei, Vigna luteola, Vigna membranacea, Vigna monantha, Vigna racemosa, Vigna subterranea, and Vigna unguiculata, more preferably Vigna subterranea.

3. The method of item 1 or 2, wherein the one or more nucleic acid sequence(s) encode(s) a leghemoglobin from a lupin, preferably a lupin selected from the group consisting of Lupinus albus, Lupinus angustifolius, Lupinus micranthus, Lupinus luteus, Lupinus hispanicus, Lupinus cosentinii, Lupinus digitatus, Lupinus princei, Lupinus pilosus, Lupinus palaestinus, Lupinus atlanticus, Lupinus mutabilis, Lupinus texensis, and Lupinus nootkatensis, more preferably Lupinus luteus.

4. The method of any one of the previous items, wherein the one or more nucleic acid sequence(s) encode(s) a myoglobin from bovine, preferably a myoglobin from Bos taurus, Bos primigenius, Bos javanicus, Bos gaurus, Bos frontalis, Bos grunniens, Bos mutus, and Bos sauveli, more preferably Bos taurus.

5. The method of any one of the previous items, wherein the host cell is a bacterial host cell, preferably a bacterial host cell selected from the group consisting of Escherichia coli, Bacillus subtilis and Lactococcus lactis.

6. The method of any one of the previous items, wherein the host cell is a yeast host cell, preferably a yeast host cell of the genus Saccharomyces, Pichia, Candida, Torulopsis or Hansenula, more preferably of Saccharomyces cerevisiae.

7. The method of any one of the previous items, wherein said globin polypeptide is/are heterologous with respect to said host cell.

8. The method of any one of the previous items, wherein said one or more nucleic acid sequence(s) is/are under regulation of a promoter functional in bacteria or yeast, preferably wherein said promoter is a bacterial promoter, more preferably wherein the bacterial promoter is selected from the group consisting of the araBAD promoter, lac promoter, lacUV5 promoter, phoA promoter, pL promoter, pR promoter, rhaBAD promoter, Sp6 promoter, T3 promoter, T5 promoter, T7 promoter, T7lac promoter, tac promoter, tet promoter, trc promoter and the trp promoter, even more preferably the T7lac promoter; or, preferably wherein said promoter is a yeast promoter, more preferably wherein the yeast promoter is selected from the group consisting of the GAL1 promoter, GAL10 promoter, GALL promoter, GALS promoter, CTR1 promoter, CTR3 promoter, CUP1 promoter, CYC1 promoter, MET25 promoter, the promoter of glyceraldehyde 3-phosphate dehydrogenase (GPD), the promoter of alcohol dehydrogenase 1 (ADH1), the promoter of transcriptional elongation factor EF-1α (TEF1), the promoter of transcriptional elongation factor EF-1α (TEF2), the promoter of phosphoglycerate kinase (PGK1), the promoter of triose phosphate isomerase (TP11), the promoter of hexose transporter (HXT7), the promoter of pyruvate kinase 1 (PYK1), and the promoter of triose phosphate dehydrogenase (TDH3), even more preferably the GAL1 promoter.

9. The method of any one of the previous items, wherein the one or more nucleic acid sequence(s) encoding the globin polypeptide comprise(s) or consist(s) of a sequence selected from the group consisting of SEQ ID NO: 1, SEQ ID NO: 3, and SEQ ID NO: 5.

10. Method of producing globin polypeptide with a cell-free translation system,
comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine and any combination thereof,
translating the one or more nucleic acid sequence(s) with the cell-free translation system, and
recovering the globin polypeptide from the cell-free translation system.

11. The method of item 10, wherein the cell-free translation system is a bacterial cell-free system or a yeast cell-free system.

12. A food product, preferably a meat substitute food product, comprising:
one or more globin protein(s), wherein said globin protein is/are selected from the group consisting of a leghemoglobin from a Vigna plant, a leghemoglobin from a lupin, a myoglobin from bovine and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals, and
one or more micronutrients,
one or more other proteins than the one or more globin protein(s), preferably one or more protein from a plant, more preferably one or more protein from a legume or a grain,
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optonally one or more cell based protein.

13. The food product of item 12, wherein the one or more globin protein comprise(s) or consist(s) of a sequence set forth in SEQ ID NO: 2 or SEQ ID NO: 4.

14. A vector comprising:
a nucleic acid sequence encoding a transcriptional activator,
one or more nucleic acid sequence(s) encoding globin polypeptide, wherein said globin polypeptide is/are selected from the group consisting of a leghemoglobin from a Vigna plant, a leghemoglobin from a lupin, a myoglobin from bovine and any combination thereof;
a nucleic acid sequence encoding a tag protein, preferably a tag protein selected from the group consisting of Biotin-carboxy carrier protein (BCCP), Calmodulin, Chitin-binding domain (CBD), Glutathione-S-transferase (GST), HaloTag, Maltose-binding protein (MBP), Polyhistidine tag, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, N-utilization substance A (NusA), Peptidyl-prolyl cis-trans isomerase B (PpiB), Thioredoxin (Trx), and SUMO, more preferably the tag protein Polyhistidine-tag and/or the lipoyl domain from Bacillus stearothermophilus E2p.

15. System for recombinant globin polypeptide production, comprising:
a bacterial or yeast cell or a cell-free translation system; and
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine and any combination thereof.

16. A cell comprising a recombinant expression vector or one or more recombinant nucleic acid molecule(s), comprising:
a nucleic acid sequence encoding a transcriptional activator,
one or more nucleic acid sequence(s) encoding a globin polypeptide, wherein said globin polypeptide is/are selected from the group consisting of a leghemoglobin from a Vigna plant, a leghemoglobin from a lupin, a myoglobin from bovine and any combination thereof,
a nucleic acid sequence encoding at least one polypeptide involved in biosynthesis of said globin polypeptide.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES

### I. Bacterial Production System

### Example 1: Molecular Cloning of Genes Encoding VsLegH and LlLegH

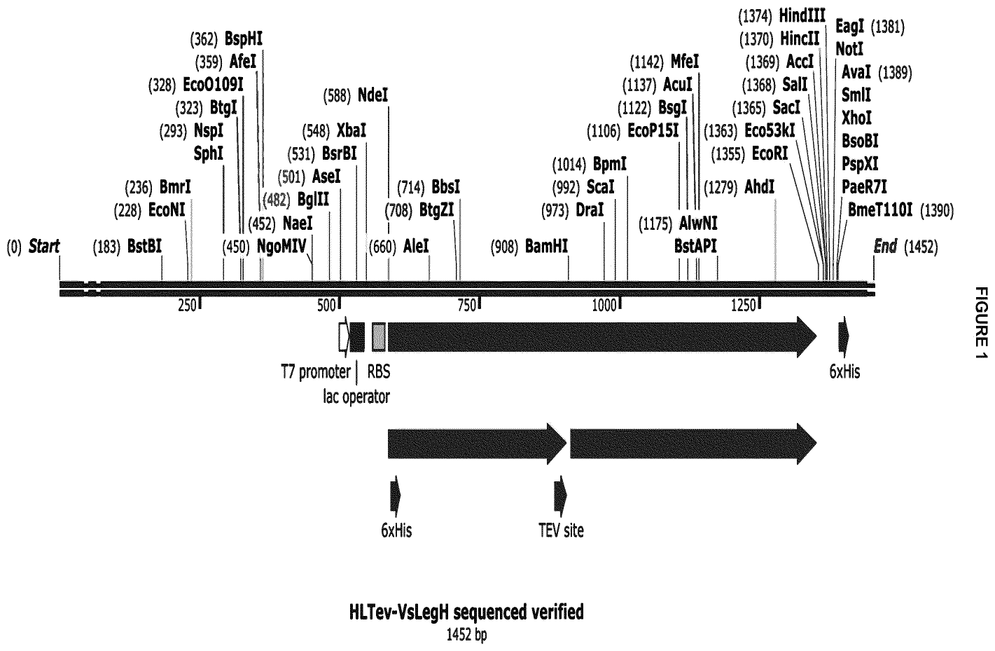

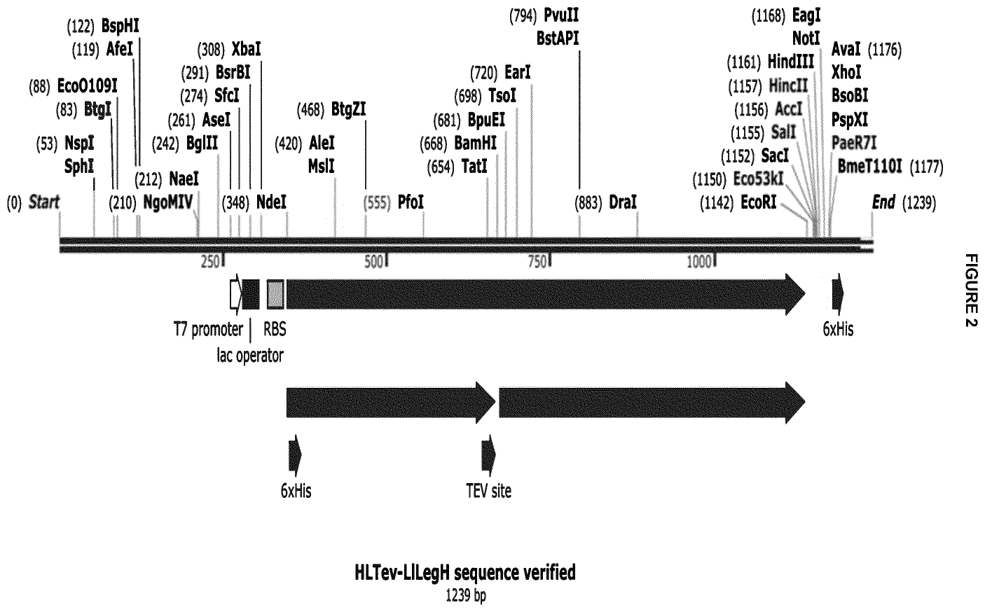

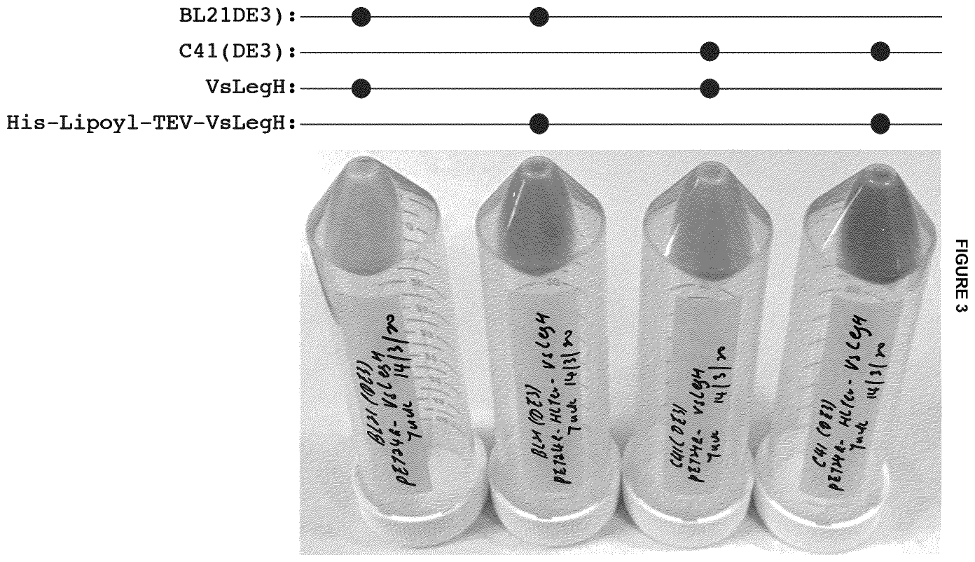

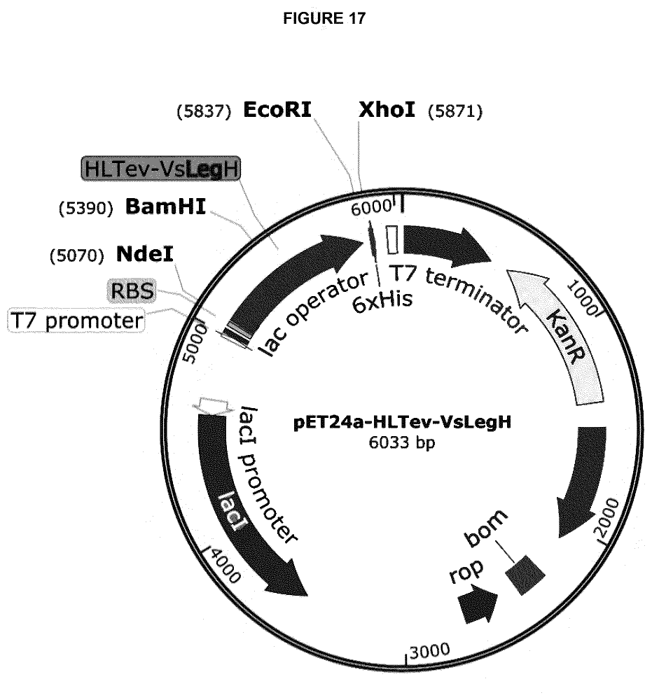

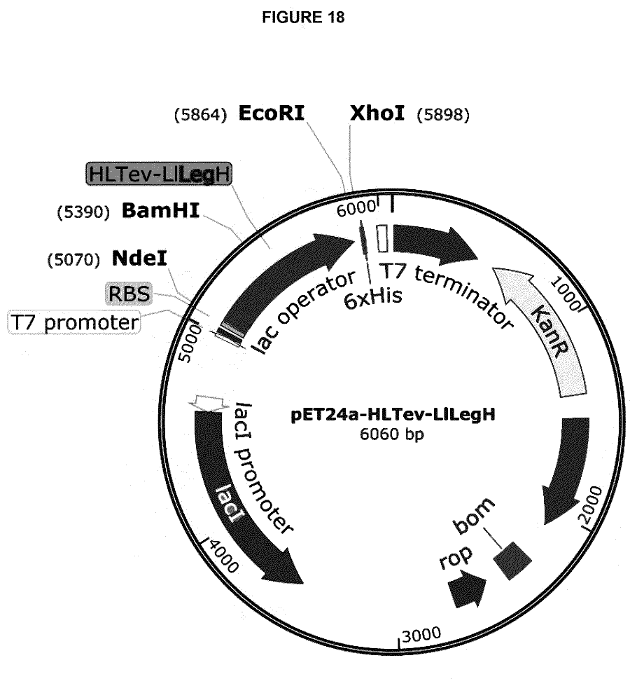

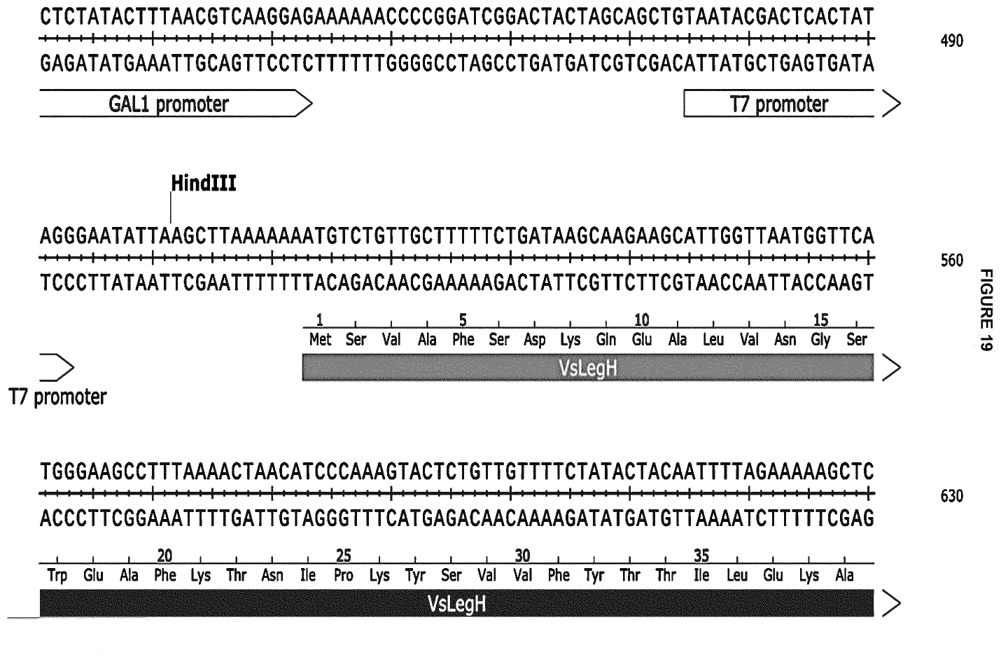

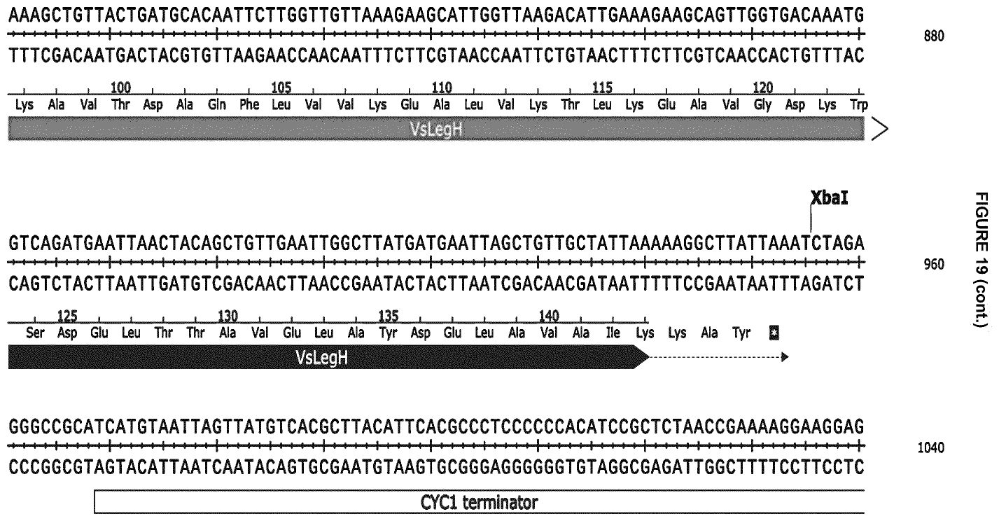

Genes encoding VsLegH (SEQ ID NO: 1) and LlLegH (SEQ ID NO: 2) were cloned into the pET24a-HLTev vector using BamHI and EcoRI sites, as shown in FIGS. 1 and 2, respectively. The DNA sequences and the protein sequences of HLTev-VsLegH and of HLTev-LlLegH were provided in SEQ ID NO: 1, SEQ ID NO: 2, SEQ ID NO: 3 and SEQ ID NO: 4. The full plasmid maps of pET24a-HLTev-VsLegH and pET24a-HLTev-VsLegH are shown in FIGS. 17 and 18.

The HLTev tag was used to serve three purposes: (a) to improve protein yield, (b) to assist downstream processing (i.e., protein purification), and (c) to increase protein stability. Other protein tags, like Biotin-carboxy carrier protein (BCCP), Calmodulin, Chitin-binding domain (CBD), Glutathione-S-transferase (GST), HaloTag, Maltose-binding protein (MBP), Polyhistidine, SBP-tag, Strep-tag II, Twin-Strep-tag, AFV₁₋₉₉ from Acidianus filamentous virus (AFV), Aggregation-resistant protein (SlyD), AmpC-type β-lactamase (Bla), Disulphide isomerase I (DsbA), Elongation factor Ts (Tsf), Fasciola hepatica antigen (Fh8), Lipoyl domain from Bacillus stearothermophilus E2p, N-utilization substance A (NusA), Peptidyl-prolyl cis-trans isomerase B (PpiB), Thioredoxin (Trx) or SUMO, will likely provide similar benefits, when used singly or in combinations. The tag can be removed via a proteolytic digestion. T7lac promoter was used for the protein expression. Other bacterial promoters, like araBAD, lac, lacUV5, phoA, pL, pR, rhaBAD, Sp6, T3, T5, T7, T7lac, tac, tet, trc, trp may be also applied singly or in combinations. T7lac was used herein.

### Example 2: Small-Scale Protein Expression

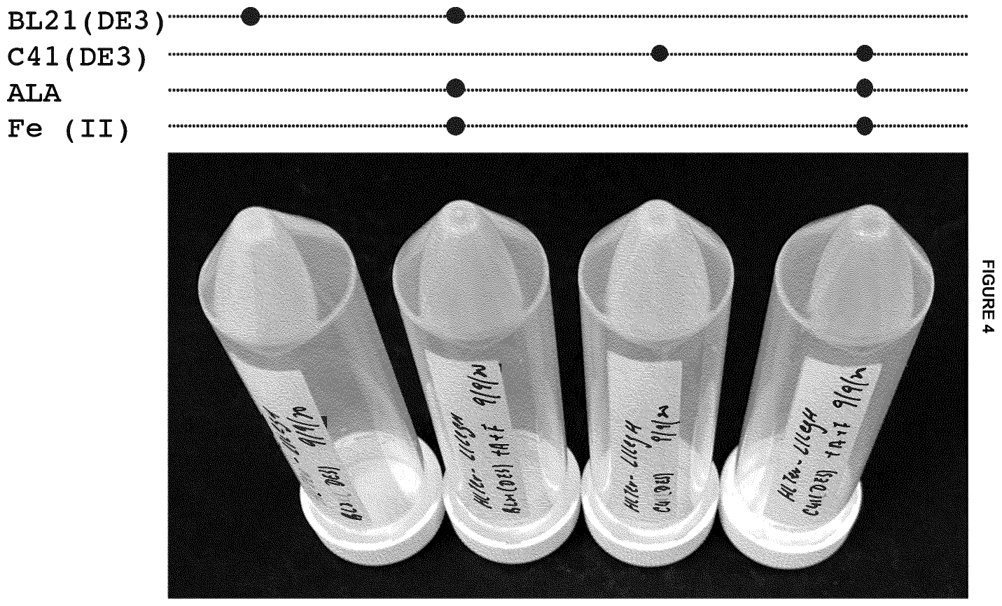

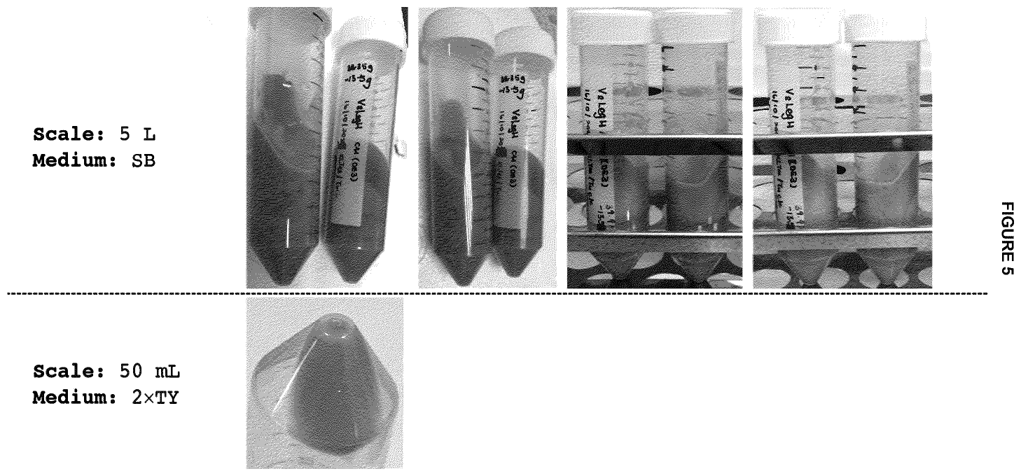

Plasmid was freshly transformed into either E. coli C41(DE3) or BL21(DE3). An overnight culture (5 mL of 2×TY medium, supplemented with 50 pg/mL of kanamycin; medium composition was for 2×TY medium, per L: 16 g tryptone, 10 g yeast extract, 5 g NaCl) was prepared from a single colony. Fifty mL of 2×TY medium, supplemented with 50 pg/mL of kanamycin, was inoculated with the overnight culture to a starting OD₆₀₀ of 0.1. The culture was incubated in an incubator shaker at 37° C. and 200 rpm. Bacterial growth was monitored by measuring OD₆₀₀ values. When OD₆₀₀ reached 0.5-0.6, 1 mM IPTG was added to induce protein expression and temperature was lowered to 30° C. Cells were harvested after 24 hours. For the expression of HLTev-LlLegH (SEQ ID NO: 9), the expression medium was supplemented with 0.5 mM ALA and 5 μM FeCl₂. Protein expression was confirmed by the reddish colour of the cell pellet, as shown in FIGS. 3 and 4. Cell pellets were stored at −80° C.

### Example 3: Large-Scale Protein Expression

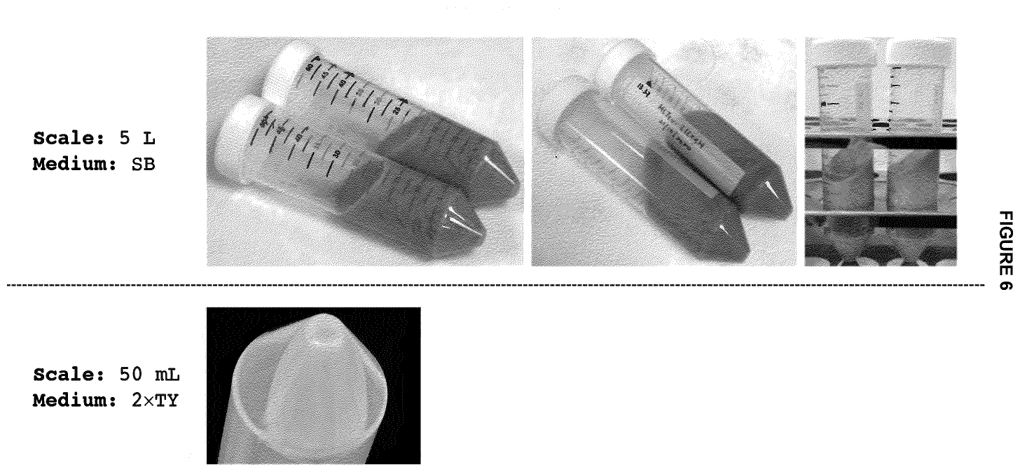

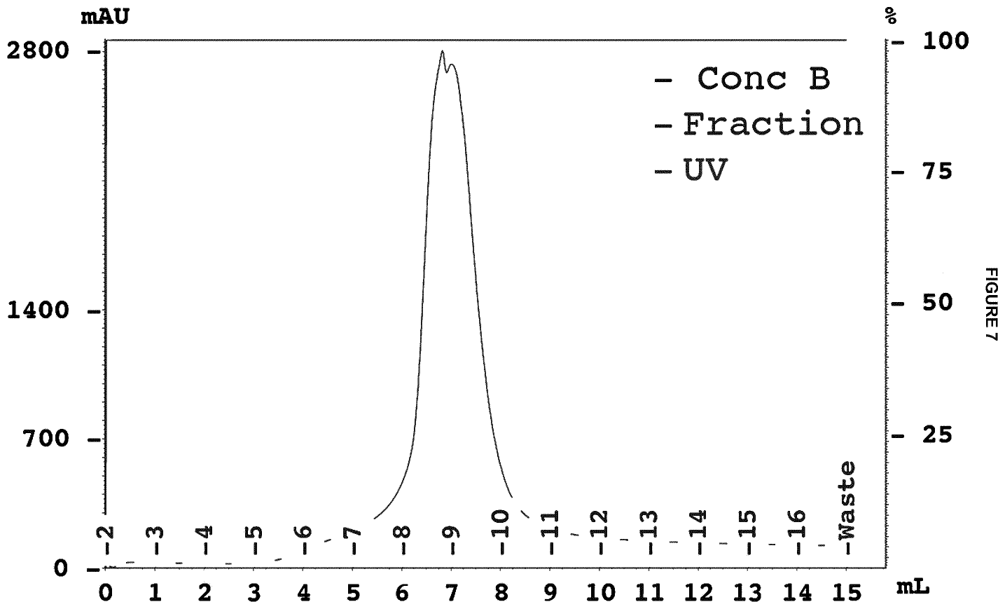

Plasmids were freshly transformed into E. coli C41(DE3). A tube culture (1 mL of 2×TY medium, supplemented with 50 pg/mL of kanamycin; medium composition was for 2×TY medium, per L: 16 g tryptone, 10 g yeast extract, 5 g NaCl) was prepared from a single colony and incubated at 37° C., 200 rpm for 6 hours. A 250-mL Erlenmeyer flask containing 50 mL of fresh 2×TY medium, supplemented with 50 pg/mL of kanamycin, was inoculated with 500 pL of the tube culture, and incubated at 30° C., 200 rpm for overnight. One-litre Erlenmeyer flasks containing 400 mL of SB (medium composition of SB medium per L: 35 g tryptone, 20 g yeast extract, 5 g NaCl), supplemented with 50 pg/mL of kanamycin, was inoculated with 2 mL of overnight culture. The culture was incubated in an incubator shaker at 37° C. and 200 rpm. Bacterial growth was monitored by measuring OD₆₀₀ values. When OD₆₀₀ reached 0.5-0.6, 1 mM IPTG was added to induce protein expression and temperature was lowered to 30° C. Cells were harvested after 24 hours. For the expression of HLTev-LlLegH (SEQ ID NO: 9), the expression medium was supplemented with 0.5 mM ALA and 5 μM FeCl₂. Protein expression was confirmed by the reddish colour of the cell pellet, as shown in FIGS. 5 an 6. Cell pellets were stored at −80° C. The use of SB improved the yield of heme-incorporated VsLegH and LlLegH significantly.

### Example 4: Protein Extraction

The cell pellet from a 50-mL culture was resuspended in 20 mL of buffer A (50 mM sodium phosphate, 300 mM NaCl, 10 mM imidazole, pH 8.0), supplemented with 10 μg/mL of lysozyme, 10 μg/mL of DNase, and 10 μg/mL of RNase. Cells were disrupted by a 5-min pulse sonication (Sonics; on time 15 sec, off time 45 sec, amplitude 70%). After sonication, cell debris was removed by centrifugation at 8500 rpm and 4° C. for 15 min.

### Example 5: Protein Purification

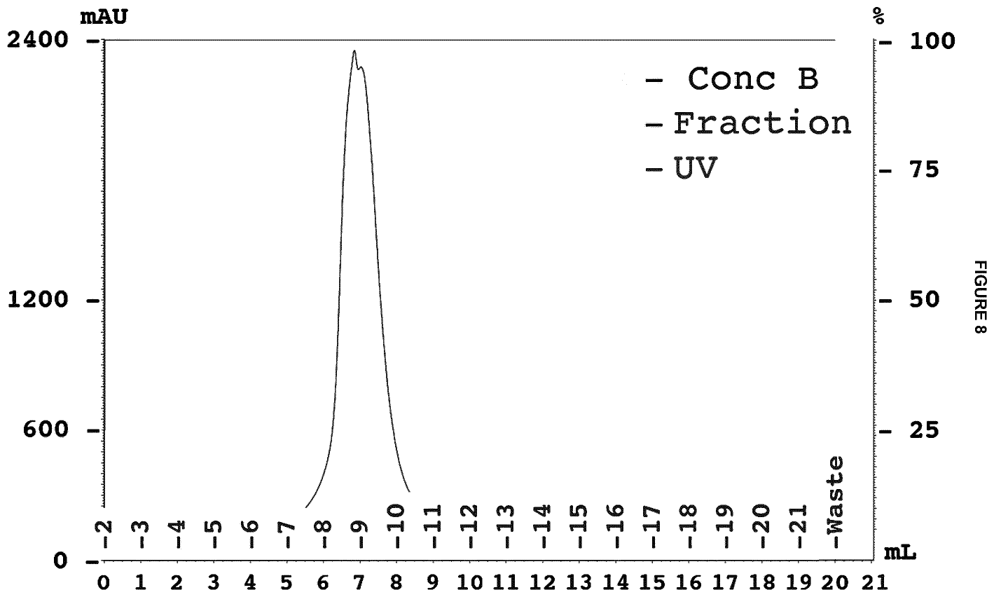

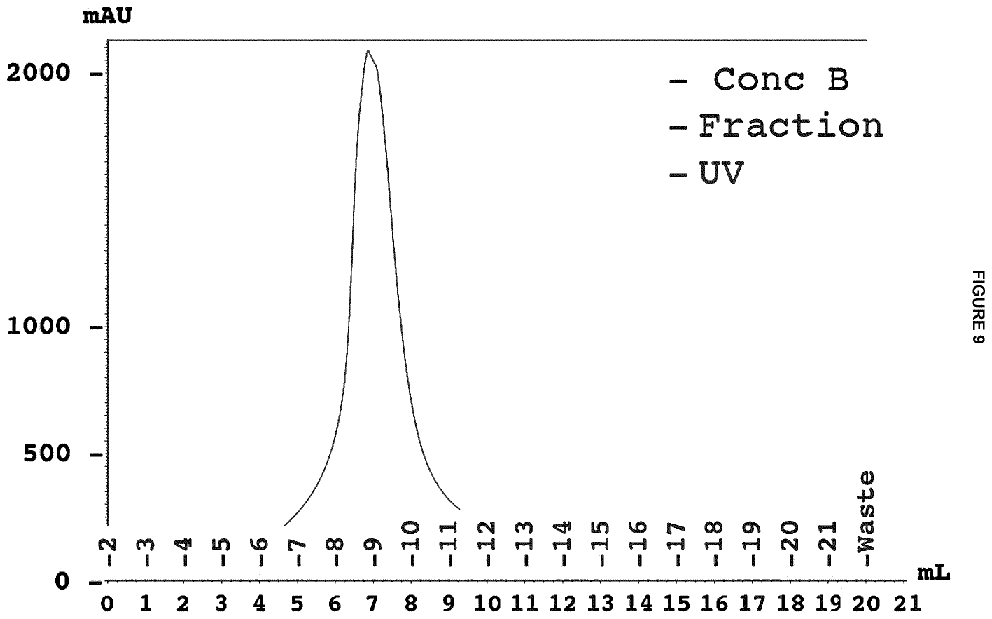

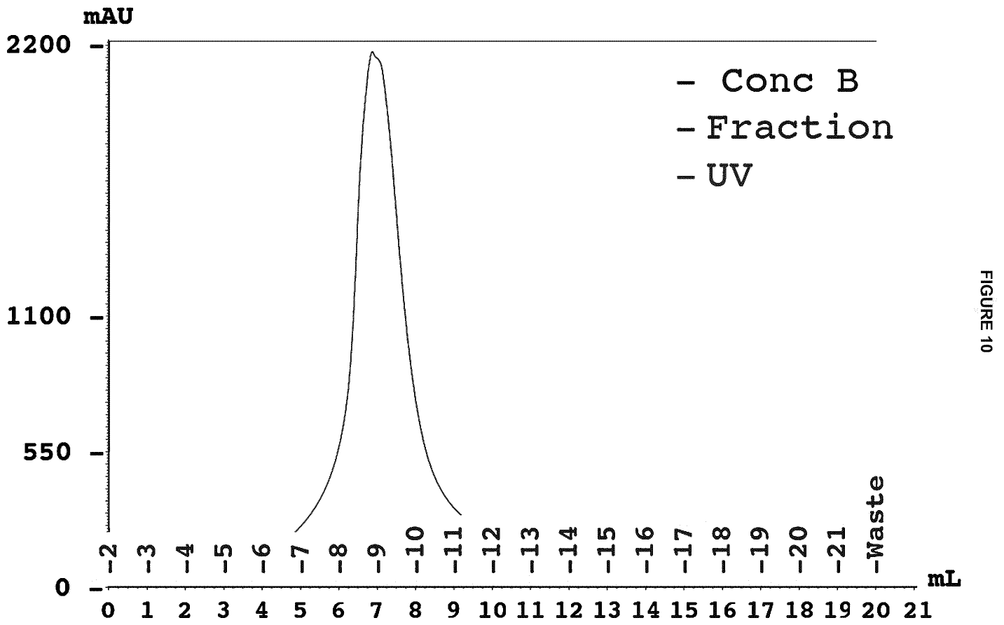

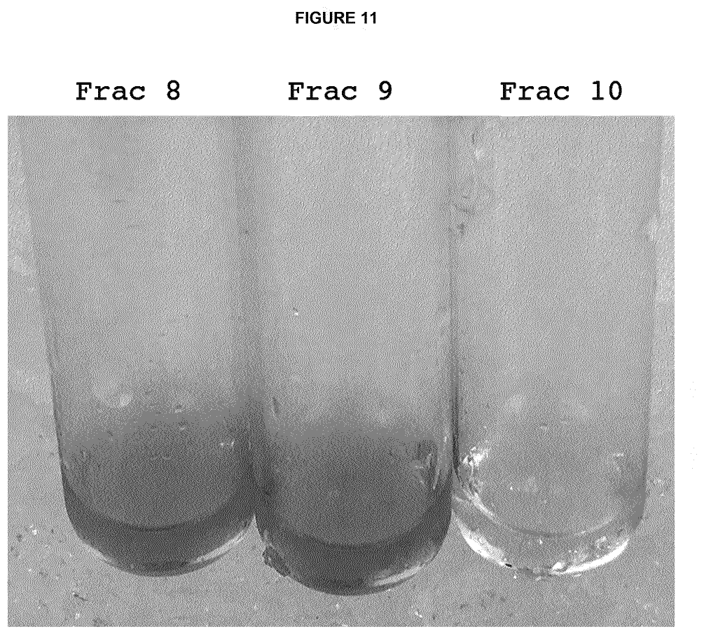

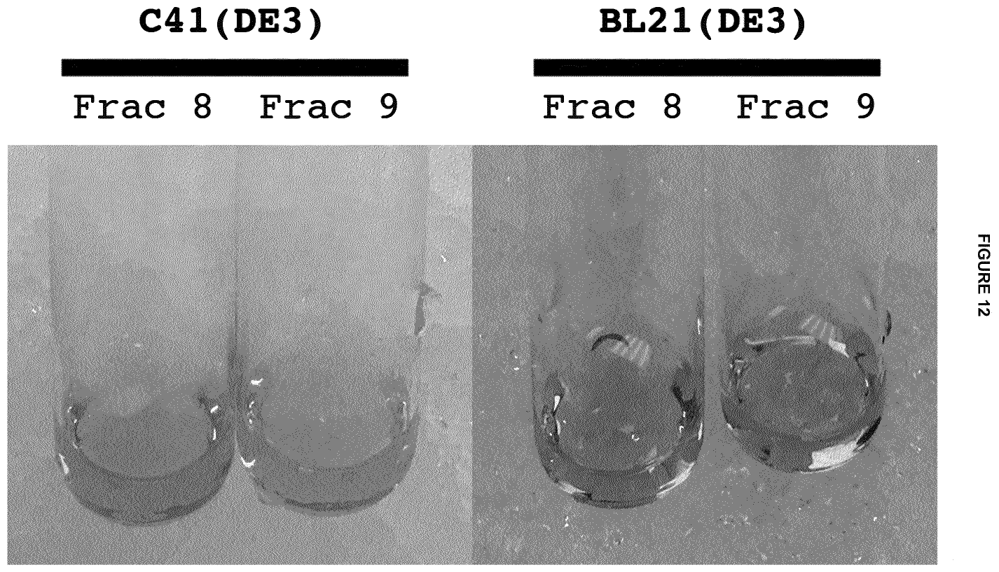

Protein purification was conducted using an ÄKTA Pure system (Cytiva). A 5-mL HisTrap™ HP column (Cytiva) was washed with 5 column volumes (CVs) of buffer B (50 mM sodium phosphate, 300 mM NaCl, 250 mM imidazole, pH 8.0), and equilibrated with 5 CVs of buffer A. Protein extract was filtered using a 0.45 μm syringe filter and loaded onto the equilibrated column using a sample pump. After sample loading, the column was washed with 5 CVs of buffer A. Protein was eluted using 100% (v/v) buffer B in reverse flow, and collected in fractions (1 mL/fraction) using a fraction collector. Elution profiles were shown in FIGS. 7-10. Purified LegH was reddish in colour (FIGS. 11 and 12).

### Example 6: SDS-PAGE

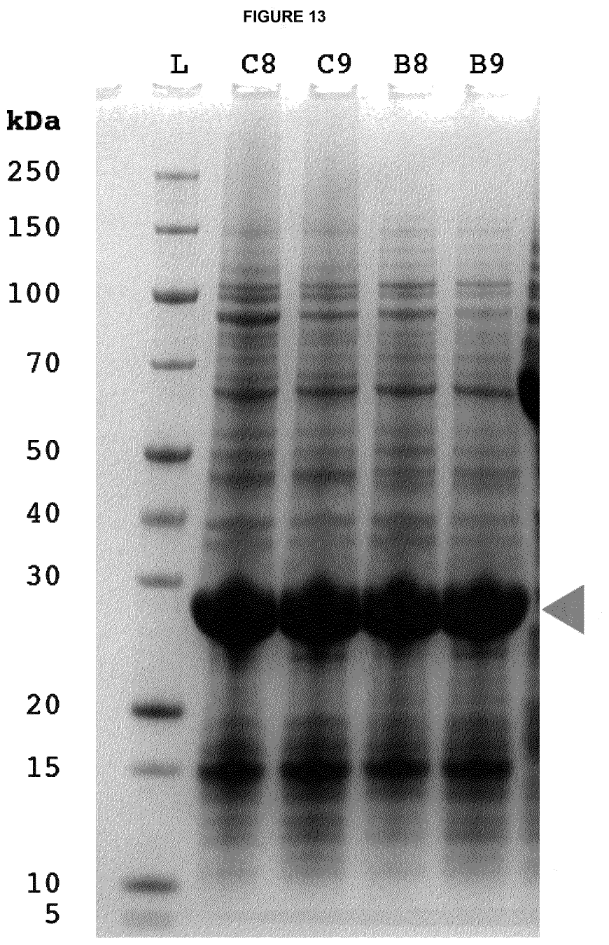

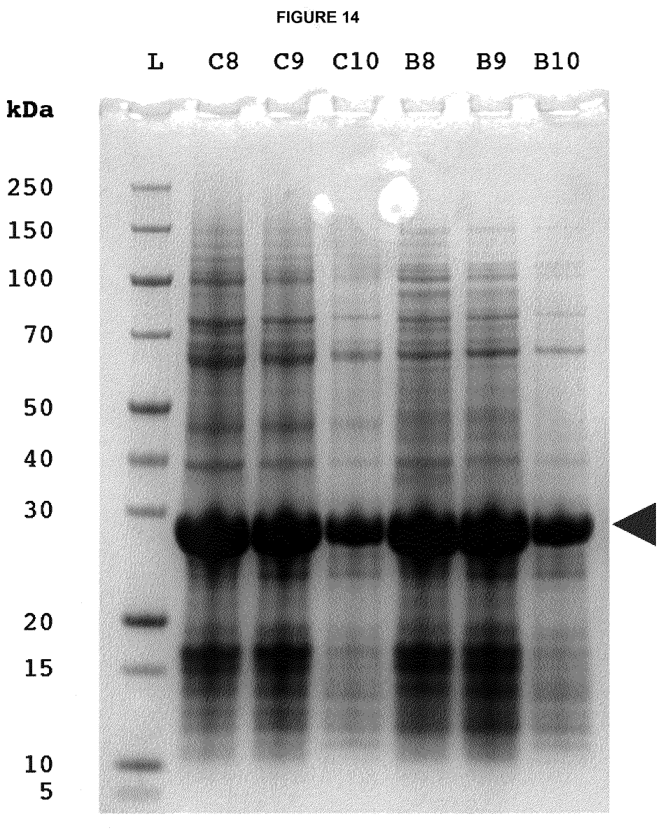

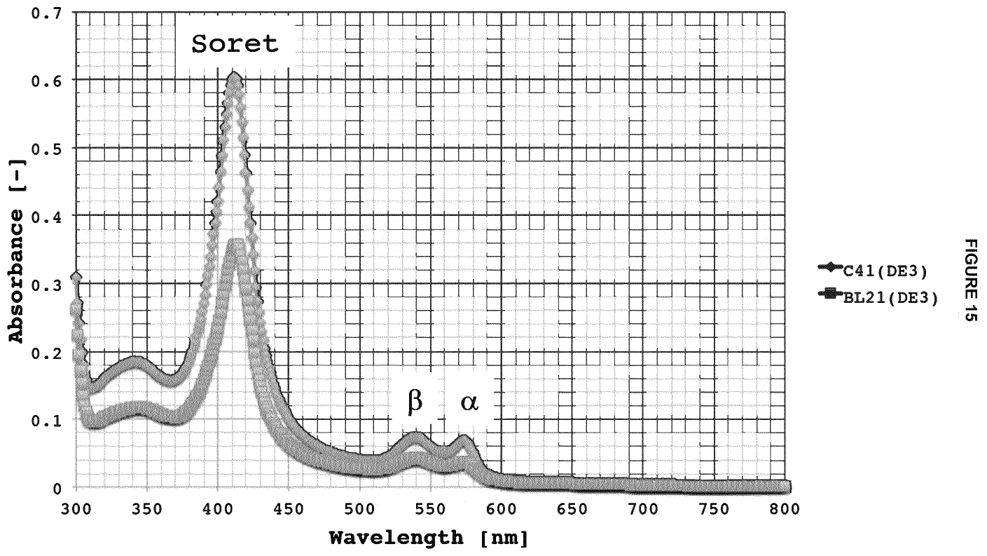

Protein fractions were analyzed on a NuPAGE™ 4-12%, Bis-Tris, 1 mm, 12-well mini protein gel (Thermo Fisher Scientific) to check for size, purity, and integrity. The gel was run using NuPAGE™ MES SDS running buffer (Thermo Fisher Scientific) at a constant voltage of 200 V for 40 min. For visualization, the gel was stained using InstantBlue™ (Expedeon) (FIGS. 13 and 14).

### Example 7: Protein Quantification

Purified HLTev-VsLegH and HLTev-LlLegH were diluted with water, and quantified using Pierce™ Coomassie Plus (Bradford) Assay Kit (Thermo Fisher Scientific). Bovine serum albumin (2.5 μg/mL to 25 μg/mL) was used as a protein calibration standard. Briefly, 100 μL of Bradford reagent was added to 100 μL of diluted protein sample in a 96-well microplate. After a 30-sec shaking, the plate was incubated at room temperature for 10 min. Absorbance was then measured at 595 nm using a Multiskan™ FC microplate photometer (Thermo Fisher Scientific). Protein yields were shown in Table 1 given below.

TABLE 1
Protein yield after purification using a HisTrap ™ HP
column (Cytiva)

| | | Protein per | Protein per |
|---|---|---|---|
| | | 50 mL of | L of |
| Protein | Expression host | culture [mg] | culture [mg] |
| HLTev-VsLegH | E. coli C41(DE3) | 26.5 | 530 |
| HLTev-VsLegH | E. coli BL21(DE3) | 25.1 | 502 |
| HLTev-LILegH | E. coli C41(DE3) | 21.0 | 421 |
| HLTev-LILegH | E. coli BL21(DE3) | 18.0 | 360 |

### Example 8: UV-Vis Measurement

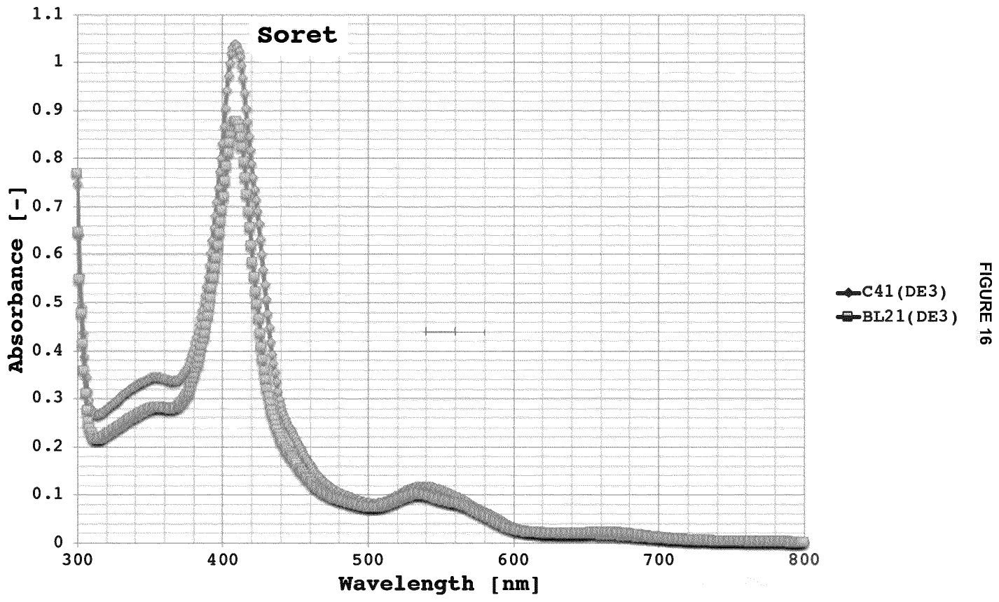

Protein samples were diluted with buffer B. Wavelength scan, from 800 nm to 300 nm, was conducted using UV-3100PC UV-Vis spectrophotometer (VWR). All protein samples displayed typical heme spectra (FIGS. 15 and 16).

### II. Yeast Production System

### Example 1: Molecular Cloning of Genes Encoding VsLegH, LlLegH and BtMyg

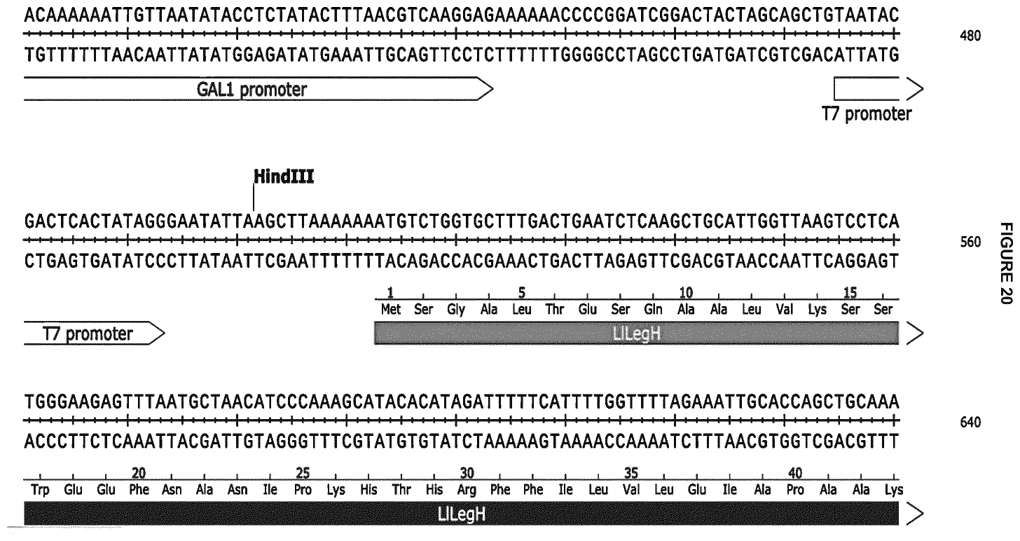

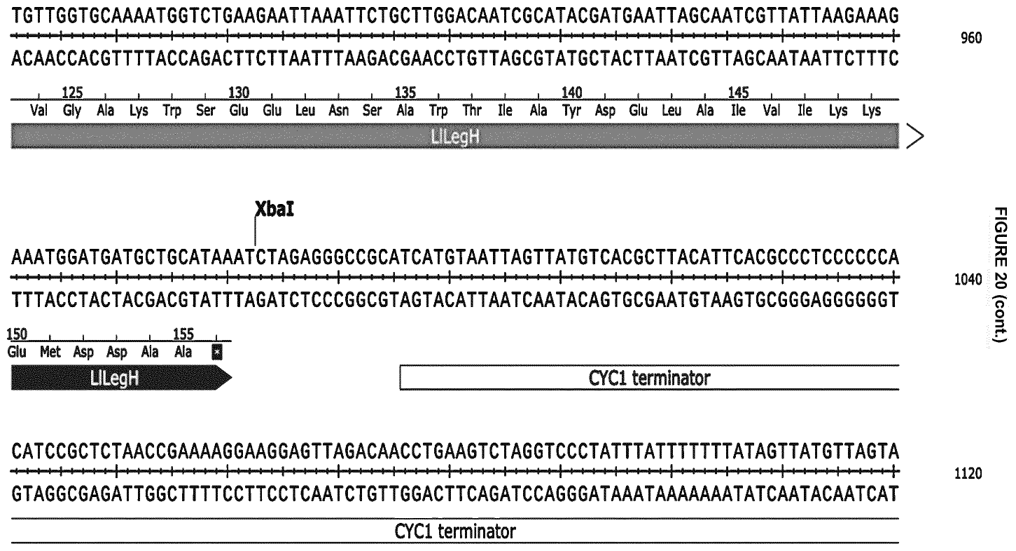

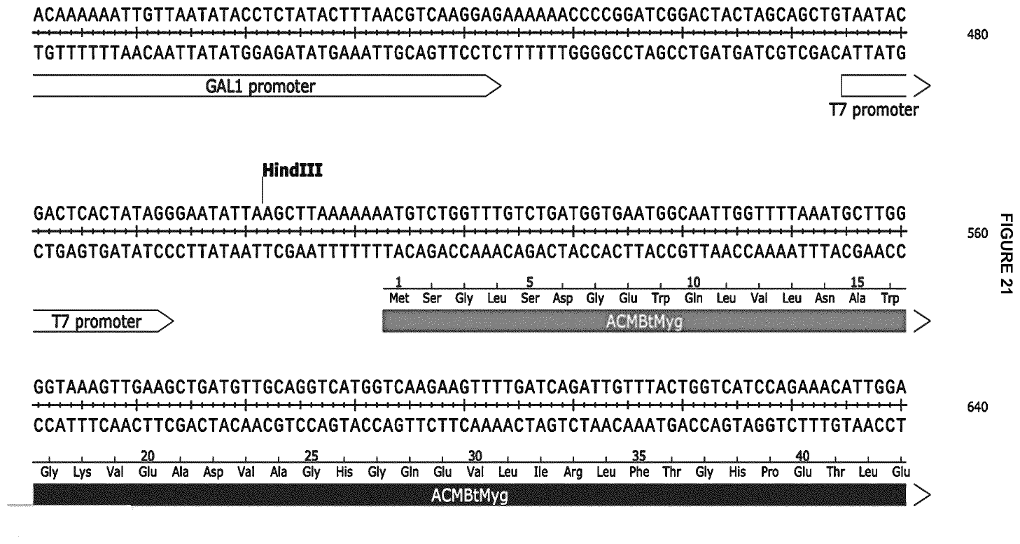

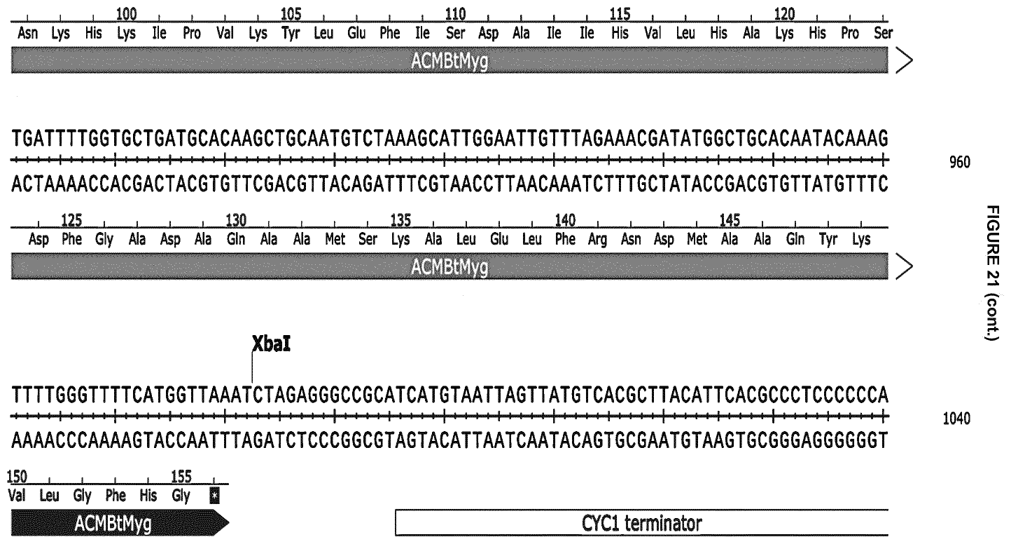

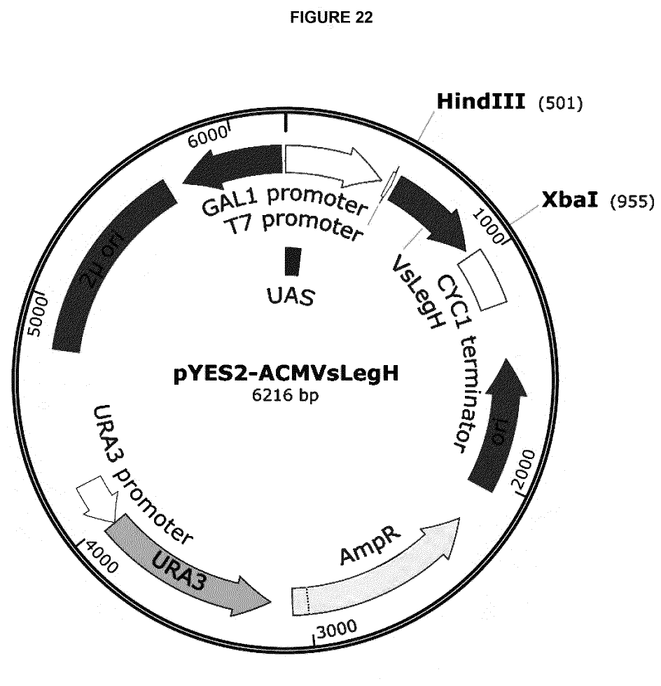

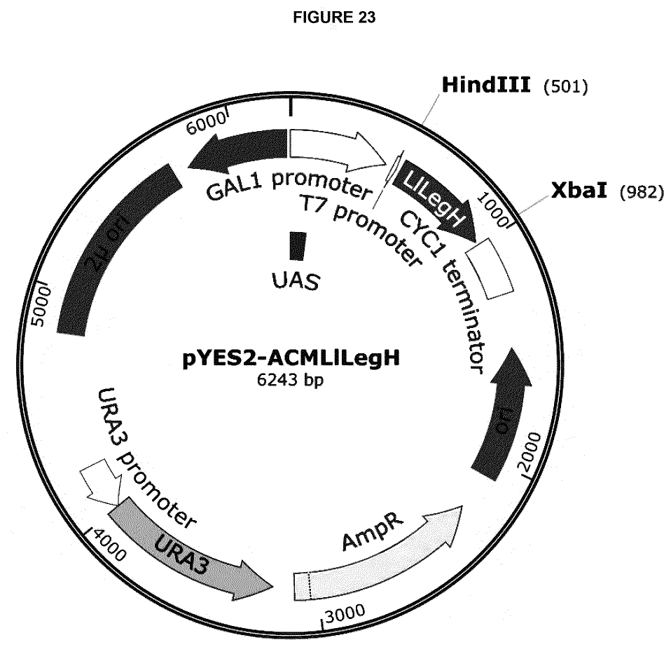

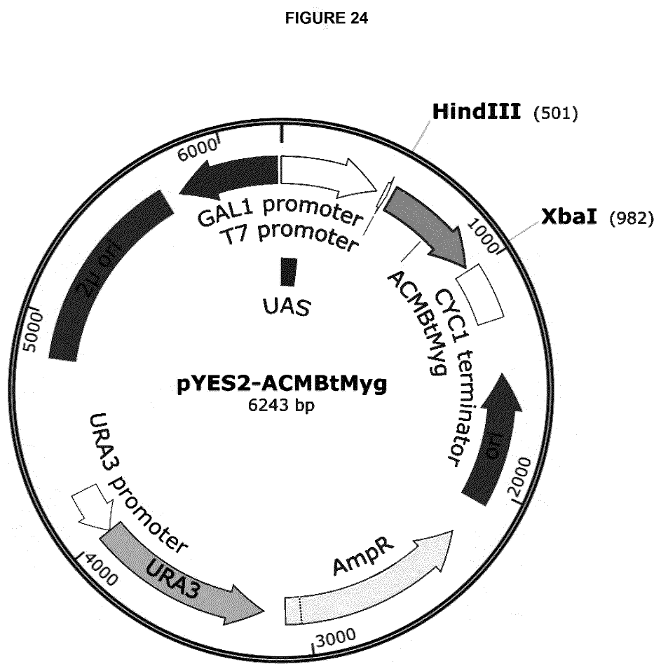

Genes encoding VsLegH, LlLegH, and BtMyg (SEQ ID NOs: 1, 3 and 5) were codon optimized for protein expression in Saccharomyces cerevisiae and cloned into pYES2 vector using HindIII and XbaI sites, as shown in FIGS. 19-21. The full plasmid maps of pYES2-ACMVsLegH, pYES2-ACMLlLegH and pYES2-ACMBtMyg were provided in FIGS. 22-24. GAL1 promoter was used for the protein expression. However, other yeast promoters, like GAL1, GAL10, GALL, GALS, CTR1, CTR3, CUP1, CYC1, MET25, promoter of glyceraldehyde 3-phosphate dehydrogenase (GPD), promoter of alcohol dehydrogenase 1 (ADH1), promoter of transcriptional elongation factor EF-1α (TEF1), promoter of transcriptional elongation factor EF-1α (TEF2), promoter of phosphoglycerate kinase (PGK1), promoter of triose phosphate isomerase (TP11), promoter of hexose transporter (HXT7), promoter of pyruvate kinase 1 (PYK1), promoter of triose phosphate dehydrogenase (TDH3) may also be used.

### Example 2: N-Terminal Modification of VsLeqH, LlLegH and BtMyg

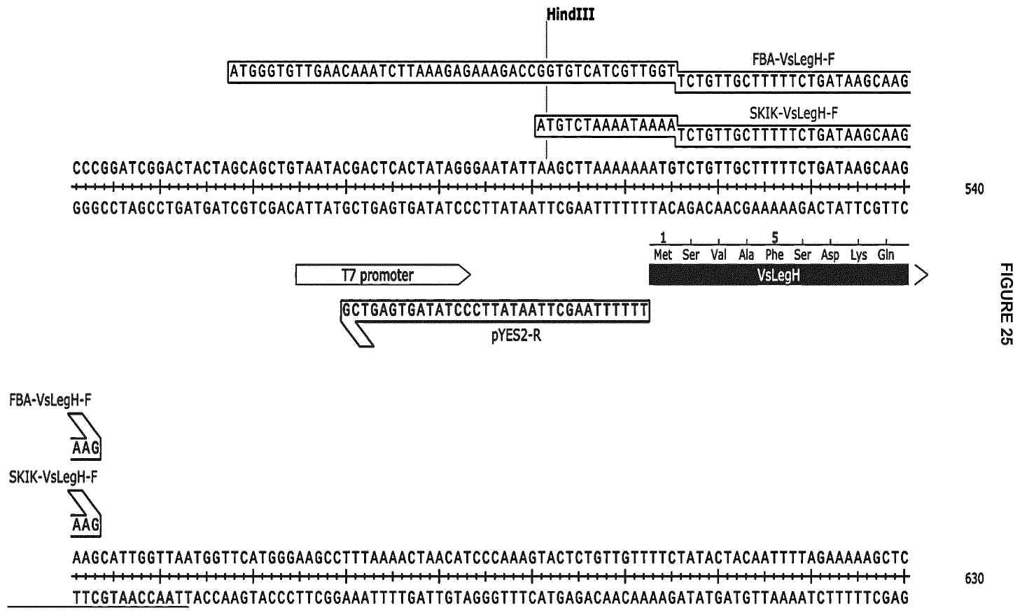

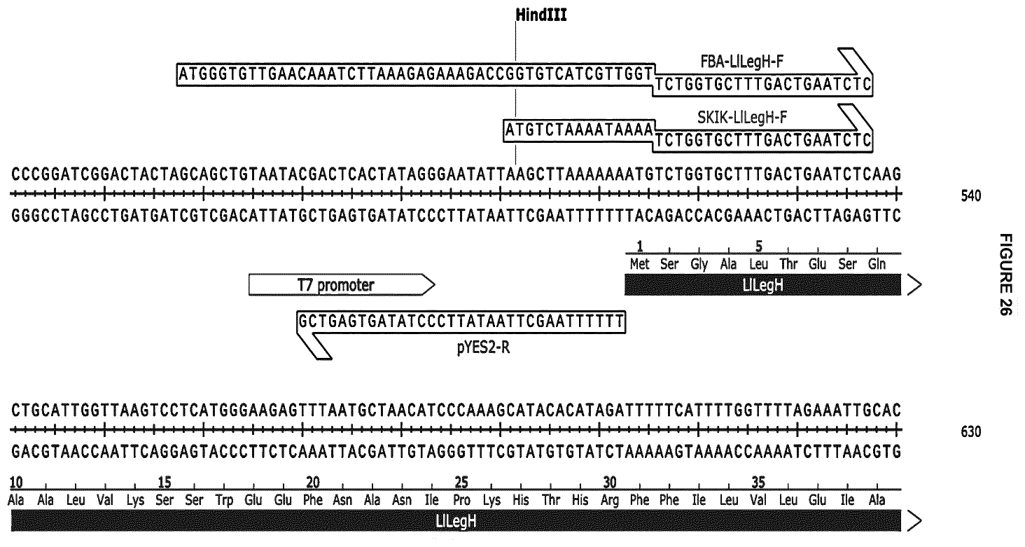

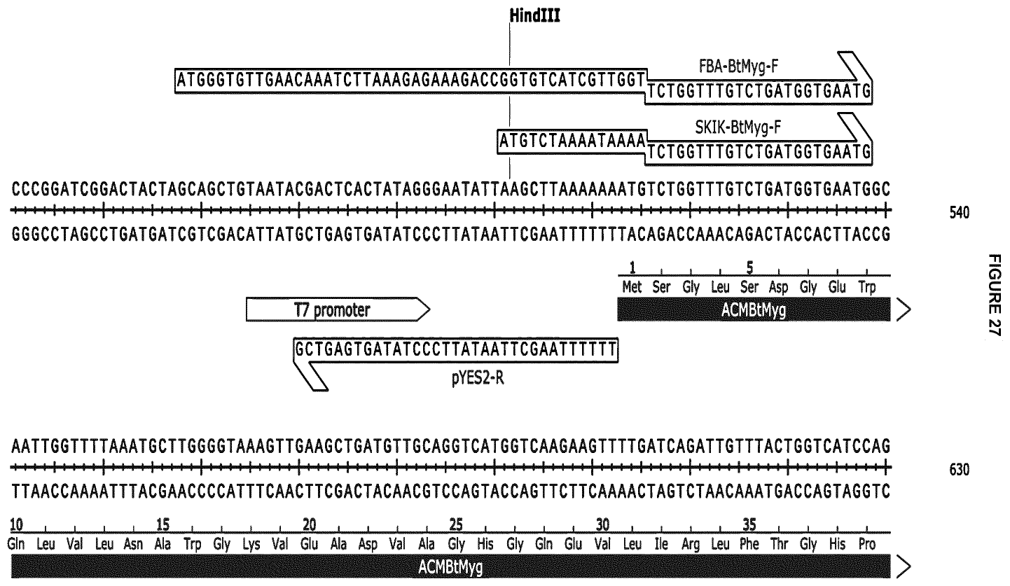

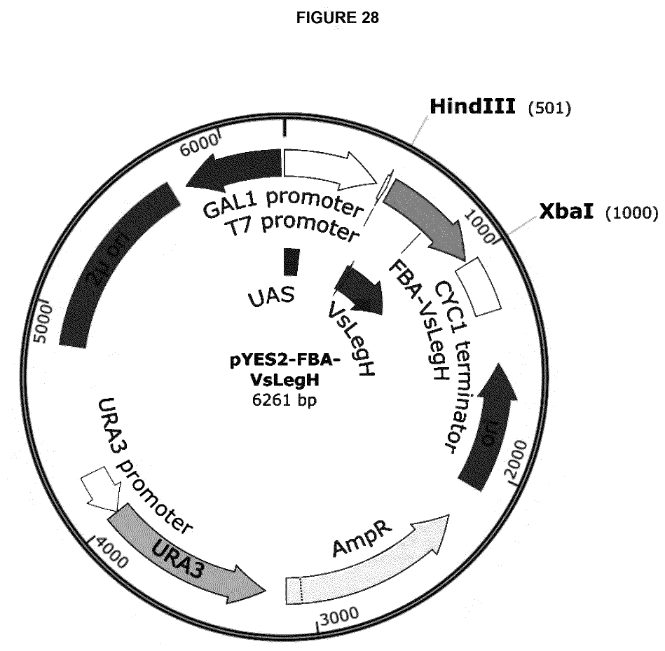

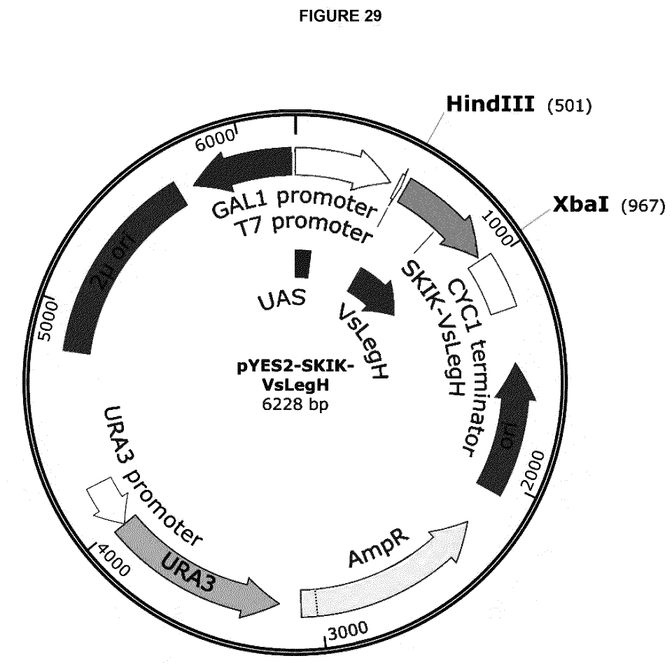

To improve the protein expression in S. cerevisiae, an FBA tag or an SKIK tag (protein sequences are given in SEQ ID NO: 11 and SEQ ID NO: 12) was introduced to the N-terminus of the protein, using the primers given in SEQ ID NOs: 13-19 (see also FIGS. 25-27) and a modified Q5 Site-Directed Mutagenesis protocol. The full plasmid maps of pYES-FBA-VsLegH and pYES-SKIK-VsLegH were provided in FIGS. 28-29.

### Example 3: Plasmid Transformation into S. cerevisiae INVSc1

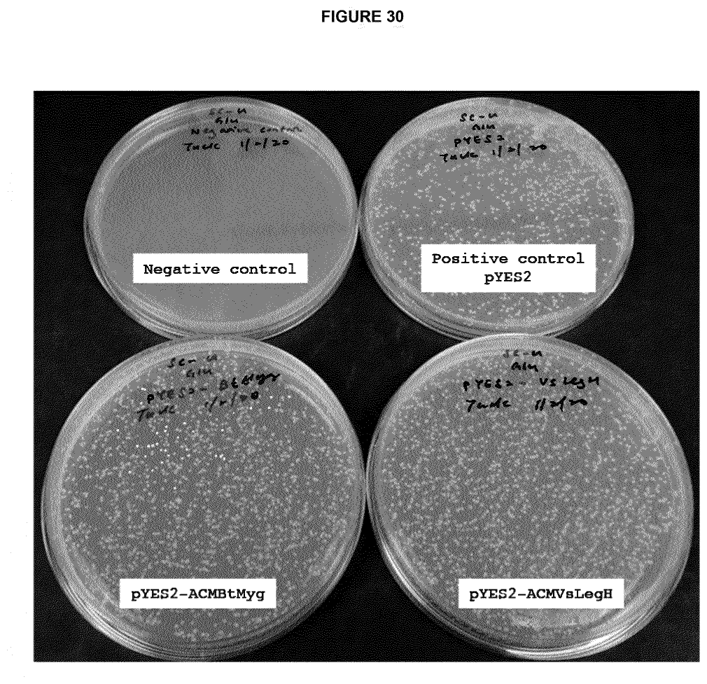

S. cerevisiae INVSc1 cells were streaked on YPD agar plate (medium composition was per L: 10 g yeast extract, 20 g peptone, 20 g D-glucose). A single colony was used to prepare an overnight culture in YPD medium. One pg plasmid DNA was used to transform S. cerevisiae INVSc1 cells using the Frozen-EZ Yeast Transformation II kit (Zymo Research). Transformed cells were plated on SC-U Glu agar plate (medium composition, per L, 6.9 g yeast nitrogen base without amino acid (Formedium), 0.77 g CSM, Single drop-out-Ura (Formedium), 20 g D-glucose, FIG. 30).

To improve the heme production in S. cerevisiae, and therefore the improved production of heme-incorporated globin (VsLegH, LlLegH or BtMyg), globin-expressing plasmid (e.g., pYES2-SKIK-VsLegH or pYES2-FBA-VsLegH; 0.5 μg) and heme-over-expressing plasmid (0.5 μg; H3 or H3H2H12) were co-transformed into S. cerevisiae INVSc1 cells, using the Frozen-EZ Yeast Transformation II kit (Zymo Research). Transformed cells were plated on SC-U-H Glu agar plate (medium composition was per L, 6.9 g yeast nitrogen base without amino acid (Formedium), 0.77 g CSM, Double drop-out-Ura-His (Formedium), 20 g D-glucose; FIG. 31). Plasmids H3 and H3H2H12 were kind gifts from Prof. Jens B. Nielsen (Chalmers University of Technology, Sweden).

### Example 4: Small-Scale Protein Expression

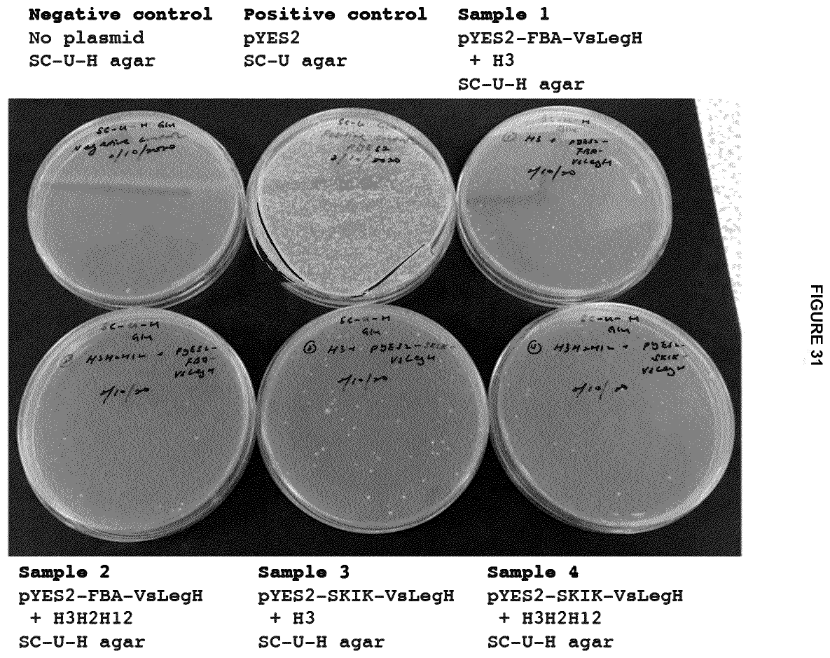

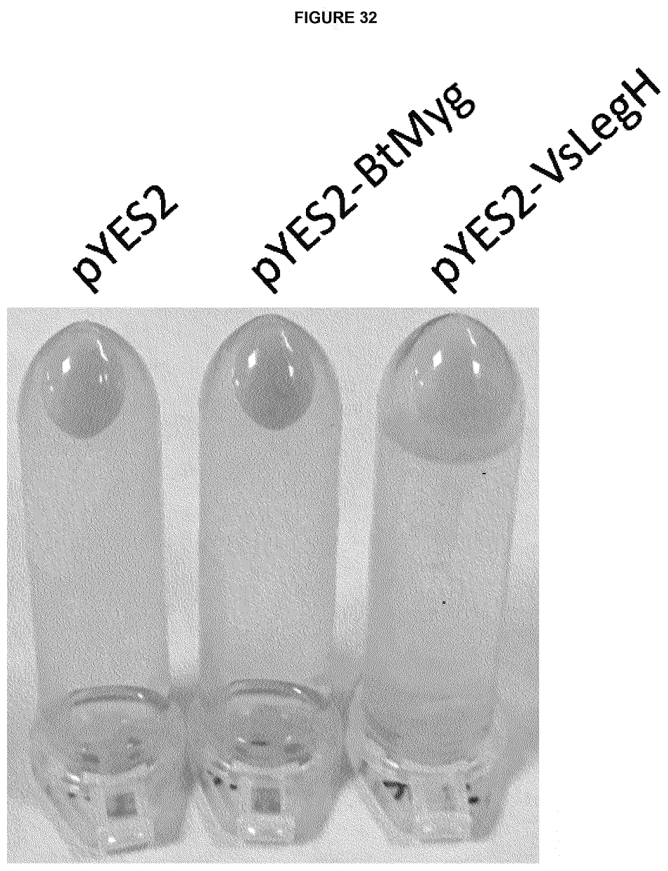

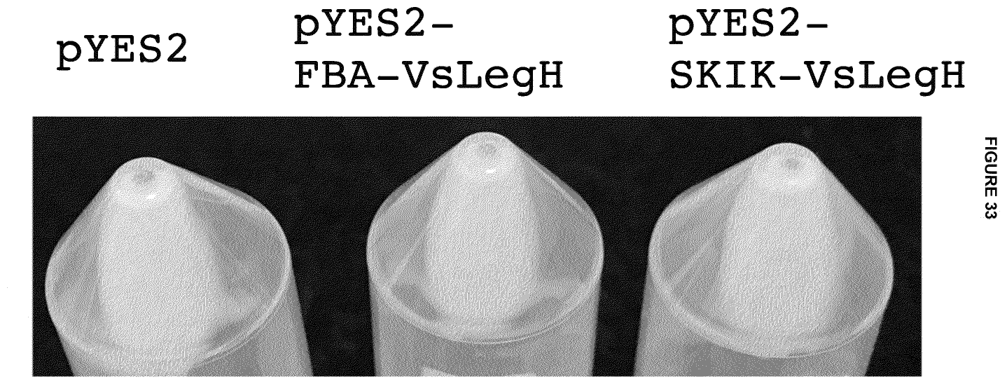

An overnight culture was prepared by inoculating a single colony of S. cerevisiae INVSc1 cells harbouring a globin-expressing pYES2 plasmid into 5 mL of SC-U Glu medium (medium composition was per L, 6.9 g yeast nitrogen base without amino acid (Formedium), 0.77 g CSM, Single drop-out-Ura (Formedium), 20 g D-glucose). The culture was incubated at 30° C. and 200 rpm. The OD₆₀₀ value of the overnight culture was measured to determine the amount of overnight culture required to obtain an OD₆₀₀ value of 0.4 in 50 mL of induction medium (SC-U Gal; medium composition was per L, 6.9 g yeast nitrogen base without amino acid (Formedium), 0.77 g CSM, Single drop-out-Ura (Formedium), 20 g galactose). The amount of overnight culture required was pelleted by centrifugation at 1500 g for 5 min at 4° C. The cell pellet was resuspended in 1-2 mL of induction medium (SC-U Gal) and inoculated into 50 ml of induction medium (SC-U Gal). The culture was incubated at 30° C. and 200 rpm. After 24 hrs, cells were harvested and stored at −80° C. (FIGS. 32-34).

For globin expression in S. cerevisiae INVSc1 cells harbouring a heme-overexpressing plasmid (H3 or H3H2H12), the medium for preparing overnight culture and the induction medium were changed to SC-U-H Glu and SC-U-H Gal, respectively (medium composition of SC-U-H Gal: per L, 6.9 g yeast nitrogen base without amino acid (Formedium), 0.77 g CSM, Double drop-out-Ura-His (Formedium), 20 g galactose).

### Example 5: Protein Extraction

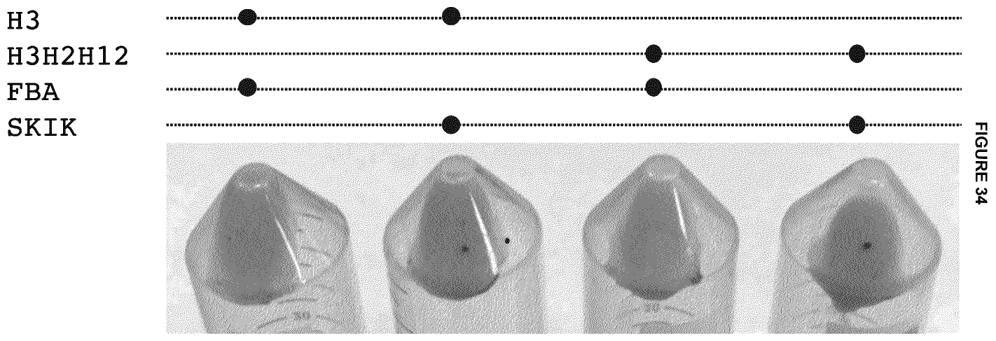

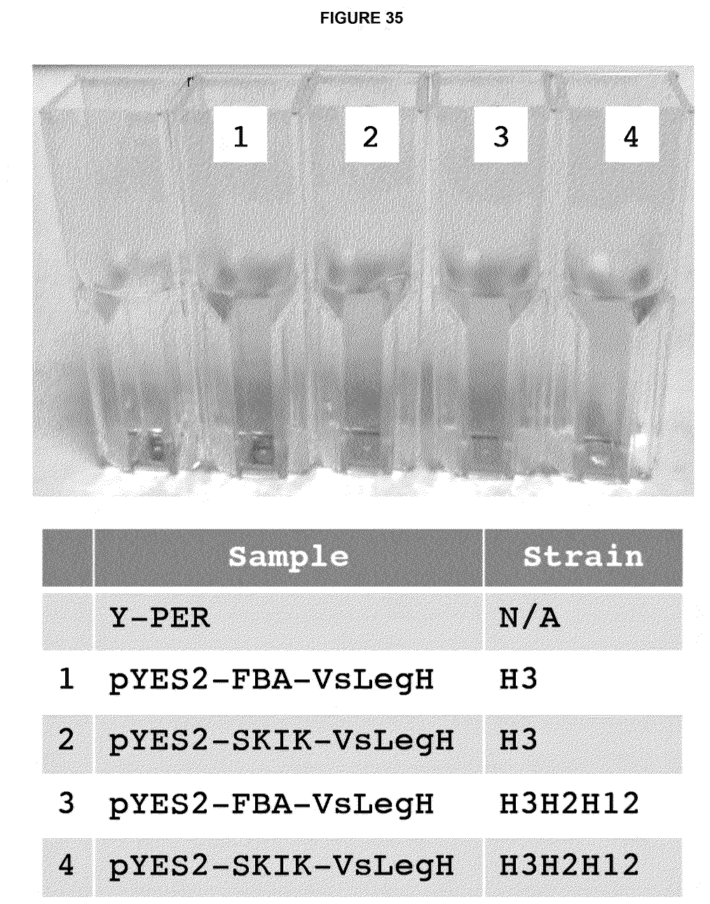

For protein extraction from S. cerevisiae INVSc1 cells, Y-PER™ Yeast Protein Extraction Reagent (Thermo Fisher Scientific) was used. To avoid proteolytic cleavage, a tablet of Pierce Protease Inhibitor Mini Tablet (A32955; Thermo Fisher Scientific) was added to 15 mL of Y-PER. Cell pellet from 50 mL of expression was resuspended in 3.5 mL of Y-PER supplemented with protease inhibitor. The mixture was agitated at room temperature for 20 min. Cell debris was pelleted by centrifugation at 14000 g for 10 min. The cell extract (FIG. 35) was subsequently used for SDS-PAGE and UV-Vis measurement.

### Example 6: SDS-PAGE

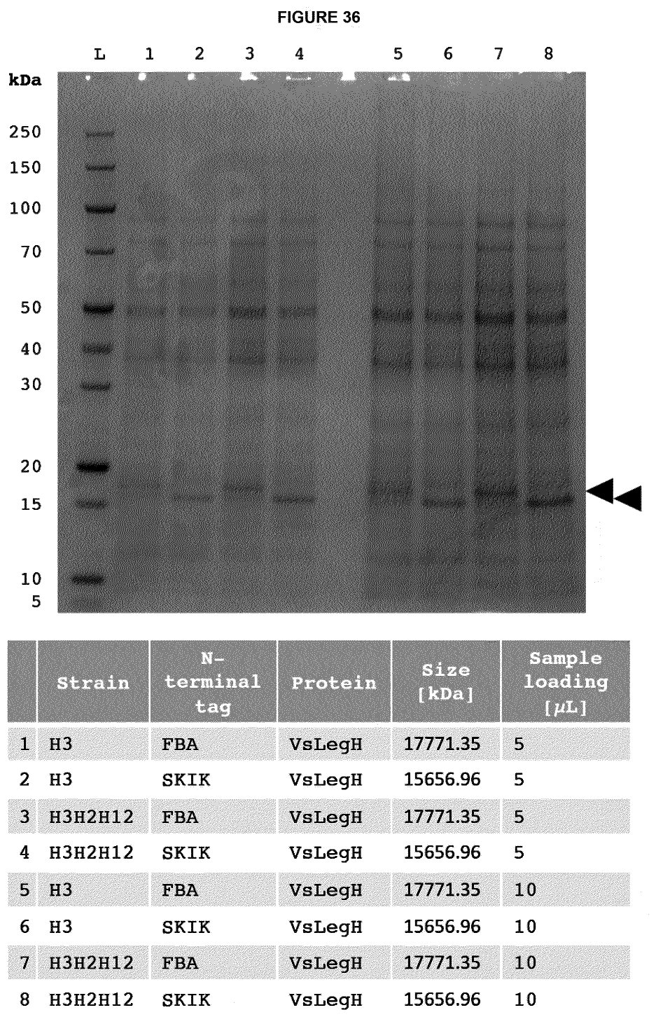

Protein fractions were analyzed on a NuPAGE™ 4-12%, Bis-Tris, 1 mm, 12-well mini protein gel (Thermo Fisher Scientific) to check for size and integrity. The gel was run using NuPAGE™ MES SDS running buffer (Thermo Fisher Scientific) at a constant voltage of 200 V for 40 min. For visualization, the gel was stained using InstantBlue™ (Expedeon) (FIG. 36).

### Example 7: UV-Vis Measurement

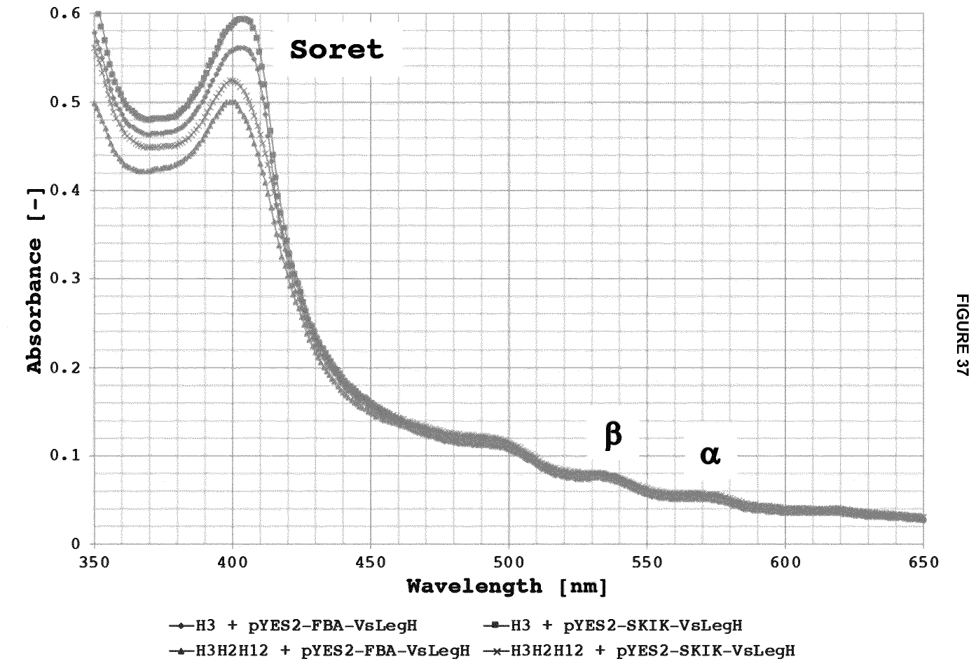

Wavelength scan, from 800 nm to 300 nm, was conducted using UV-3100PC UV-Vis spectrophotometer (VWR). All protein samples displayed typical heme spectra (FIG. 37).

### Example 8: Investigation of Further LegH Genes

In addition to the VsLegH (isolated originally from Vigna subterranea) and LlLegH (from Lupinus luteus), the inventors of the present invention tested additional LegH genes. The inventors of the present invention mainly aimed at identifying a LegH that is functionally expressed well in Escherichia coli, identifying a LegH that shows high heme incorporation, and therefore, more intense reddish colour, and identifying a LegH that is more stable, and therefore, provides easier bioprocess development for a large-scale LegH production.

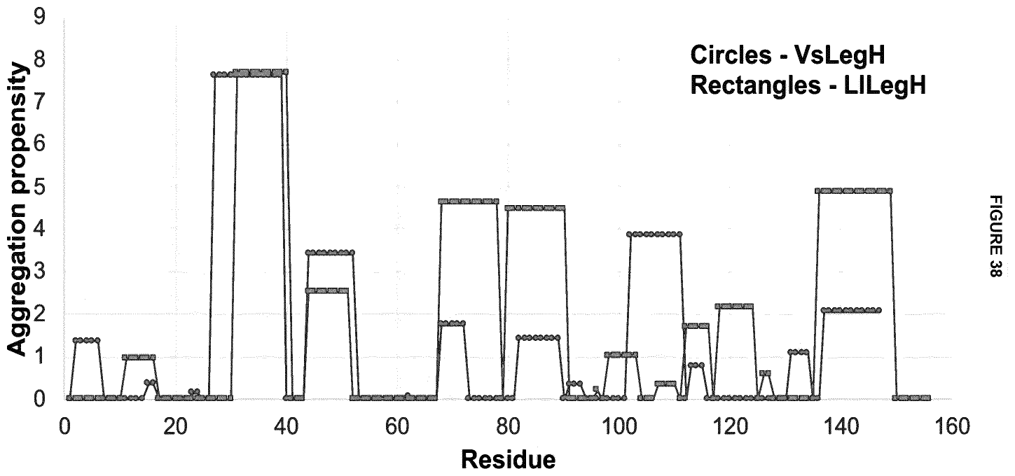

When the inventors processed VsLegH and LlLegH, they noticed that LlLegH differs from VsLegH in two aspects: 1) LlLegH has lower heme incorporation in comparison to VsLegH, although both genes are expressed well in Escherichia coli. 2) LlLegH is prone to protein aggregation. This observation is corroborated with the Aggrescan analysis given in FIG. 38.

To search for further LegH, the inventors conducted a protein BLAST using the protein sequence of LlLegH as query sequence. From the list of similar protein sequences (not shown herein), the inventors picked the following 4 sequences for further sequence analysis:
1) Leghemoglobin-2 [Lupinus angustifolius] (NCBI GenBank accession: XP_019460384.1) (denoted in the following as LaLegH2; SEQ ID NO: 24).
2) Leghemoglobin-1 [Lupinus angustifolius] (NCBI GenBank accession: XP_019433600.1) (denoted in the following as LaLegH1; SEQ ID NO: 23).
3) Hemoglobin I, leg [Lupinus luteus] (NCBI GenBank accession: 0607193A) (denoted as LlLegH1; SEQ ID NO: 25).
4) Hypothetical protein Lalb_Chr10g0098701 [Lupinus albus] (NCBI GenBank accession: KAE9605566.1) (denoted in the following as LalLegH; SEQ ID NO: 26).

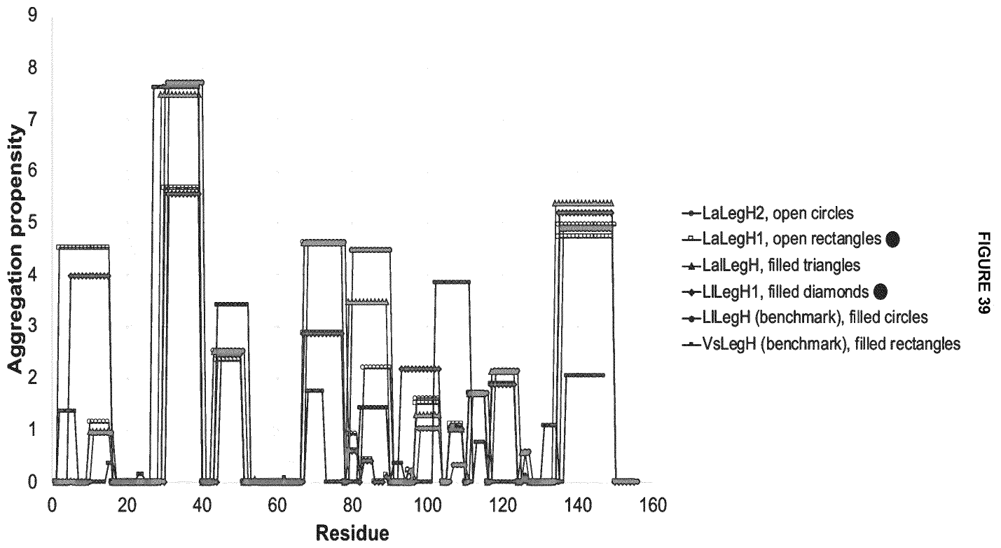

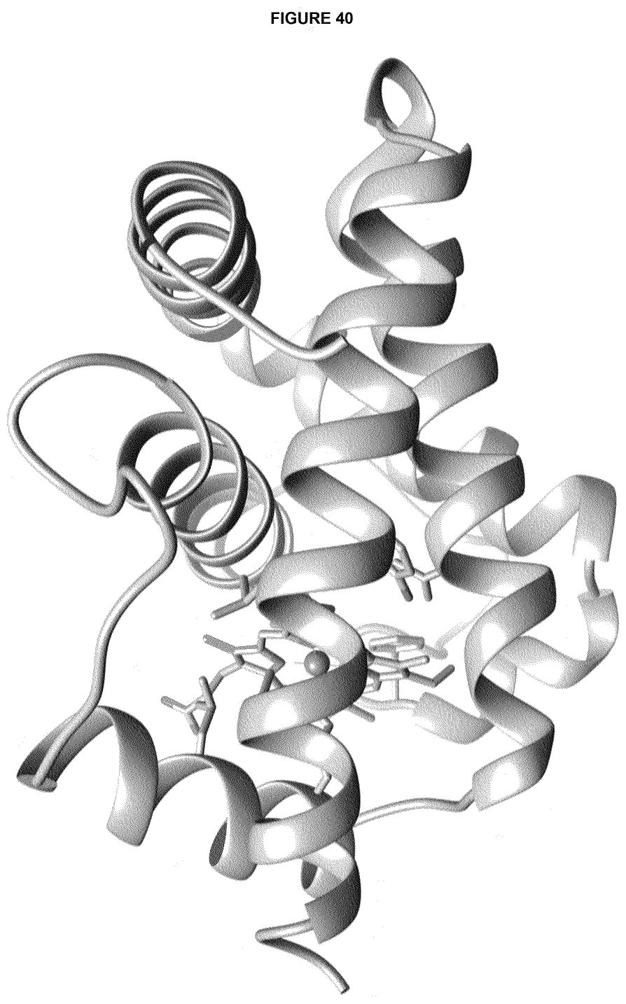

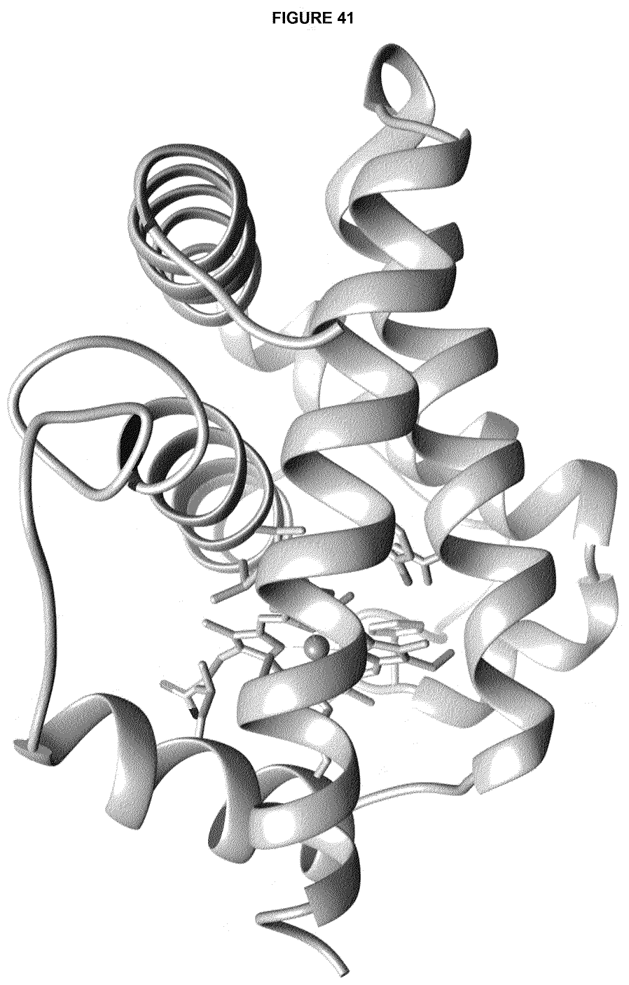

After Aggrescan analysis of the 4 sequences of LaLegH1 (SEQ ID NO: 23), LaLegH2 (SEQ ID NO: 24), LlLegH1 (SEQ ID NO: 25) and LalLegH (SEQ ID NO: 26) (see FIG. 39), the inventors decided to focus on LaLegH1 (SEQ ID NO: 23) and LlLegH1 (SEQ ID NO: 25), as they have lower aggregation propensity. Aggrescan is a web-based software for the prediction of aggregation-prone segments in protein sequences. Aggregation propensity is defined as the tendency of a protein sequence to aggregate. Based on protein models (see FIGS. 40 and 41), performed using SWISS-MODEL, LaLegH1 (SEQ ID NO: 23) and LlLegH1 (SEQ ID NO: 25) are hemoproteins, with predicted heme binding site.

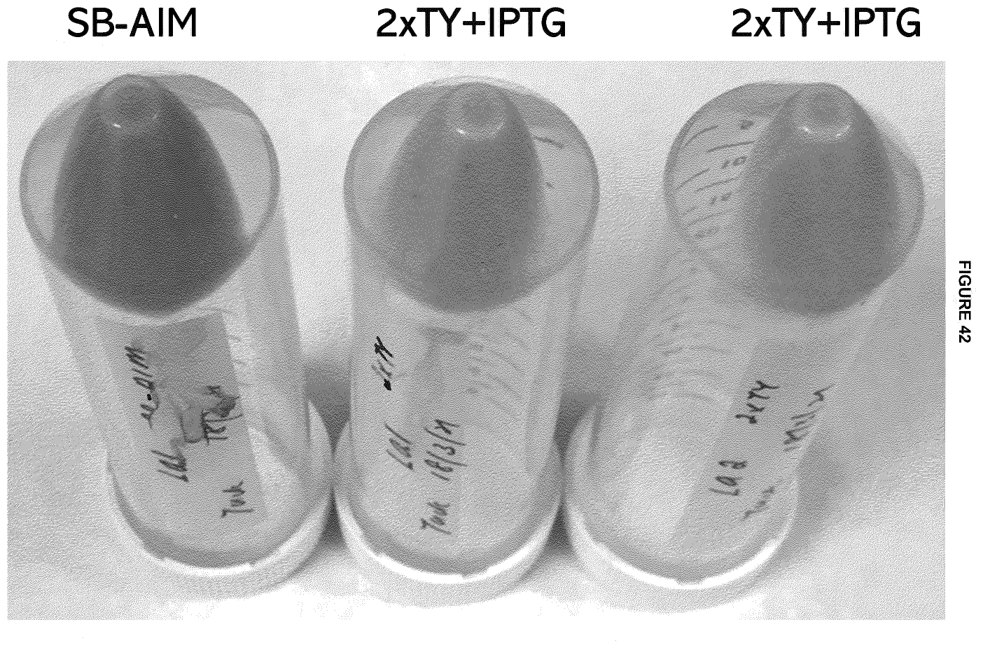

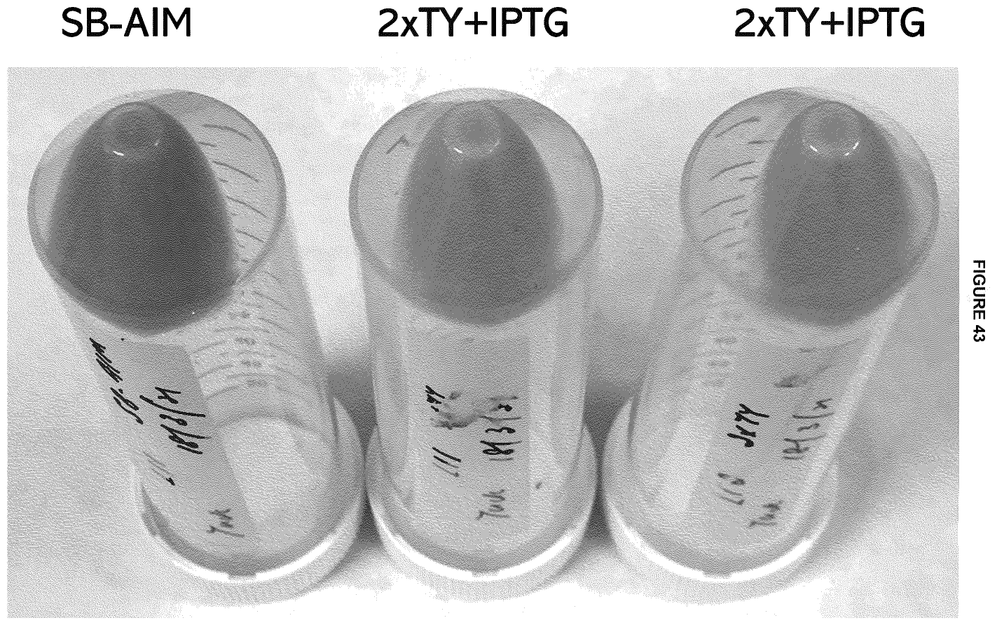

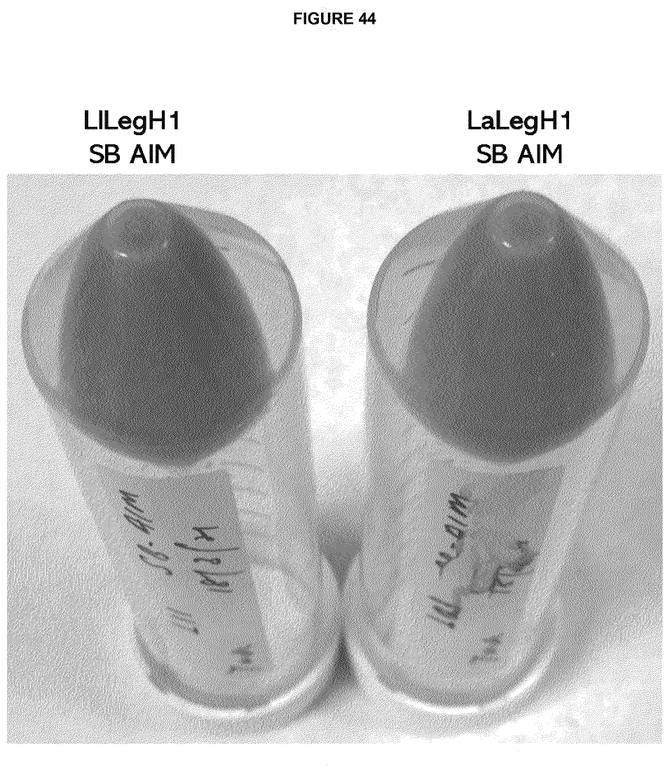

The genes encoding LaLegH1 and LlLegH1 (SEQ ID NO: 27 and 28) were codon optimised for E. coli expression and cloned into the pET24a-HLTev vector. It was confirmed that they are both hemoproteins, judging on the reddish colour the proteins gave (see FIGS. 42 and 43). Compared to LaLegH1, LlLegH1 either expressed better or had better heme incorporation. Its reddish colour is more intense compared to that of LaLegH1 (see FIG. 44).

### Example 9: Further Expression System for LegH

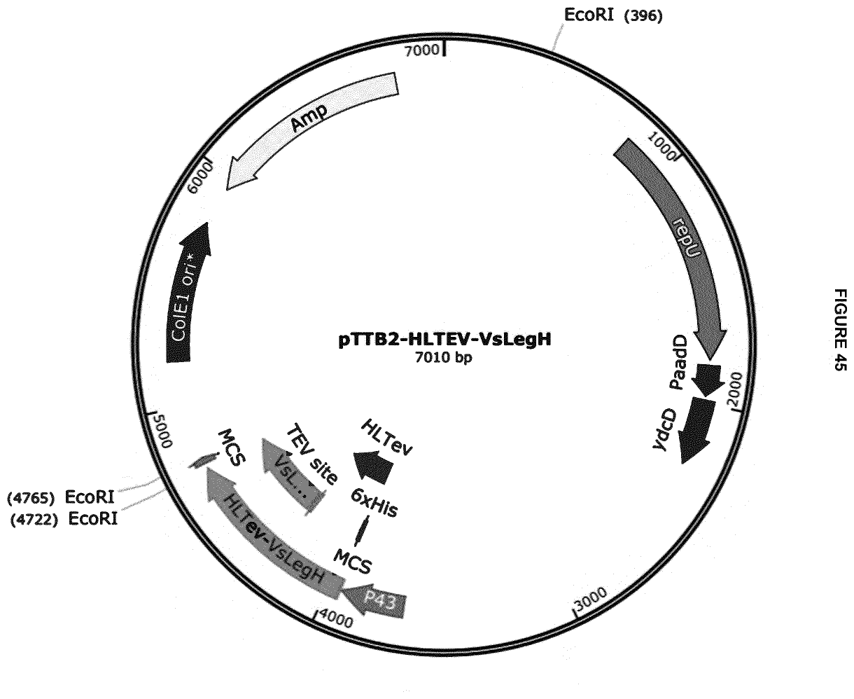

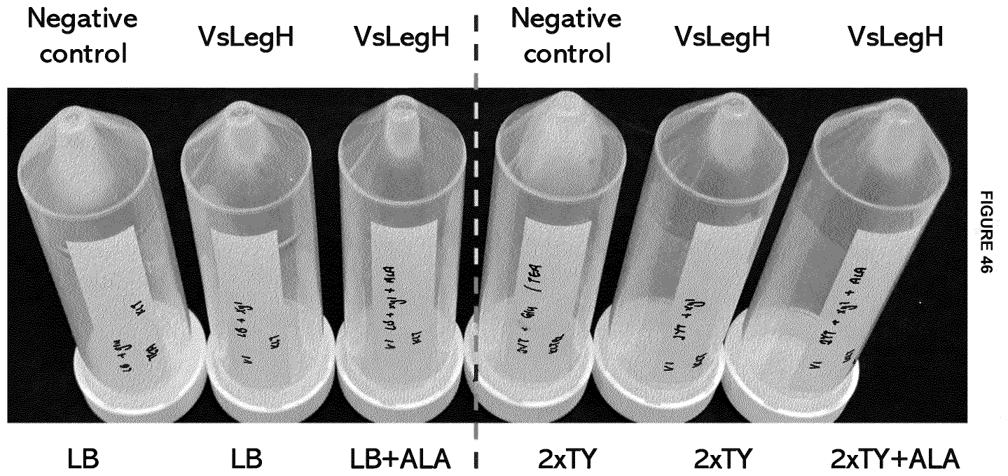

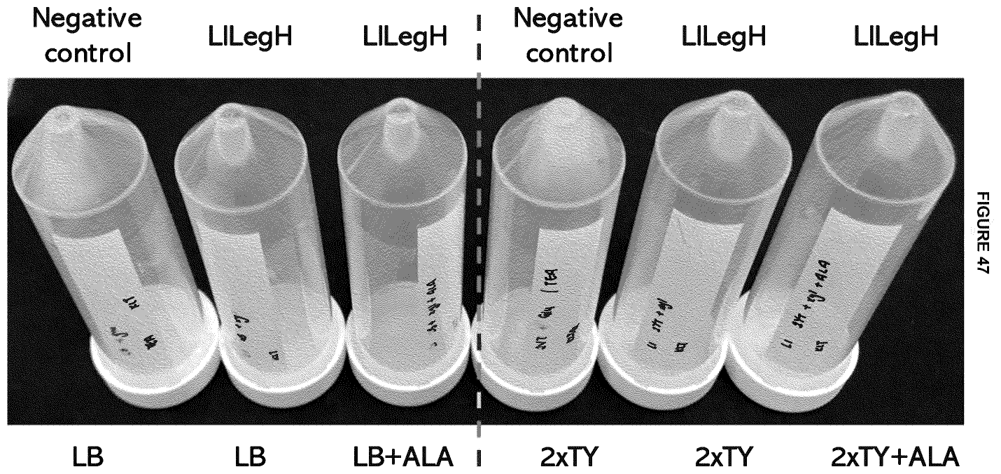

In addition to Escherichia coli and Saccharomyces cerevisiae, as described herein, the inventors have tested other bacterial expression system, specifically Bacillus subtilis TEA strain. The genes encoding HLTev-VsLegH and HLTev-LlLegH (SEQ ID NO: 29 and 30) were cloned into the pTTB2 vector (see FIG. 45). Expression in Escherichia coli was higher than expression in Bacillus subtilis TEA strain (see FIGS. 46 and 47).

### Example 10: Further Investigation of Vigna subterranea

In the genome of most species, the inventors noticed multiple homologs of leghemoglobin. For example, when looking at soybean (Glycine max), there are at least 4 homologs:

Leghemoglobin C1 (NCBI GenBank accession: NP_001345001.1; SEQ ID NO: 31); Leghemoglobin C2 (NCBI GenBank accession: NP_001235248.2; SEQ ID NO: 32); Leghemoglobin C3 (NCBI GenBank accession: NP_001235423.1; SEQ ID NO: 33); and Leghemoglobin A (NCBI GenBank accession: NP_001235928.1; SEQ ID NO: 34).

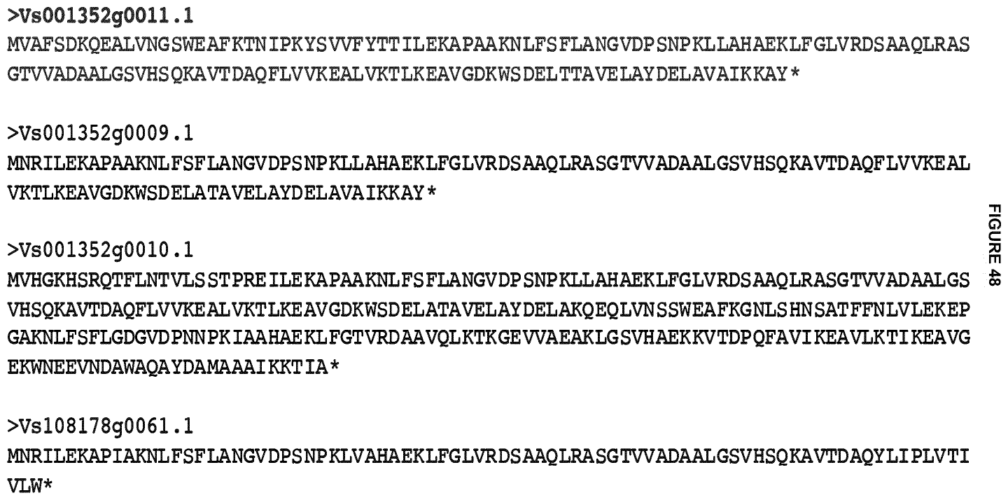

Due to that, the inventors of the present invention made further investigations of the Vigna subterranea genome. The inventors used the protein sequence of VsLegH to BLAST against the V. subterranea genome and identified 4 sequences (see FIG. 48):

Vs001352g0011.1 (VsLegH according to the present invention; SEQ ID NO: 2), Vs001352g0009.1 (denoted as VsLegH9; SEQ ID NO: 35), Vs001352g0010.1 (denoted as VsLegH10; SEQ ID NO: 36), and Vs108178g0061.1 (denoted as VsLegH61; SEQ ID NO: 37).

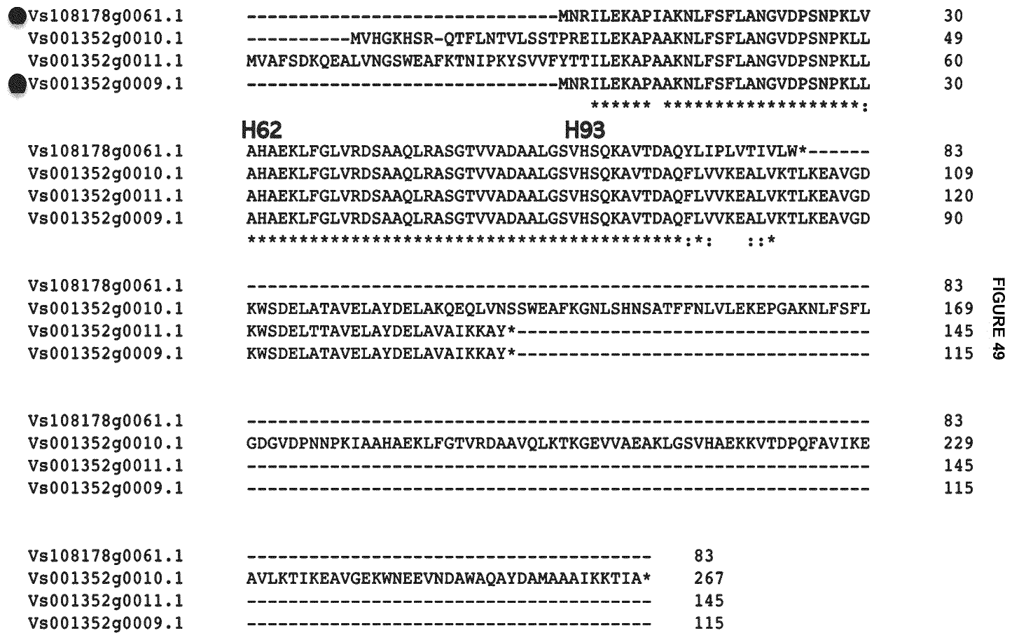

Sequence alignment: The sequence alignment of the 4 sequences (see FIG. 49) showed that Vs001352g0009.1 (SEQ ID NO: 35) and Vs108178g0061.1 (SEQ ID NO: 37) are the ‘truncated’ versions of Vs001352g0011.1 (VsLegH according to the present invention) (SEQ ID NO: 2). Vs001352g0010.1 (SEQ ID NO: 36), on the other hand, can be seen as the ‘extended’ version of Vs001352g0011.1 (SEQ ID NO: 2). Interestingly, H62 and H93, which are responsible for heme binding, are conserved among all 4 sequences. These are likely homologs of leghemoglobin.

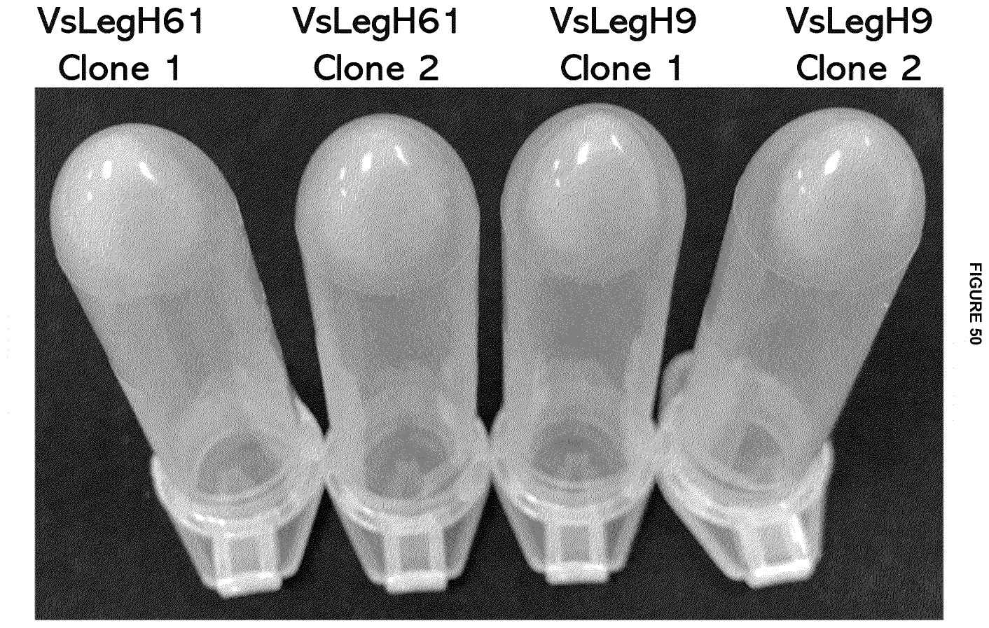

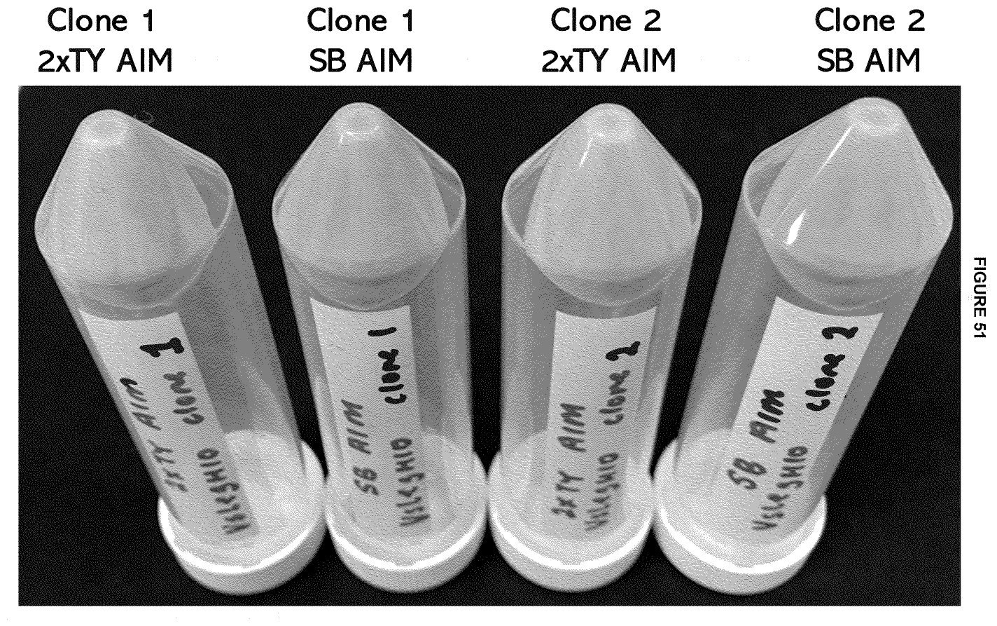

Protein expression: Protein expression in Escherichia coli showed that VsLegH9 (Vs001352g0009.1; SEQ ID NO: 35), VsLegH61 (Vs108178g0061.1; SEQ ID NO: 37) and VsLegH10 (Vs001352g0010.1; SEQ ID NO: 36) are hemoproteins (see FIGS. 50 and 51). VsLegH (Vs001352g0011.1; SEQ ID NO: 2) provided the highest expression/heme incorporation of said examples.

### Example 11: Bacterial Hemoglobin

Plant-based hemoglobins (such as VsLegH and LlLegH as described herein) can be recombinantly expressed according to the present invention in bacterial hosts, for instance, in Escherichia coli. However, the inventors of the present invention have also found that bacterial hemoglobins can be used in the present invention and can serve as meat surrogate. Preferred is the bacterial hemoglobin from Vitreoscilla species (e.g., Vitreoscilla stercoraria, Vitreoscilla sp. HG1, Vitreoscilla sp strain C1). The protein sequence of bacterial hemoglobin from Vitreoscilla stercoraria is given herein as SEQ ID NO: 38.

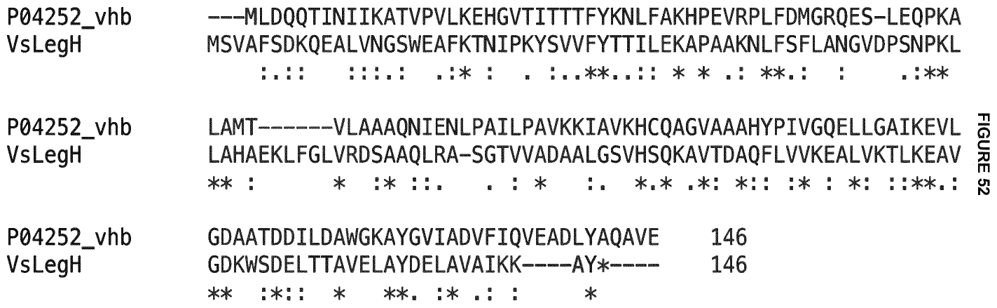

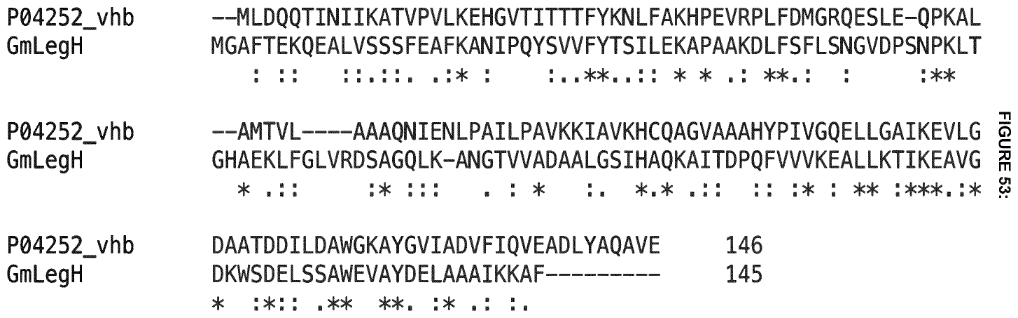

Despite having similar protein length, bacterial hemoglobin from Vitreoscilla stercoraria shows very low sequence identity with leghemolobin from Vigna subterranea (bambara groundnut, 25.44% identity, see FIG. 52) or from Glycine max (soybean, 34.78% identity, see FIG. 53), based on sequence alignment performed with Clustal Omega.

### REFERENCES

Martinez J. L., Liu L., Petranovic, D., Nielsen, J., Engineering the Oxygen Sensing regulation results in an Enhanced Recombinant Human Hemoglobin Production by Saccharomyces cerevisiae, Biotechnology and Bioengineering, Vol. 112, No. 1, 2015, pages 181-188.

1. Method of producing globin polypeptide recombinantly, comprising:
transforming a host cell with an expression vector comprising
one or more nucleic acid sequence(s) encoding said globin polypeptide, wherein said globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
wherein the host cell is selected from yeast or bacteria;
culturing said host cell under conditions effective for the expression of said one or more nucleic acid sequence(s);
expression of said one or more nucleic acid sequence(s), and
recovering said globin polypeptide from the obtained culture.

2. The method of claim 1, wherein the one or more nucleic acid sequence(s) encode(s) a leghemoglobin from a Vigna plant, preferably a Vigna plant selected from the group consisting of Vigna ambacensis, Vigna angivensis, Vigna filicaulis, Vigna friesiorum, Vigna gazensis, Vigna hosei, Vigna luteola, Vigna membranacea, Vigna monantha, Vigna racemosa, Vigna subterranea, and Vigna unguiculata, more preferably Vigna subterranea.

3. The method of claim 1, wherein the one or more nucleic acid sequence(s) encode(s) a leghemoglobin from a lupin, preferably a lupin selected from the group consisting of Lupinus albus, Lupinus angustifolius, Lupinus micranthus, Lupinus luteus, Lupinus hispanicus, Lupinus cosentinii, Lupinus digitatus, Lupinus princei, Lupinus pilosus, Lupinus palaestinus, Lupinus atlanticus, Lupinus mutabilis, Lupinus texensis, and Lupinus nootkatensis, more preferably Lupinus luteus.

4. The method of claim 1, wherein the one or more nucleic acid sequence(s) encode(s) a myoglobin from bovine, preferably a myoglobin from Bos taurus, Bos primigenius, Bos javanicus, Bos gaurus, Bos frontalis, Bos grunniens, Bos mutus, and Bos sauveli, more preferably Bos taurus.

5. The method of claim 1, wherein the one or more nucleic acid sequence(s) encode(s) a bacterial hemoglobin having at least 70% sequence identity with the bacterial hemoglobin of Vitreoscilla stercoraria, preferably wherein the one or more nucleic acid sequence(s) encode(s) the bacterial hemoglobin of Vitreoscilla stercoraria, of Vitreoscilla sp. HG1, or of the Vitreoscilla sp strain C1.

6. The method of claim 1, wherein the host cell is a bacterial host cell, preferably a bacterial host cell selected from the group consisting of Escherichia coli, Bacillus subtilis and Lactococcus lactis.

7. The method of claim 1, wherein the host cell is a yeast host cell, preferably a yeast host cell of the genus Saccharomyces, Pichia, Candida, Torulopsis or Hansenula, more preferably of Saccharomyces cerevisiae.

8. The method of claim 1, wherein said one or more nucleic acid sequence(s) is/are under regulation of a promoter or tandem promoter functional in bacteria or yeast, preferably wherein said promoter is a bacterial promoter, more preferably wherein the bacterial promoter is selected from the group consisting of the araBAD promoter, lac promoter, lacUV5 promoter, phoA promoter, pL promoter, pR promoter, rhaBAD promoter, Sp6 promoter, T3 promoter, T5 promoter, T7 promoter, T7lac promoter, tac promoter, tet promoter, trc promoter and the trp promoter, even more preferably the T7lac promoter; or, preferably wherein said promoter is a yeast promoter, more preferably wherein the yeast promoter is selected from the group consisting of the GAL1 promoter, GAL10 promoter, GALL promoter, GALS promoter, CTR1 promoter, CTR3 promoter, CUP1 promoter, CYC1 promoter, MET25 promoter, the promoter of glyceraldehyde 3-phosphate dehydrogenase (GPD), the promoter of alcohol dehydrogenase 1 (ADH1), the promoter of transcriptional elongation factor EF-1α (TEF1), the promoter of transcriptional elongation factor EF-1α (TEF2), the promoter of phosphoglycerate kinase (PGK1), the promoter of triose phosphate isomerase (TPI1), the promoter of hexose transporter (HXT7), the promoter of pyruvate kinase 1 (PYK1), and the promoter of triose phosphate dehydrogenase (TDH3), even more preferably the GAL1 promoter.

9. The method of claim 1, wherein the one or more nucleic acid sequence(s) encoding the globin polypeptide(s) comprise(s) or consist(s) of a sequence selected from the group consisting of SEQ ID NO: 1, SEQ ID NO: 3, SEQ ID NO: 5, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and fragments thereof.

10. Method of producing globin polypeptide with a cell-free translation system, comprising:
Providing one or more nucleic acid sequence(s) encoding the globin polypeptide, wherein the globin polypeptide is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof,
translating the one or more nucleic acid sequence(s) with the cell-free translation system, and
recovering the globin polypeptide from the cell-free translation system.

11. The method of claim 10, wherein the cell-free translation system is a bacterial cell-free system or a yeast cell-free system.

12. A food product, preferably a meat substitute food product, comprising:
one or more globin protein(s), wherein said one or more globin protein(s) is/are selected from the group consisting of a leghemoglobin from a Vigna plant, a leghemoglobin from a lupin, a myoglobin from bovine, a bacterial hemoglobin having at least 70% sequence identity with the bacterial hemoglobin of Vitreoscilla stercoraria, and any combination thereof;
one or more fibres, preferably one or more fibres from a plant, more preferably one or more fibres from a legume or a grain,
one or more carbohydrates, preferably one or more carbohydrates from a plant, more preferably one or more carbohydrates from a legume or a grain,
one or more fats, preferably one or more fats from non-animals,
one or more micronutrients,
one or more other proteins than the one or more globin protein(s), preferably one or more protein(s) from a plant, more preferably one or more protein(s) from a legume or a grain,
one or more flavors, yeast extracts, hydrolized vegetable proteins, herbs and/or seasoning, preferably plant flavors and/or plant seasoning,
optionally one or more egg albumin,
optionally one or more hydrocolloid,
optionally one or more mycoprotein, and
optionally one or more cell based protein.

13. The food product of claim 12, wherein the one or more globin protein comprise(s) or consist(s) of a sequence set forth in SEQ ID NO: 2, SEQ ID NO: 4, SEQ ID NO: 23, SEQ ID NO: 25, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or fragments thereof.

14. (canceled)

15. (canceled)

16. (canceled)
